SEQUENCE LISTING (82 sequences)

SEQ ID NO: 1 - DNA, 21 bp, Artificial Sequence
  Description of Artificial Sequence Combined DNA/RNA Molecule, Synthetic Oligonucleotide
cuuacgcugaguacuucgatt

SEQ ID NO: 2 - DNA, 21 bp, Artificial Sequence
  Description of Artificial Sequence Combined DNA/RNA Molecule, Synthetic Oligonucleotide
ucgaaguacucagcguaagtt

SEQ ID NO: 3 - DNA, 21 bp, Artificial Sequence
  Description of Artificial Sequence Combined DNA/RNA Molecule, Synthetic Oligonucleotide
cuuacgcugaguacuucgatt

SEQ ID NO: 4 - DNA, 21 bp, Artificial Sequence
  Description of Artificial Sequence Combined DNA/RNA Molecule, Synthetic Oligonucleotide
ucgaaguacucagcguaagtt

SEQ ID NO: 5 - DNA, 21 bp, Artificial Sequence
  Description of Artificial Sequence Combined DNA/RNA Molecule, Synthetic Oligonucleotide
cuuacgcugaguacuucgatt

SEQ ID NO: 6 - DNA, 21 bp, Artificial Sequence
  Description of Artificial Sequence Combined DNA/RNA Molecule, Synthetic Oligonucleotide
ucgaaguacucagcguaagtt

SEQ ID NO: 7 - DNA, 21 bp, Artificial Sequence
  Description of Artificial Sequence Combined DNA/RNA Molecule, Synthetic Oligonucleotide
cuuacgcugaguacuucgatt

SEQ ID NO: 8 - DNA, 21 bp, Artificial Sequence
  Description of Artificial Sequence Combined DNA/RNA Molecule, Synthetic Oligonucleotide
ucgaaguacucagcguaagtt

SEQ ID NO: 9 - DNA, 21 bp, Artificial Sequence
  Description of Artificial Sequence Combined DNA/RNA Molecule, Synthetic Oligonucleotide
cuuacgcugaguacuucgatt

SEQ ID NO: 10 - DNA, 21 bp, Artificial Sequence
  Description of Artificial Sequence Combined DNA/RNA Molecule, Synthetic Oligonucleotide
ucgaaguacucagcguaagtt

SEQ ID NO: 11 - DNA, 21 bp, Artificial Sequence
  Description of Artificial Sequence Combined DNA/RNA Molecule, Synthetic Oligonucleotide
cuuacgcugaguacuucgatt

SEQ ID NO: 12 - DNA, 21 bp, Artificial Sequence
  Description of Artificial Sequence Combined DNA/RNA Molecule, Synthetic Oligonucleotide
ucgaaguacucagcguaagtt

SEQ ID NO: 13 - DNA, 21 bp, Artificial Sequence
  Description of Artificial Sequence Combined DNA/RNA Molecule, Synthetic Oligonucleotide
cuuacgcugaguacuucgatt

SEQ ID NO: 14 - DNA, 21 bp, Artificial Sequence
  Description of Artificial Sequence Combined DNA/RNA Molecule, Synthetic Oligonucleotide
ucgaaguacucagcguaagtt

SEQ ID NO: 15 - DNA, 21 bp, Artificial Sequence
  Description of Artificial Sequence Combined DNA/RNA Molecule, Synthetic Oligonucleotide
cuuacgcugaguacuucgatt

SEQ ID NO: 16 - DNA, 21 bp, Artificial Sequence
  Description of Artificial Sequence Combined DNA/RNA Molecule, Synthetic Oligonucleotide
ucgaaguacucagcguaagtt

SEQ ID NO: 17 - DNA, 21 bp, Artificial Sequence
  Description of Artificial Sequence Combined DNA/RNA Molecule, Synthetic Oligonucleotide
cuuacgcugaguacuucgatt

SEQ ID NO: 18 - DNA, 21 bp, Artificial Sequence
  Description of Artificial Sequence Combined DNA/RNA Molecule, Synthetic Oligonucleotide
ucgaaguacucagcguaagtt

SEQ ID NO: 19 - RNA, 21 nt, Artificial Sequence
  Description of Artificial Sequence siRNA, Synthetic Oligonucleotide
cuuacgcugaguacuucgauu

SEQ ID NO: 20 - RNA, 21 nt, Artificial Sequence
  Description of Artificial Sequence siRNA, Synthetic Oligonucleotide
ucgaaguacucagcguaaguu

SEQ ID NO: 21 - RNA, 21 nt, Artificial Sequence
  Description of Artificial Sequence siRNA, Synthetic Oligonucleotide
cuuacgcugaguacuucgauu

SEQ ID NO: 22 - RNA, 21 nt, Artificial Sequence
  Description of Artificial Sequence siRNA, Synthetic Oligonucleotide
ucgaaguacucagcguaaguu

SEQ ID NO: 23 - RNA, 21 nt, Artificial Sequence
  Description of Artificial Sequence siRNA, Synthetic Oligonucleotide
cuuacgcugaguacuucgauu

SEQ ID NO: 24 - RNA, 21 nt, Artificial Sequence
  Description of Artificial Sequence siRNA, Synthetic Oligonucleotide
ucgaaguacucagcguaaguu

SEQ ID NO: 25 - RNA, 21 nt, Artificial Sequence
  Description of Artificial Sequence siRNA, Synthetic Oligonucleotide
cuuacgcugaguacuucgauu

SEQ ID NO: 26 - RNA, 21 nt, Artificial Sequence
  Description of Artificial Sequence siRNA, Synthetic Oligonucleotide
ucgaaguacucagcguaaguu

SEQ ID NO: 27 - DNA, 21 bp, Artificial Sequence
  Description of Artificial Sequence Combined DNA/RNA Molecule, Synthetic Oligonucleotide
cuuacgcugaguacuucgatn

SEQ ID NO: 28 - DNA, 21 bp, Artificial Sequence
  Description of Artificial Sequence Combined DNA/RNA Molecule, Synthetic Oligonucleotide
ucgaaguacucagcguaagtn

SEQ ID NO: 29 - DNA, 21 bp, Artificial Sequence
  Description of Artificial Sequence Combined DNA/RNA Molecule, Synthetic Oligonucleotide
cuuacgcugaguacuucgann

SEQ ID NO: 30 - DNA, 21 bp, Artificial Sequence
  Description of Artificial Sequence Combined DNA/RNA Molecule, Synthetic Oligonucleotide
ucgaaguacucagcguaagnn

SEQ ID NO: 31 - DNA, 21 bp, Artificial Sequence
  Description of Artificial Sequence Combined DNA/RNA Molecule, Synthetic Oligonucleotide
cuuacgcugaguacuucgatt

SEQ ID NO: 32 - DNA, 21 bp, Artificial Sequence
  Description of Artificial Sequence Combined DNA/RNA Molecule, Synthetic Oligonucleotide
ucgaaguacucagcguaagtt

SEQ ID NO: 33 - DNA, 21 bp, Artificial Sequence
  Description of Artificial Sequence Combined DNA/RNA Molecule, Synthetic Oligonucleotide
cunacgcugaguacuucgauu

SEQ ID NO: 34 - DNA, 21 bp, Artificial Sequence
  Description of Artificial Sequence Combined DNA/RNA Molecule, Synthetic Oligonucleotide
ucgaaguacucagcgnaaguu

SEQ ID NO: 35 - DNA, 21 bp, Artificial Sequence
  Description of Artificial Sequence Combined DNA/RNA Molecule, Synthetic Oligonucleotide
cunacgcugagnacuucgauu

SEQ ID NO: 36 - DNA, 21 bp, Artificial Sequence
  Description of Artificial Sequence Combined DNA/RNA Molecule, Synthetic Oligonucleotide
ucgaagnacucagcgnaaguu

SEQ ID NO: 37 - DNA, 21 bp, Artificial Sequence
  Description of Artificial Sequence Combined DNA/RNA Molecule, Synthetic Oligonucleotide
cuuacgcugaguacuucgatt

SEQ ID NO: 38 - DNA, 21 bp, Artificial Sequence
  Description of Artificial Sequence Combined DNA/RNA Molecule, Synthetic Oligonucleotide
ucgaaguacucagcguaagtt

SEQ ID NO: 39 - DNA, 21 bp, Artificial Sequence
  Description of Artificial Sequence Combined DNA/RNA Molecule, Synthetic Oligonucleotide
cuuacgcugaguacuucgatt

SEQ ID NO: 40 - DNA, 21 bp, Artificial Sequence
  Description of Artificial Sequence Combined DNA/RNA Molecule, Synthetic Oligonucleotide
ucgaaguacucagcguaagtt

SEQ ID NO: 41 - RNA, 21 nt, Artificial Sequence
  Description of Artificial Sequence siRNA, Synthetic Oligonucleotide
gauugauugaccuguccauuc

SEQ ID NO: 42 - RNA, 23 nt, Artificial Sequence
  Description of Artificial Sequence siRNA, Synthetic Oligonucleotide
gaauggacaggucaaucaaucuu

SEQ ID NO: 43 - RNA, 21 nt, Artificial Sequence
  Description of Artificial Sequence siRNA, Synthetic Oligonucleotide
gauugauugaccuguccauuc

SEQ ID NO: 44 - RNA, 23 nt, Artificial Sequence
  Description of Artificial Sequence siRNA, Synthetic Oligonucleotide
gaauggacaggucaaucaaucuu

SEQ ID NO: 45 - RNA, 21 nt, Artificial Sequence
  Description of Artificial Sequence siRNA, Synthetic Oligonucleotide
caccaacuucuuccacgaguc

SEQ ID NO: 46 - RNA, 23 nt, Artificial Sequence
  Description of Artificial Sequence siRNA, Synthetic Oligonucleotide
gacucguggaagaaguugguguu

SEQ ID NO: 47 - RNA, 21 nt, Artificial Sequence
  Description of Artificial Sequence siRNA, Synthetic Oligonucleotide
caccaacuucuuccacgaguc

SEQ ID NO: 48 - RNA, 23 nt, Artificial Sequence
  Description of Artificial Sequence siRNA, Synthetic Oligonucleotide
gacucguggaagaaguugguguu

SEQ ID NO: 49 - RNA, 21 nt, Artificial Sequence
  Description of Artificial Sequence siRNA, Synthetic Oligonucleotide
gaguuugugacaaauaugggc

SEQ ID NO: 50 - RNA, 23 nt, Artificial Sequence
  Description of Artificial Sequence siRNA, Synthetic Oligonucleotide
gcccauauuugucacaaacucca

SEQ ID NO: 51 - RNA, 21 nt, Artificial Sequence
  Description of Artificial Sequence siRNA, Synthetic Oligonucleotide
gaguuugugacaaauaugggc

SEQ ID NO: 52 - RNA, 23 nt, Artificial Sequence
  Description of Artificial Sequence siRNA, Synthetic Oligonucleotide
gcccauauuugucacaaacucca

SEQ ID NO: 53 - RNA, 21 nt, Artificial Sequence
  Description of Artificial Sequence siRNA, Synthetic Oligonucleotide
cuuuacaagccuugguucagu

SEQ ID NO: 54 - RNA, 23 nt, Artificial Sequence
  Description of Artificial Sequence siRNA, Synthetic Oligonucleotide
acugaaccaaggcuuguaaagug

SEQ ID NO: 55 - RNA, 21 nt, Artificial Sequence
  Description of Artificial Sequence siRNA, Synthetic Oligonucleotide
cuuuacaagccuugguucagu

SEQ ID NO: 56 - RNA, 23 nt, Artificial Sequence
  Description of Artificial Sequence siRNA, Synthetic Oligonucleotide
acugaaccaaggcuuguaaagug

SEQ ID NO: 57 - RNA, 21 nt, Artificial Sequence
  Description of Artificial Sequence siRNA, Synthetic Oligonucleotide
ggaaucuuauauuugauccaa

SEQ ID NO: 58 - RNA, 23 nt, Artificial Sequence
  Description of Artificial Sequence siRNA, Synthetic Oligonucleotide
uuggaucaaauauaagauucccu

SEQ ID NO: 59 - RNA, 21 nt, Artificial Sequence
  Description of Artificial Sequence siRNA, Synthetic Oligonucleotide
ggaaucuuauauuugauccaa

SEQ ID NO: 60 - RNA, 23 nt, Artificial Sequence
  Description of Artificial Sequence siRNA, Synthetic Oligonucleotide
uuggaucaaauauaagauucccu

SEQ ID NO: 61 - RNA, 21 nt, Artificial Sequence
  Description of Artificial Sequence siRNA, Synthetic Oligonucleotide
gauugauugaccuguccauuc

SEQ ID NO: 62 - RNA, 23 nt, Artificial Sequence
  Description of Artificial Sequence siRNA, Synthetic Oligonucleotide
gaauggacaggucaaucaaucuu

SEQ ID NO: 63 - RNA, 21 nt, Artificial Sequence
  Description of Artificial Sequence siRNA, Synthetic Oligonucleotide
gauugauugaccuguccauuc

SEQ ID NO: 64 - RNA, 23 nt, Artificial Sequence
  Description of Artificial Sequence siRNA, Synthetic Oligonucleotide
gaauggacaggucaaucaaucuu

SEQ ID NO: 65 - RNA, 21 nt, Artificial Sequence
  Description of Artificial Sequence siRNA, Synthetic Oligonucleotide
caccaacuucuuccacgaguc

SEQ ID NO: 66 - RNA, 23 nt, Artificial Sequence
  Description of Artificial Sequence siRNA, Synthetic Oligonucleotide
gacucguggaagaaguugguguu

SEQ ID NO: 67 - RNA, 21 nt, Artificial Sequence
  Description of Artificial Sequence siRNA, Synthetic Oligonucleotide
caccaacuucuuccacgaguc

SEQ ID NO: 68 - RNA, 23 nt, Artificial Sequence
  Description of Artificial Sequence siRNA, Synthetic Oligonucleotide
gacucguggaagaaguugguguu

SEQ ID NO: 69 - RNA, 21 nt, Artificial Sequence
  Description of Artificial Sequence siRNA, Synthetic Oligonucleotide
gaguuugugacaaauaugggc

SEQ ID NO: 70 - RNA, 23 nt, Artificial Sequence
  Description of Artificial Sequence siRNA, Synthetic Oligonucleotide
gcccauauuugucacaaacucca

SEQ ID NO: 71 - RNA, 21 nt, Artificial Sequence
  Description of Artificial Sequence siRNA, Synthetic Oligonucleotide
gaguuugugacaaauaugggc

SEQ ID NO: 72 - RNA, 23 nt, artificial sequence
  Description of Artificial Sequence siRNA, Synthetic Oligonucleotide
gcccauauuugucacaaacucca

SEQ ID NO: 73 - RNA, 21 nt, Artificial Sequence
  Description of Artificial Sequence siRNA, Synthetic Oligonucleotide
cuuuacaagccuugguucagu

SEQ ID NO: 74 - RNA, 23 nt, Artificial Sequence
  Description of Artificial Sequence siRNA, Synthetic Oligonucleotide
acugaaccaaggcuuguaaagug

SEQ ID NO: 75 - RNA, 21 nt, Artificial Sequence
  Description of Artificial Sequence siRNA, Synthetic Oligonucleotide
cuuuacaagccuugguucagu

SEQ ID NO: 76 - RNA, 23 nt, Artificial Sequence
  Description of Artificial Sequence siRNA, Synthetic Oligonucleotide
acugaaccaaggcuuguaaagug

SEQ ID NO: 77 - RNA, 21 nt, Artificial Sequence
  Description of Artificial Sequence siRNA, Synthetic Oligonucleotide
ggaaucuuauauuugauccaa

SEQ ID NO: 78 - RNA, 23 nt, Artificial Sequence
  Description of Artificial Sequence siRNA, Synthetic Oligonucleotide
uuggaucaaauauaagauucccu

SEQ ID NO: 79 - RNA, 21 nt, Artificial Sequence
  Description of Artificial Sequence siRNA, Synthetic Oligonucleotide
ggaaucuuauauuugauccaa

SEQ ID NO: 80 - RNA, 23 nt, Artificial Sequence
  Description of Artificial Sequence siRNA, Synthetic Oligonucleotide
uuggaucaaauauaagauucccu

SEQ ID NO: 81 - RNA, 21 nt, Artificial Sequence
  Description of Artificial Sequence siRNA, Synthetic Oligonucleotide
gucaucacacugaauaccaau

SEQ ID NO: 82 - RNA, 23 nt, Artificial Sequence
  Description of Artificial Sequence siRNA, Synthetic Oligonucleotide
auugguauucagugugaugacac

(19) United States
(12) Patent Application Publication
(10) Pub. No.: US 2011/0166198 A1
(43) Pub. Date: Jul. 7, 2011

(54) RNA COMPOSITIONS FOR MODULATING IMMUNE RESPONSE

(75) Inventor: Muthiah Manoharan, Cambridge, MA (US)

(73) Assignee: ALNYLAM PHARMACEUTICALS, INC., Cambridge, MA (US)

(21) Appl. No.: 12/936,935

(22) PCT Filed: Apr. 8, 2009
(86) PCT No.: PCT/US2009/039937

§ 371 Date: Mar. 23, 2011

Related U.S. Application Data
(60) Provisional application No. 61/043,883, filed on Apr. 10, 2008.

(51) Int. Cl. A61K 31/7088; C12Q 1/68; G01N 33/53; A61K 31/713; A61P 37/04

(52) U.S. Cl. 514/44 A; 435/6.1; 436/501

(57) ABSTRACT

This invention features modified iRNA agents that modulate an immune response, methods of making and identifying iRNA agents that modulate an immune response, and methods of using the iRNA agents to modulate an immune response.

### RELATED APPLICATION

This application claims priority to PCT Application No. PCT/US09/39937, filed Apr. 8, 2009, which claims priority to U.S. Provisional Application No. 61/043,883, filed Apr. 10, 2008, both of which are herein incorporated by reference in their entirety.

### TECHNICAL FIELD

This invention relates to iRNA and RNA agents that are modified so as to either stimulate or inhibit the immune system of a subject. This invention describes the use of iRNA and RNA agents that bind to immune-related proteins, for example TLR 3, TLR 7, TLR 8 and TLR 9, for modulating their activities. This invention also relates to the use of iRNA agents that sequence-specifically target the mRNA of certain immune-related proteins, for example TLR 3, TLR 7, TLR 8 and TLR 9, for degradation via a RISC-based mechanism. The invention also relates to methods of making and using such modified iRNA agents.

### BACKGROUND

Double-stranded RNA molecules (dsRNA) can block gene expression by virtue of a highly conserved regulatory mechanism known as RNA interference (RNAi). Briefly, the RNA III Dicer enzyme processes dsRNA into small interfering RNA (siRNA) of approximately 22 nucleotides. The siRNA is assembly into a protein/RNA complex called RNA-induced silencing complex (RISC; Bartel, Cell, 2004, 116, 281-297). One of the strands of the siRNA duplex, the antisense strand or guide strand, hybridizes to a messenger RNA (mRNA) through formation of the specific Watson-Crick base pairs. The endonuclease component of the RISC, Slicer, cleaves the targeted mRNA (Meister et al., Mol. Cell, 2004, 15, 185-197). The antisense strand of the siRNA duplex is not cleaved or otherwise degraded in this process, and the RISC that includes this antisense strand can subsequently cleave additional complementary mRNAs.

Many diseases (e.g., cancers, hematopoietic disorders, endocrine disorders, and immune disorders) arise from the abnormal or otherwise unwanted expression or activity of a particular gene or group of genes. For example, disease can result through misregulated gene expression, expression of a mutant form of a protein, or expression of viral, bacterial or other pathogen-derived genes. The RNAi pathway can be used to inhibit or decrease the unwanted expression of such genes (Agrawal et al., Microbiol Mol Biol Rev., 2003, 67, 657-685; Alisky & Davidson, Am. J. Pharmacogenomics, 2004, 4, 45-51).

The iRNA agents described in this patent are duplexes of chemically synthesized oligoribonucleotides. One or both strands of the duplex may be chemically modified to alter the properties of the duplex as described below. In addition to RISC-mediated cleavage of mRNA recognized in a sequence specific manner, the duplexes may have an effect on the immune system. This invention relates to iRNA agents that are modified so as to either stimulate or inhibit the immune system of a subject. These iRNA agents may have a dual function and also sequence-specifically target an mRNA of a gene not related to the immune cascade. These immune stimulatory or inhibitory iRNA agents may also be used in conjunction with a second therapeutic, which may be a chemotherapeutic agent, an antibiotic, or a second iRNA agent. This invention also relates to the use of iRNA agents that sequence specifically target the mRNAs of certain immune-related proteins, for example TLR 3, TLR 7, TLR 8 and TLR 9, for degradation via a RISC-based mechanism.

Pathogen recognition is mediated by the innate recognition arm of the immune system. Unlike the adaptive immune system, innate immunity does not have the potential for recognition of all possible antigens. Instead, a few highly conserved structures present in many different microorganisms are recognized. The innate immune response is based on the recognition of ligands by pathogen recognition receptors (PRRs) on epithelial and immune cells. The ligands include lipopolysaccharide (LPS) from the gram-negative cell wall, peptidoglycan, lipotechoic acids from the gram-positive cell wall, mannose sugar, bacterial DNA, N-formylmethionine, double-stranded RNA from viruses, and glucans from fungal cell walls. Innate immunity is activated immediately or within several hours after exposure to a recognized ligand.

Cells involved in the innate immune response include phagocytic cells, basophils, mast cells, eosinophils, and natural killer cells (NK cells). There are two functionally different classes of cell surface pattern-recognition receptors on these cells: endocytic pattern-recognition receptors and signaling pattern-recognition receptors. For the purpose of this document, signaling pattern-recognition receptors, in general, and the toll-like receptors, in particular, are most important. The toll-like receptors (TLRs) bind several different molecules of microbial origin including DNA from bacteria and double-stranded RNA (Janssens & Beyaert, Clin. Microbiol. Rev., 2003, 16, 637-646).

When a ligand binds to a TLR, a signal is transmitted to the nucleus and genes coding for the synthesis of intracellular regulatory molecules are expressed (Ulevitch, Nat. Rev. Immunol., 2004, 4, 512-520). TLR signaling relies on adaptor proteins including MyD88, Tollip, Mal, and others. These adaptor proteins activate cellular responses to induce production of inflammatory cytokine production, induce maturation of dendritic cells, and induce production of interferons. The cytokines, in turn, trigger innate immune defenses such as inflammation, fever, and phagocytosis and provide an immediate response against the invading microorganism. TLRs also participate in adoptive immunity by triggering various secondary signals needed for humoral immunity (the production of antibodies) and cell-mediated immunity (the production of cytotoxic T-lymphocytes and additional cytokines).

The iRNA agents described in this document may activate the innate immune system through one of the receptors known to bind nucleic acids or nucleotide analogs. Toll-like receptor 3 (TLR3) is the receptor for double-stranded RNA. TLR3 is expressed on dendritic cells, fibroblasts, macrophages, and epithelial cells (Matsumoto et al., Microbial. Immunol., 2004, 48, 147-154). The adaptor molecule for TLR3 is TICAM-1. Binding of TLR3 to TICAM-1 induces multiple signaling cascades that ultimately lead to production of type I interferons (IFN-αβ, Matsumoto et al., Microbial. Immunol., 2004, 48, 147-154). The interferons are cytokines that induce uninfected cells to produce enzymes capable of degrading RNA thus preventing viral replication. Interferons also activate a variety of cells important to defense including cytoxic T-lymphocytes, macrophages, and NK cells.

Single-stranded RNA recognition is mediated in mouse by Toll-like receptor 7 and in humans by TLR-8. In mice, TLR7 binds to the adaptor MyD88 and leads to activation of IFN-α. Diebold et al. (Science, 2004, 303, 1529-1531) showed that influenza virus RNA, polyuridylic acid, and in vitro synthesized mRNA all induced production of IFN-α in plasmacytoid dendritic cells. Heil et al. (Science, 2004, 303, 1526-1529) showed that guanine- and uridine-rich RNA oligonucleotides of 20 residues with phosphorothioate termini stimulated dendritic cells and macrophages to secrete INF-α and proinflammatory and regulatory cytokines. Using TLR-deficient mice, they showed that mouse TLR-7 and human TLR-8 were responsible for binding to single-stranded RNA. Human TLR-7 is also activated by guanine nucleotide analogs (Lee et al., Proc. Natl. Acad. Sci. USA, 2003, 100, 6646-6651).

DNA from bacteria has stimulatory effects on mammalian immune cells. This response depends on the presence of unmethylated CpG dinucleotides in the bacterial DNA; mammalian DNA has a low frequency of CpG dinucleotides and these are mostly methylated, therefore, mammalian DNA does not have immunostimulatory activity. The cellular response to CpG DNA is mediated by TLR9 (Hemmi et al., Nature, 2000, 408, 740-745). Short DNA oligonucleotides with a CpG motif have immune stimulatory effects that depend on the bases flanking the CpG dinucleotide, on the number and spacing of the CpG motifs, on the presence of poly G sequences in the ODN, and on the ODN backbone (Krieg et al., Nature, 1995, 374, 546-549; Krieg, In: Antisense Drug Technology: Principles, Strategies, and Applications, 2001, ed. Crooke, S. T., pp. 471-516, Marcel Dekker, Inc., New York).

Double-stranded RNA may also activate the innate immune system through interaction with a ubiquitously-expressed serine/threonine protein kinase called PKR. PKR is part of the TLR4 cascade activated by TLR4 binding of bacterial LPS. PKR is induced by interferon and activated by dsRNA, cytokines, growth factors, and stress signals. PKR is autophosphorylated and activated upon binding to dsRNA. Activation results in inhibition of protein synthesis via the phosphorylation of eIF2a and also induces transcription of inflammatory genes by PKR-dependent signaling of the activation of different transcription factors (Williams, Oncogene, 1999, 18, 6112-6420). PKR up-regulates NF-κB expression through phosphorylation of its inhibitor IkB (Kumar et al., Proc. Natl. Acad. Sci. USA, 1994, 91, 6288-6292). As few as 11 base pairs of dsRNA can bind to PKR and induce activity, but maximal activation requires at least 30 base pairs (Manche et al., Mol. Cell. Biol., 1992, 12, 5238-5248; Nanduri et al., EMBO J., 1998, 17, 5458-5465). Interestingly, Kim et al. (Nat. Biotechnol., 2004, 22, 321-325) recently showed that the 5′ triphosphate on siRNAs transcribed using T7 RNA polymerase was responsible for type I IFN induction.

There have been reports that siRNA duplexes are able to trigger an immune response in human cells under certain conditions. Sledz et al (Nat. Cell Biol., 2003, 5, 834-839) reported induction of the interferon system with each of the six different siRNA duplexes tested. Bridge et al. (Nat. Genet., 2003, 34, 263-264) reported that some, but not all, of the hairpin siRNAs delivered using viral vectors induced expression of an interferon-stimulated gene. The concentration of the siRNA duplex is directly correlated with the extent of the immune response. These two reports showed that the siRNA duplexes activated PKR. In contrast, data from Kariko et al. (Cells Tissues Organs, 2004, 177, 132-138) implicated the TLR3 pathway in siRNA induction of innate immunity. Kariko et al. (Cells Tissues Organs, 2004, 177, 132-138) showed that TLR3 activation was concentration dependent, but the requirements for optimal TLR3 activation by dsRNA are presently unknown.

Activation of the innate immune response is advantageous in diseases ranging from viral infections to cancer. Activation of innate immunity by dsRNA and CpG DNA promoted antitumor effects in a mouse model (Whitmore et al., Cancer Res., 2004, 64, 5850-5860). iRNA agents should be potent adjuvants for vaccination against a variety of bacterial and viral pathogens. Such adjuvants should activate innate immunity, which, in turn should shape the adaptive immune response. Some iRNA sequences and modifications may better activate the innate immune response than others. This activation could come via any of the Toll-like receptor pathways that are known to bind nucleic acids: TLR3, the receptor for double-stranded RNA; TLR8, the receptor for single-stranded RNA; TLR9, the receptor with a preference for unmethylated CpG DNA; or PKR, the protein kinase activated by dsRNA. Each of these proteins is known to bind to molecules similar to the iRNA agents described.

There are a number of reasons that suppression of the innate immune system might be advantageous. As stimulation of the innate immune system results in inflammation, depression of this system when it is non-productive may benefit patients with asthma, serious local or systemic infections, or chronic inflammatory diseases such as inflammatory bowel disease, chronic obstructive pulmonary disease, and arthritis. Compounds that suppress the immune system may also be useful in treatment of cancer. In particular, disruption of the PKR pathway, which leads to activation of NF-κB, is attractive. NF-κB leads to inhibition of apoptosis that would otherwise eliminate defective cells (Pikarsky et al., Nature, 2004, 431, 461-466) and also promotes metastatic growth (Huber et al., J. Clin. Invest., 2004, 114, 569-581).

### SUMMARY

The invention features iRNA (interfering RNA) agents that are modified so as to have an effect, or to specifically avoid having an effect, on the immune system of a subject. The modified iRNA agents may 1) stimulate the immune system of a subject; 2) inhibit the immune system of a subject; 3) have no direct effect on the immune system, e.g., an iRNA agent that has been designed to not interact with a cytokine or TLR protein, or 4) have a dual function and effect the immune system through binding to a protein involved in the immune cascade and also sequence-specifically target an mRNA of a gene not related to the immune system; or 5) sequence-specifically target the mRNA of certain immune-related proteins, for example TLR 3, TLR 7, TLR 8 and TLR 9, for RNA interference-based mechanisms including degradation via a RISC-based mechanism. The invention features methods of making and identifying iRNA agents having such properties, as well as compositions and methods for modulating an immune response by using the iRNA agent. While not wishing to be bound by theory, it is believed that modulation of immune system activity can result from an interaction of the iRNA agent with a component of the immune system, where the interaction disrupts or stimulates an activity of the component. This is independent of RISC-mediated gene silencing and is believed to act through an interaction of single-stranded or double-stranded RNA with a protein component of the immune system. The iRNA agents featured in the invention are called immunoselective iRNA agents (it is understood that this term includes iRNA agents modified or selected to have an effect on the immune system or specifically designed or selected to have no effect on the immune system).

Without being bound by theory, it is believed that an iRNA agent, e.g., an unmodified iRNA agent, may stimulate or inhibit an immune response, e.g., by interacting with one or more protein components of the immune system, such as a growth factor (e.g. cytokine or interleukin) or a cell surface receptor protein (e.g., a toll-like receptor proteins, such as TLR 3, TLR 7, TLR 8 and TLR 9, Toll-like receptor 7 or TLR7 and Toll-like receptor 9 or TLR9). Additionally, the unmodified iRNA agent may stimulate the immune system when the two strands of the iRNA agent separate in a biological system (e.g., in a tissue or fluid, such as the blood stream) before the agent reaches its target tissue.

An immune system response that is effectively prevented is a response that is weaker than a response triggered by an iRNA agent that does not include the particular modification(s). An immune system response that is effectively stimulated is a response that is stronger than the response triggered by an iRNA agent that does not include the particular modifications. Methods of measuring an immune response are described below.

In one aspect, the invention relates to methods of identifying an immunoselective iRNA agent that exhibits a preselected level of interaction with the immune system of a subject, e.g., it stimulates (or inhibits) the immune system, or does not affect the immune system. The method includes providing a candidate iRNA agent that includes a modified sense and/or antisense strand; and evaluating the modified strand, both strands, or the modified strand annealed to its complement, for the ability to modulate the immune system.

Methods of identifying an immunoselective iRNA agent include evaluating the modified antisense and/or sense strands for an ability to modulate the immune system. Modulation of the immune system can be measured for example by (i) measurement of either the mRNA or protein expression levels of a component (e.g., a growth factor, cytokine, or interleukin) of the immune system, e.g., in a cell or in an animal, (ii) measurement of the mRNA or protein levels of a protein factor activated by a component of the immune system (for example, NFKB), e.g., in a cell or in an animal, (iii) measurement of cell proliferation, e.g., in a tissue explant or a tissue of an animal.

Evaluation of a candidate immunoselective iRNA agent can include incubating the modified strand (with or without its complement, but preferably annealed to its complement) with a biological system, e.g., a sample (e.g, a cell culture). The biological sample can be capable of expressing a component of the immune system. This allows identification of an iRNA agent that has an effect on the component. In one embodiment, the step of evaluating whether the iRNA agent modulates, e.g, stimulates or inhibits, an immune response includes evaluating expression of one or more growth factors, such as a cytokine or interleukin, or cell surface receptor protein, in a cell free, cell-based, or animal assay. Exemplary assay methods include ELISA and Western blot analysis. Growth factors that could be evaluated include TNFα, IL1α and β, IL2, IL3, IL4, IL5, IL6, IL7, IL8, IL9, IL10, IL11, IL12, IL13, IFNα and β, and IFNγ. In preferred embodiments, a test includes evaluating expression of one or more of the interleukins IL-10, IL-12, and IL-6. Relevant cell surface receptors include the toll-like receptors, e.g., TLR1, TLR2, TLR3, TLR4, TLR5, TLR6, TLR7, TLR8, or TLR9. In other preferred embodiments, a test includes evaluating expression of one or more of the toll-like receptors TL-3, TLR7, TLR8, or TLR9. Ligand interaction with TLR9 stimulates expression of NFκB. Therefore, testing whether an iRNA agent stimulates the immune response can include assaying for NFκB protein or mRNA expression.

In one embodiment, the step of testing whether the modified iRNA agent modulates, e.g., stimulates, an immune response includes assaying for an interaction between the iRNA agent and a protein component of the immune system, e.g., a growth factor, such as a cytokine or interleukin, or a cell surface receptor protein. For example, the test can include assaying for an interaction between the modified iRNA and a toll-like receptor, e.g., TLR1, TLR2, TLR3, TLR4, TLR5, TLR6, TLR7, TLR8, or TLR9. In one preferred embodiment, testing includes assaying for an interaction with a toll-like receptor, e.g., TLR-9. Exemplary assay methods include coimmunoprecipitation assays, bead-based co-isolation methods, nucleic acid footprint assays and colocalization experiments such as those facilitated by immunocytochemistry techniques.

In one embodiment, the candidate iRNA agent has the ability to sequence-specifically inhibit expression of a particular gene through the RNA interference pathway. For example, the iRNA agent can target a sequence that encodes a protein component of the immune system, such as a cytokine, cytokine receptor or Toll-like receptor (e.g., TLR 3, TLR 7, TLR 8 and TLR 9). The ability of an iRNA agent composition of the invention to inhibit gene expression can be measured using a variety of techniques including Northern blot analysis or RT-PCR for the specific mRNA or Western blot analysis for the amount of target protein present. In still another embodiment, a phenotype influenced by the amount of the protein can be detected.

In one embodiment, the candidate iRNA agent interacts with a protein component of the immune system, or has been modified not to interact, and also targets a sequence that is not involved in the immune system.

In one embodiment, the candidate iRNA agent includes at least one ribonucleotide modification (e.g., as described below), and the presence of the modification modulates an immunostimulatory response (e.g., as compared to what could be seen with an iRNA agent lacking the modification) when the modified iRNA agent is contacted with a cell or administered to a subject. In preferred embodiments, one or more natural bases of an iRNA agent are replaced by modified bases. In more preferred embodiments, one or more natural bases of an iRNA agent are replaced by modified bases. Particular modifications to the iRNA agent may prevent the strands of the agent from separating and subsequently interacting with the one or more protein components of the immune system. Particular modifications may also inhibit the interaction of a double stranded or single stranded iRNA agent from interacting with a protein component of the immune system to an extent that an immune system response is effectively prevented.

Chemical modifications can include modifications to the nucleotide base, the sugar, or the backbone. In one embodiment, a candidate immunoselective iRNA agent includes a substitution of an adenine with a 2-substituted purine (e.g., 2-amino-adenine), a 6-substituted purine, a 7-deaza-alkyl-substituted purine, a 7-deaza-alkenyl-substituted purine, a 7-deaza-alkynyl-substituted purine, or a purine that is not adenine (e.g., guanine or inosine). In another embodiment, the candidate iRNA agent includes a substitution of a guanine with an inosine, an aminopurine, a 2-substituted guanine, a 7-deaza-alkyl-substituted guanine, a 7-deaza-alkenyl-substituted guanine, a 7-deaza-alkynyl-substituted guanine, or an O-6-alkylated guanine. In another embodiment, the candidate iRNA agent includes a substitution of a cytosine with a 5-substituted cytosine (e.g., a 5-methyl cytosine), an N-4 substituted cytosine, a G-clamp, an analog of a G-clamp, a 2-thio-cytosine, a 4-thio-cytosine, or a uracil. In one embodiment, the candidate iRNA agent includes a substitution of a uracil with a 5-substituted uracil, a 4-thio-uracil, a 5-methyl-2-thio-uracil, a pseudouridine, a 1-alkylpseudouridine, a 3-alkylpseudouridine or a 2-thio-uracil. In one embodiment, the iRNA agent includes a 2′-deoxyfluoro, 2′-O-methyl, 2′-O-methoxyethyl, 2′-O-alkyl, 2′-O-alkoxyalkyl, 2′-O-allyl, 2′-O-propyl, 2′-O—(N-methyl-acetamide (NMA), 2′-O—(N,N-dimethylaminooxyethyl), or G-clamp modification. In one embodiment, the iRNA agent includes an arabinose-containing nucleoside that replaces a ribonucleoside. In another embodiment, the arabinose-containing nucleoside can be a 2′-fluoroarabinose-containing nucleoside, or a 2′-O-methylarabinose-containing nucleoside. In another embodiment, the iRNA agent includes a deoxynucleoside that replaces a ribonucleoside. In one embodiment, the deoxynucleoside is a 2′-fluorodeoxynucleoside, or a 2′-O-methyldeoxynucleoside.

In one embodiment, an immunoselective iRNA agent includes at least one backbone modification, e.g., a phosphorothioate, boronaphosphate, methylphosphonate or dithioate modification. In another embodiment, the iRNA agent includes a P-alkyl modification in the linkages between one or more of the terminal nucleotides of an iRNA agent. In another embodiment, the sense and/or antisense strand is substantially free of stereogenic phosphorus atoms having an Rp configuration, and in another embodiment, the sense and/or antisense strand is substantially free of stereogenic phosphorus atoms having an Sp configuration.

In another embodiment, one or more terminal nucleotides of an iRNA agent include a sugar modification, e.g., a 2′ or 3′ sugar modification. In one embodiment, the iRNA agent includes at least two sugar 2′ modifications. Exemplary sugar modifications include, for example, a 2′-fluoro nucleotide, a 2′-O-alkyl nucleotide, a 2′-O-alkoxyalkyl nucleotide, a 2′-O-allyl nucleotide, a 2′-O-propyl nucleotide, a 2′-O-methylated nucleotide (2′-O-Me), a 2′-deoxy nucleotide, a 2′-deoxyfluoro nucleotide, a 2′-O-methoxyethyl nucleotide (2′-O-MOE), a 2′-O—N-MeAcetamide nucleotide (2′-O-NMA), a 2′-O-dimethylaminoethyloxyethyl nucleotide (2′-O— DMAEOE), a 2′-aminopropyl, a 2′-hydroxy, a 2′-ara-fluoro, or 3′-amidate (3′—NH in place of 3′-O), a locked nucleic acid (LNA), extended ethylene nucleic acid (ENA), hexose nucleic acid (HNA), or cyclohexene nucleic acid (CeNA).

In one embodiment, the candidate immunoselective iRNA agent includes a 3′ sugar modification, e.g., a 3′-O-Me modification. Preferably a 3′-O-Me modification is on the sense strand of the iRNA agent.

In some embodiments, a candidate immunoselective iRNA agent includes a 5′-alkyl-pyrimidine e.g 5′-methyl-pyrimidine (e.g., a 5′-methyl-uridine modification or a 5′-methyl-cytodine modification).

The modifications described herein can be combined onto a single candidate iRNA agent. For example, in one embodiment, at least one nucleotide of an iRNA agent has a phosphorothioate linkage and at least one nucleotide has a 2′ sugar modification, e.g., a 2′-β-Me or 2′-deoxyfluoro modification. In another embodiment, at least one nucleotide of a candidate iRNA agent has a 5′-Me-pyrimidine and a 2′ sugar modification, e.g., a 2′-deoxyfluoro or 2′-O-Me modification.

In one embodiment, a candidate immunoselective iRNA agent includes a nucleobase modification, such as a cationic modification, such as a 3′-abasic cationic modification. The cationic modification can be e.g., an alkylamino-dT (e.g., a C6 amino-dT), an allylamino conjugate, a pyrrolidine conjugate, a pthalamido, a porphyrin, or a hydroxyprolinol conjugate.

In one embodiment, the iRNA agent includes at least one ribonucleotide modification on the sense strand and at least one ribonucleotide modification on the antisense strand, and the ribonucleotide modifications on the two strands are different.

In another embodiment, a candidate immunoselective iRNA agent, e.g., an iRNA agent that can inhibit an immune response, may include a conjugate on one or more nucleotides of the iRNA agent. The conjugate can be, for example, a lipophile, a terpene, a protein-binding agent, a vitamin, a carbohydrate, or a peptide. For example, the conjugate can be naproxen, nitroindole (or another conjugate that contributes to stacking interactions), folate, ibuprofen, or a C5 pyrimidine linker. In other embodiments, the conjugates are glyceride lipid conjugates (e.g., a dialkyl glyceride derivatives), vitamin E conjugates, or thio-cholesterols.

In one embodiment, the conjugate is cholesterol, and the cholesterol is conjugated to the sense strand of the iRNA agent, e.g., by a pyrrolidine linker, serinol linker, or hydroxyprolinol linker. In other embodiments, the conjugate is a dU-cholesterol, or cholesterol is conjugated to the iRNA agent by a disulfide linkage. In another embodiment, the conjugate is cholanic acid, and the cholanic acid is attached to the sense strand or the antisense strand. In one embodiment, the cholanic acid is attached to the sense strand and the antisense.

In another embodiment, one or more nucleotides have a 2′-5′ linkage, and preferably, the 2′-5′ linkage is on the sense strand.

In one embodiment, the candidate immunoselective iRNA agent includes an L-sugar, preferably on the sense strand.

In one embodiment, the candidate immunoselective iRNA agent includes a methylphosphonate.

In one embodiment, the candidate immunoselective iRNA agent has been modified by replacing one or more ribonucleotides with deoxyribonucleotides. Preferably, adjacent deoxyribonucleotides are joined by phosphorothioate linkages, and the iRNA agent does not include more than four consecutive deoxyribonucleotides on the sense or the antisense strands.

In some embodiments, the candidate immunoselective iRNA agent includes a difluorotoluoyl (DFT) modification, e.g., 2,4-difluorotoluoyl uracil, or a guanidine to inosine substitution.

In one embodiment, the candidate immunoselective iRNA agent includes a 5′-uridine-adenine-3′ (5′-UA-3′) dinucleotide wherein the uridine is a 2′-modified nucleotide, or a 5′-uridine-guanine-3′ (5′-UG-3′) dinucleotide, wherein the 5′-uridine is a 2′-modified nucleotide, or a 5′-cytidine-adenine-3′ (5′-CA-3′) dinucleotide, wherein the 5′-cytidine is a 2′-modified nucleotide, or a 5′-uridine-uridine-3′ (5′-UU-3′) dinucleotide, wherein the 5′-uridine is a 2′-modified nucleotide, or a 5′-cytidine-cytidine-3′ (5′-CC-3′) dinucleotide, wherein the 5′-cytidine is a 2′-modified nucleotide, or a 5′-cytidine-uridine-3′ (5′-CU-3′) dinucleotide, wherein the 5′-cytidine is a 2′-modified nucleotide, or a 5′-uridine-cytidine-3′ (5′-UC-3′) dinucleotide, wherein the 5′-uridine is a 2′-modified nucleotide. The chemically modified nucleotide in the iRNA agent may be a 2′-O-methylated nucleotide. In some embodiments, the modified nucleotide can be a 2′-deoxy nucleotide, a 2′-deoxyfluoro nucleotide, a 2′-O-methoxyethyl nucleotide, a 2′-O-NMA, a 2′-DMAEOE, a 2′-aminopropyl, 2′-hydroxy, or a 2′-ara-fluoro, or 3′-amidate (3′—NH in place of 3′-O), a locked nucleic acid (LNA), extended nucleic acid (ENA), hexose nucleic acid (HNA), or cyclohexene nucleic acid (CeNA).

In one embodiment, the candidate immunoselective iRNA agent has a single overhang, e.g., one end of the iRNA agent has a 3′ or 5′ overhang and the other end of the iRNA agent is a blunt end. In another embodiment, the iRNA agent has a double overhang, e.g., both ends of the iRNA agent have a 3′ or 5′ overhang, such as a dinucleotide overhang. In another embodiment, both ends of the iRNA agent have blunt ends.

In one embodiment, the candidate immunoselective iRNA agent includes a sense RNA strand and an antisense RNA strand, and the antisense RNA strand is 18-30 nucleotides in length. In another embodiment, the iRNA agent includes a nucleotide overhang having 1 to 4 unpaired nucleotides, which may be at the 3′-end of the antisense RNA strand, and the nucleotide overhang may have the nucleotide sequence 5′-GC-3′ or 5′-CGC-3′. The unpaired nucleotides may have at least one phosphorothioate dinucleotide linkage, and at least one of the unpaired nucleotides may be chemically modified in the 2′-position. In one embodiment, the double strand region of the candidate iRNA agent includes phosphorothioate linkages on one or both of the sense and antisense strands. In a preferred embodiment, the candidate iRNA agent includes phosphorothioate linkages between nucleotides 1 through 5 of the 5′ or 3′ end of the sense or antisense agent.

In one embodiment, the antisense RNA strand and the sense RNA strand are connected with a linker. The chemical linker may be a hexaethylene glycol linker, a poly-(oxyphosphinico-oxy-1,3-propandiol) linker, an allyl linker, or a polyethylene glycol linker. Use of a linker to connect the antisense and sense strands, will inhibit strand separation in vivo, thereby inhibiting immunostimulation.

In another embodiment, one or more modifications of a candidate immunoselective iRNA agent can increase the ratio of double-stranded to single-stranded iRNA agent in a biological system (e.g., in the blood stream or in serum samples). Such modifications may decrease the dissociation constant (K_{D}) between the sense and antisense strands. In some embodiments, the modification that increases the ratio of double-stranded to single-stranded iRNA agent in a biological system is a chemical linker, such as a hexaethylene glycol linker, poly-(oxyphosphinico-oxy-1,3-propandiol) linker, allyl linker, or polyethylene glycol linker that binds the two strands together. In one embodiment, the linker includes an ester, and the linker can be cleaved by an esterase. In some embodiments, the modification is a high affinity chemical modification, such as 2′-F, LNA, ENA, 2′-O-MOE, and C-5-propynyl pyrimidines or G-clamp and its analogs. In yet other embodiments, the modification is a chemical crosslink, e.g., a disulfide containing crosslink.

In another embodiment, the immunoselective iRNA agent can include at least two modifications. The modifications can differ from one another, and may be applied to different RNA strands of a double-stranded iRNA agent. For example, the sense strand can include at least one modification, and the antisense strand can include a modification that differs from the modification or modifications on the sense strand. In another example, the sense strand can include at least two different modifications, and the antisense strand can include at least one modification that differs from the two different modifications on the sense strand. Accordingly, the sense strand can include multiple different modifications, and the antisense strand can include further multiple modifications, some of which are the same or unique from the modifications on the sense strand.

The candidate immunoselective iRNA agents may be prepared by chemical synthesis.

In one aspect, the invention relates to methods of designing an iRNA agent that binds specifically to one of the protein components of the immune system and either inhibits or stimulates the immune cascade. The method may include designing an iRNA agent that includes a sense strand, and an antisense strand sufficiently complementary to hybridize to the sense strand, designing the strands such that one or more of the nucleotides on the sense and/or antisense strand are modified as described herein. The iRNA agent may be further synthesized and tested in an in vitro or in vivo system for binding to a protein component of the immune system, e.g., as described above. For example, the iRNA agent can be tested by assaying for an interaction with a component of the immune system, e.g., a growth factor, such as a cytokine or interleukin protein, or cell-surface receptor. The assay can include introducing an iRNA agent into a cell, maintaining the cell under conditions suitable for expressing a component of the immune system, and determining whether the iRNA agent can interact with the component of the immune system, e.g., by co-immunoprecipitation experiments, or colocalization experiments, such as those facilitated by immunocytochemistry techniques. The cell may be a mammalian cell. The iRNA agent may be tested for an immune stimulatory or immune inhibitory effect in a cellular assay. The iRNA agent may be tested in an in vivo system by administering the iRNA agent to a mammal, such as a mouse, then examining the spleen for enlargement or increased cell proliferation, or for an increase in production of, for example, one or more interleukin proteins, such as IL10, IL12 and/or IL6.

In some embodiments, the candidate iRNA agent is tested in a first system, e.g., a cell-free system or cell-based system, and then retested. The retest can be in the same or different assays. For example, the same or a different cell-based or cell-free assay can be used to confirm activity, or an animal-based system can be used to confirm activity (e.g., gene-silencing activity, or stimulation or inhibition of the immune system) or lack of activity with respect to its effect on an immune response. In some embodiments, the candidate iRNA agent is tested first in a cell-free or cell-based system and is then retested in an animal-based system.

In one aspect, the invention features an iRNA agent that includes at least one ribonucleotide modification, e.g., a ribonucleotide agent described above, and the presence of the modification inhibits or stimulates an immunostimulatory response (e.g., as compared to what could be seen with an iRNA agent lacking the modification) when the modified iRNA agent is contacted with a cell or administered to a subject, e.g., a mammalian subject, such as a human. In preferred embodiments, one or more natural bases of an iRNA agent are replaced by modified bases.

In one aspect the invention features a method of evaluating an iRNA agent that includes providing a candidate single stranded iRNA agent having at least one ribonucleotide modification; contacting the candidate single stranded iRNA agent to a cell-free system, cell, or animal; and evaluating the immune response in the cell-free system, cell, or animal as compared to an immune response in a cell-free system, cell, or animal that is contacted with an unmodified single stranded iRNA agent. The candidate single stranded iRNA agent stimulates an immune response to a lesser or greater extent than a reference. For example, an unmodified iRNA agent is determined to be an iRNA agent that modulates an immune system response. In one embodiment, the candidate single-stranded iRNA agent is 15-2000 nucleotides in length (e.g., 17, 19, 21, 23, 25, 27, 30, 100, 500, 1000, or 1500 nucleotides in length).

In one aspect, the invention relates to an iRNA agent with dual function: an iRNA agent that either inhibits or stimulates the immune system and also sequence-specifically targets an mRNA of therapeutic relevance for degradation via the RISC-mediated RNA interference pathway. The immunoselective iRNA agent (e.g., an iRNA agent that inhibits or stimulates immunostimulation) is prepared by a process described herein, e.g., a process that includes providing a first oligonucleotide that is sufficiently complementary to a target nucleic acid to anneal to the target and a second oligonucleotide sufficiently complementary to anneal to the first oligonucleotide. The first and/or second oligonucleotides include one or more modified nucleotides or nucleotide linkages described above. One or both of the antisense and sense strands, or preferably annealed antisense and sense strands, is tested, e.g., as described herein, for the ability to modulate, e.g., stimulate or inhibit an immune response, e.g., when administered to a test subject. For example, if the iRNA agent does not stimulate an immune response to a preselected magnitude, e.g., to the magnitude of an iRNA agent lacking the one or more modified nucleotides then the agent is determined to inhibit immunostimulation. The modified iRNA agent, e.g., one determined to inhibit or stimulate immunostimulation, is further tested to determine if it can decrease expression of a target mRNA, e.g., by the mechanism of RNA interference, e.g., to the same extent as the unmodified version of the iRNA agent.

In another embodiment, the invention relates to the silencing of the gene for a particular component of the immune system, e.g. toll-like receptor 7 (TLR7) or toll-like receptor 9 (TLR9). In this embodiment, the iRNA agent is designed to be complementary to a region of the mRNA for the component and the iRNA agent decreases the expression of the target through the mechanism of RNA interference.

In still another aspect, the invention relates to a pharmaceutical composition including an immunoselective iRNA agent, as described above, and a pharmaceutically acceptable carrier. The pharmaceutically acceptable carrier may be an aqueous solution, such as phosphate buffered saline, or it may include a micellar structure, such as a liposome, capsid, capsoid, polymeric nanocapsule, or polymeric microcapsule.

In yet a further aspect, the invention relates to a method for treating a disease or disorder in a subject. The method includes identifying a subject having or at risk for developing the disease, administering a pharmaceutical composition containing an immunoselective iRNA agent having one or more of the modified nucleotides or linkages described above, and a pharmaceutically acceptable carrier. The subject may be monitored for an effect on the immune system, e.g., an immunostimulatory or immunoinhibitory response, such as by monitoring for increased expression of a growth factor, such as a cytokine or a cell-surface receptor (e.g., a Toll-like receptor) as described above. Cytokines of interest can be those expressed from T cells, B cells, monocytes, macrophages, dendritic cells, or natural killer cells of the subject. The assays can be performed using blood or serum samples from the subject. The disease or disorder can be one where it is particularly undesirable to stimulate the immune system, e.g., in a patient that has received organ, tissue or bone marrow transplants. In another alternative, the disease or disorder can be one where it is particularly desirable to stimulate the immune system, e.g., in patients with cancer or viral diseases. In one embodiment, the subject is immunocompromised, and an iRNA agent that includes nucleotide modifications stimulates an immune response in a cell to a greater extent than an iRNA agent that does not include nucleotide modifications. The subject may be a mammal, such as a human.

In a preferred embodiment, administration of an immunoselective iRNA agent is for treatment of a disease or disorder present in the subject. In another preferred embodiment, administration of the iRNA agent is for prophylactic treatment.

In one aspect, the invention features a method of increasing the ratio of double stranded iRNA (dsiRNA) agent to single stranded iRNA (ssiRNA) agent in a human by administering to the human a dsiRNA agent that includes one or more modifications that inhibit disassociation of the dsiRNA agent as compared to a dsiRNA agent that does not include the modifications. In one embodiment, the modifications include a chemical linker, such as a hexaethylene glycol linker, poly(oxyphosphinico-oxy-1,3-propandiol) linker, allyl linker, or polyethylene glycol linker. In another embodiment, the modifications increase the melting temperature of the dsiRNA as compared to a dsiRNA that does not include the modifications. Such modifications can include a locked nucleic acid, G-clamp, 2′-O-methyl, 2′-fluoro, 2′-O-methoxyethyl, 2-thio-pyrimidine, 2-amino-adenine or pseudouridine. In some embodiments, the modifications of the dsiRNA agent occur only in the sense strand of the dsiRNA agent or only in the antisense strand of the dsiRNA agent.

In one aspect, the invention features a method of selecting a patient suitable for treatment with an immunoselective iRNA agent described herein. In one embodiment, the selection of the patient is based on need for either immunostimulation or immunosuppression. In another embodiment, selection is based on the identification of a patient in need of decreased expression of a gene not involved in the immune system, and also in need of increased or decreased immune system function. The patient's need with respect to immune system function will determine which modifications will be incorporated into the therapeutic immunoselective iRNA agent.

In one aspect, the invention features an immunoselective iRNA agent that stimulates the immune system. For example, a stimulatory immunoselective iRNA agent includes one or more modifications, e.g., modifications described herein for stimulating the immune system. For example, the modified iRNA agent will stimulate the immune system to a greater extent than an iRNA agent that does not include the one or more modifications. These stimulatory immunoselective iRNA agents can be administered to immunocompromised patients, or can be administered with a second therapy to off-set immunocompromising effects of the second therapy. The second therapeutic can be a chemotherapy or antibiotic, for example. A stimulatory immunoselective iRNA agent can be administered to a subject having or at risk for developing a tumor, an autoimmune disease, airway inflammation (e.g., asthma), an allergy (e.g., a food or respiratory allergy), or a pathogenic disease, such as a disease caused by a bacteria, fungus, parasite, virus or viroid particle, or prion.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 a general synthetic scheme for incorporation of RRMS monomers into an oligonucleotide.

FIG. 2A is a list of substituents that may be present on silicon in OFG¹.

FIG. 2B is a list of substituents that may be present on the C2′-orthoester group.

FIG. 3 is list of representative RRMS cyclic carriers. Panel 1 shows pyrroline-based RRMSs; panel 2 shows 3-hydroxyproline-based RRMSs; panel 3 shows piperidine-based RRMSs; panel 4 shows morpholine and piperazine-based RRMSs; and panel 5 shows decalin-based RRMSs. R1 is succinate or phosphoramidate and R2 is H or a conjugate ligand.

FIG. 4 is a general reaction scheme for 3′ conjugation of peptide into iRNA.

FIG. 5 is a general reaction scheme for 5′ conjugation of peptide into iRNA.

FIG. 6 is a general reaction scheme for the synthesis of aza-peptides.

FIG. 7 is a general reaction scheme for the synthesis of N-methyl amino acids and peptides.

FIG. 8 is a general reaction scheme for the synthesis of β-methyl amino acids and Ant and Tat peptides.

FIG. 9 is a general reaction scheme for the synthesis of Ant and Tat oligocarbamates.

FIG. 10 is a general reaction scheme for the synthesis of Ant and Tat oligoureas.

FIG. 11 is a schematic representation of peptide carriers.

FIGS. 12A and 12B are bar graphs depicting IFNalpha concentrations in human PBMC cells from two different donors following transfection with the indicated iRNA agents. Transfections were performed with GP2 reagent.

FIGS. 13A and 13B are bar graphs depicting IFNalpha concentrations in human PBMC cells from two different donors following transfection with the indicated iRNA agents. Transfections were performed with Lipofectamine-2000 reagent.

### DETAILED DESCRIPTION

The invention is based, at least in part, on the discovery that particular modifications to an iRNA agent effectively modulate, or avoid a modulation of, an immune response in a subject. Thus, the invention features iRNA agents that contain such modifications and methods of making and using the iRNA agents.

The immunoselective iRNA agents can include one or more of various modifications. For example, the iRNA agent can include a backbone modification to one or more of the terminal nucleotides on an iRNA strand. Exemplary backbone modifications include phosphorothioate linkages or P-alkyl linkages between one or more of the terminal nucleotides of an iRNA agent. One or more terminal nucleotides of an iRNA agent can also include a sugar modification, e.g., a 2′ or 3′ sugar modification. Exemplary sugar modifications are described below. An iRNA agent can include multiple modifications that confer an enhanced resistance to exonucleolytic cleavage. For example, one or more terminal nucleotides of an iRNA agent can have a phosphorothioate linkage and a 2′ sugar modification.

An immunoselective iRNA agent can include a nucleobase modification, a cationic modification on one or more of the terminal nucleotides of the iRNA agent.

Exemplary modifications for modulating the immune response are described below.

Exemplary Bases, 2′-Substituents and Backbones

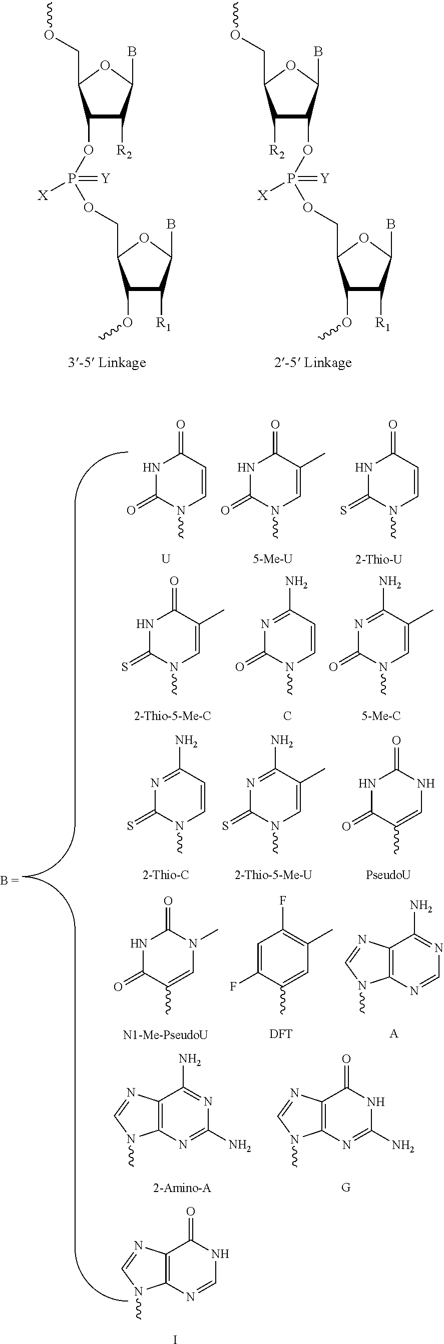

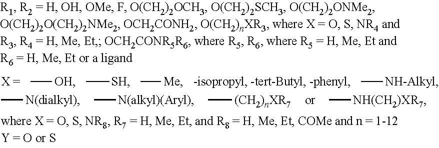

Most Preferred Bases, 2′-Substituents and Backbones with α-Nucleosides

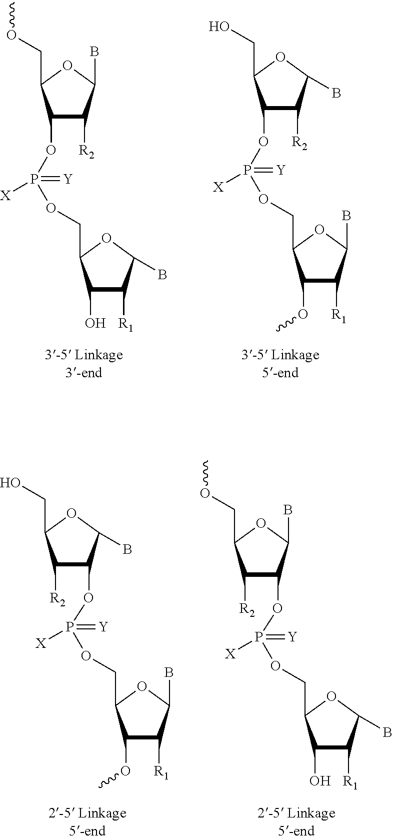

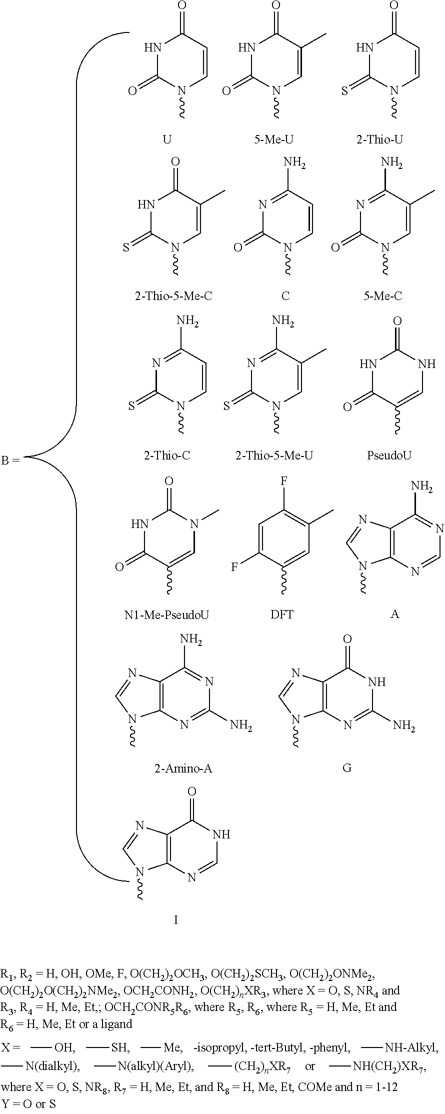

Most Preferred Bases, 2′-Substituents and Backbones with L-Nucleoside

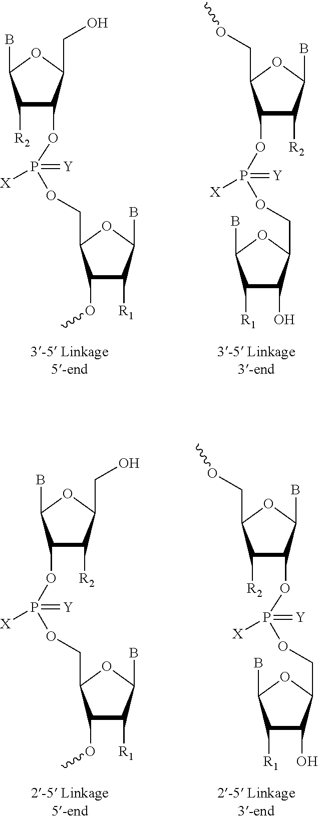

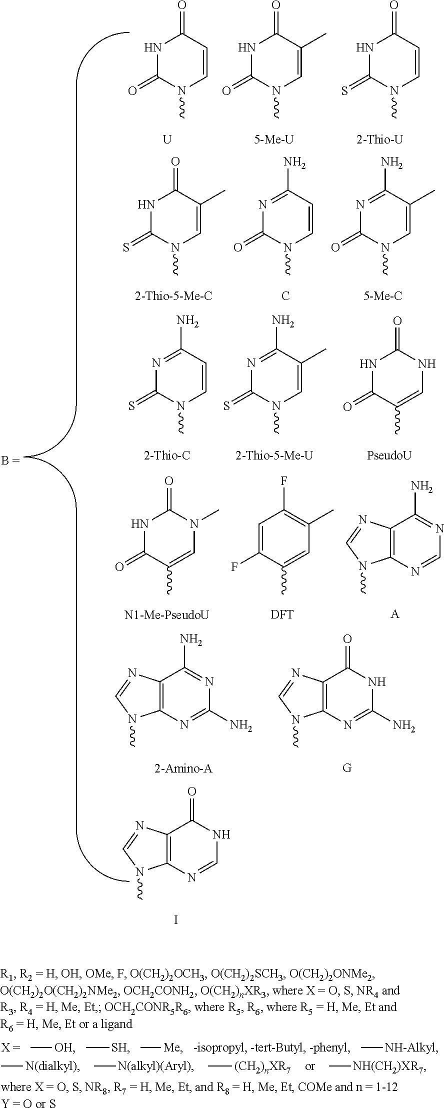

Most Preferred Sugar with 2′-Substituents and Backbones

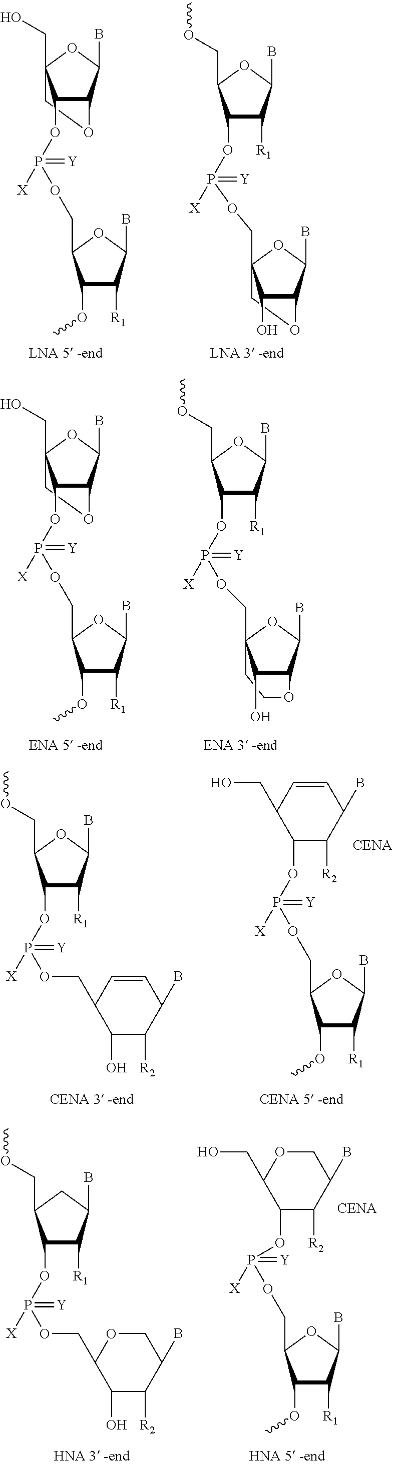

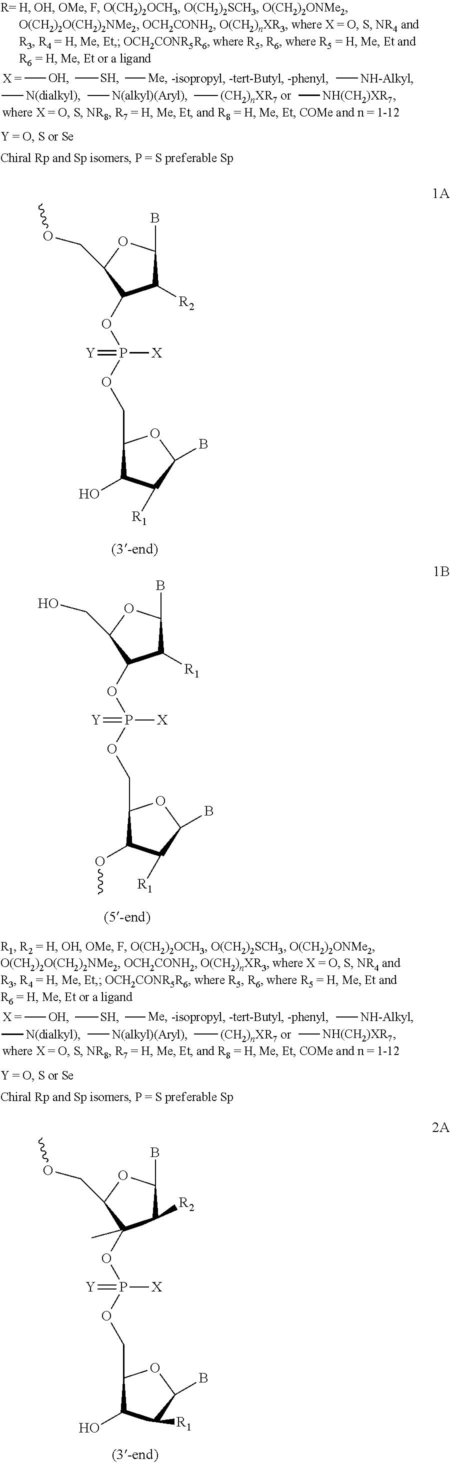

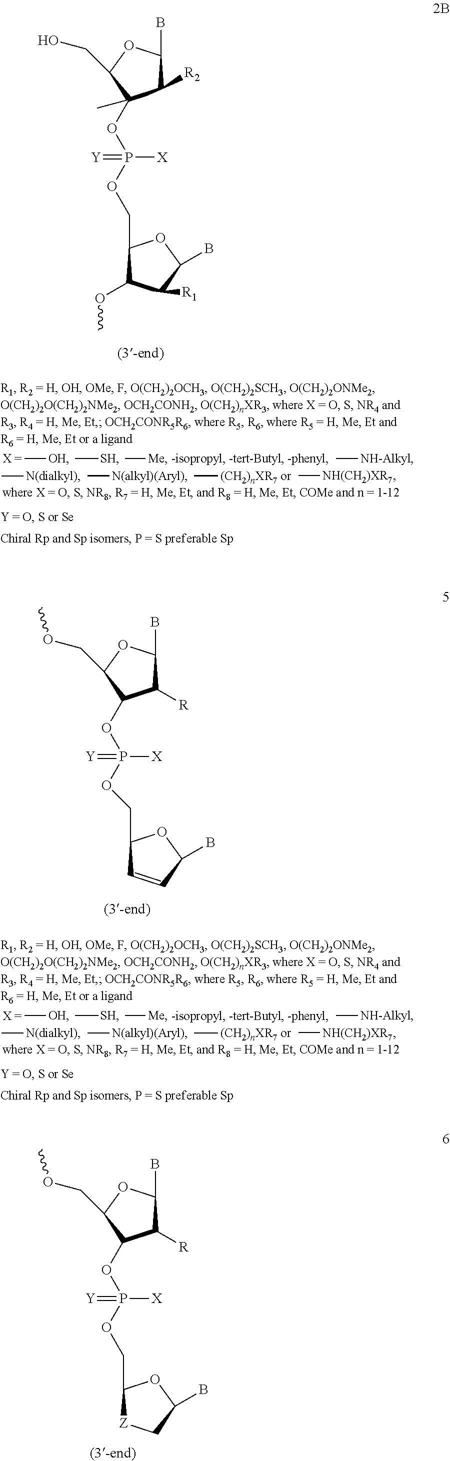

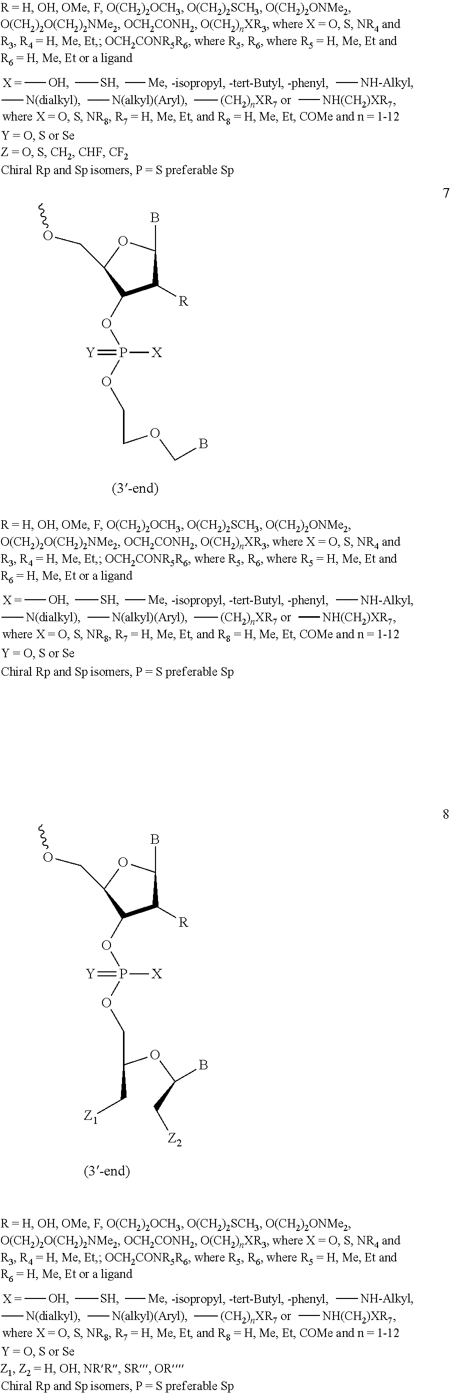

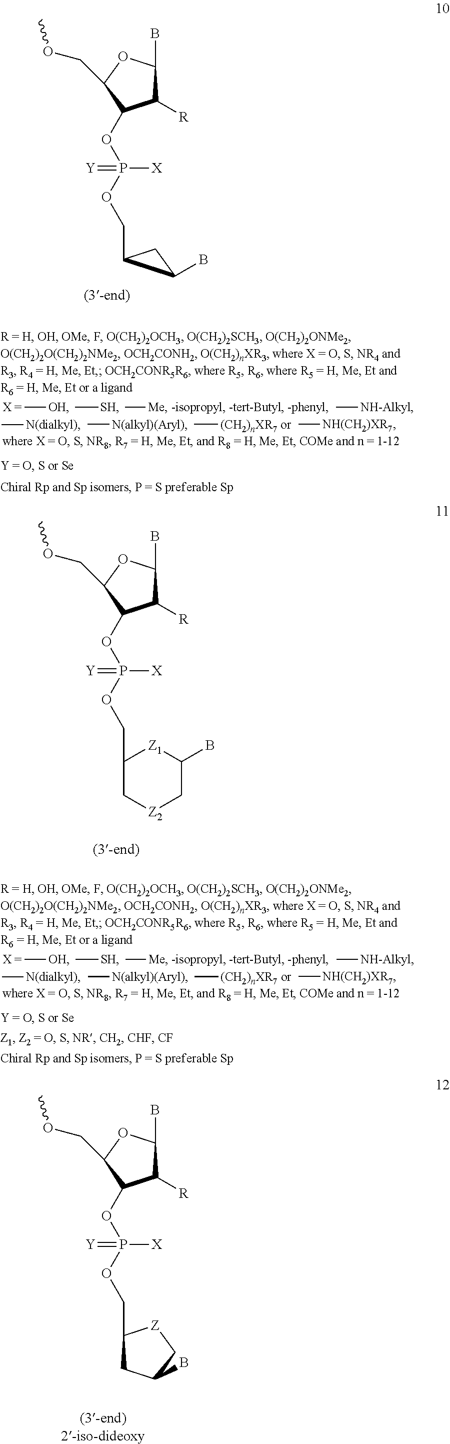

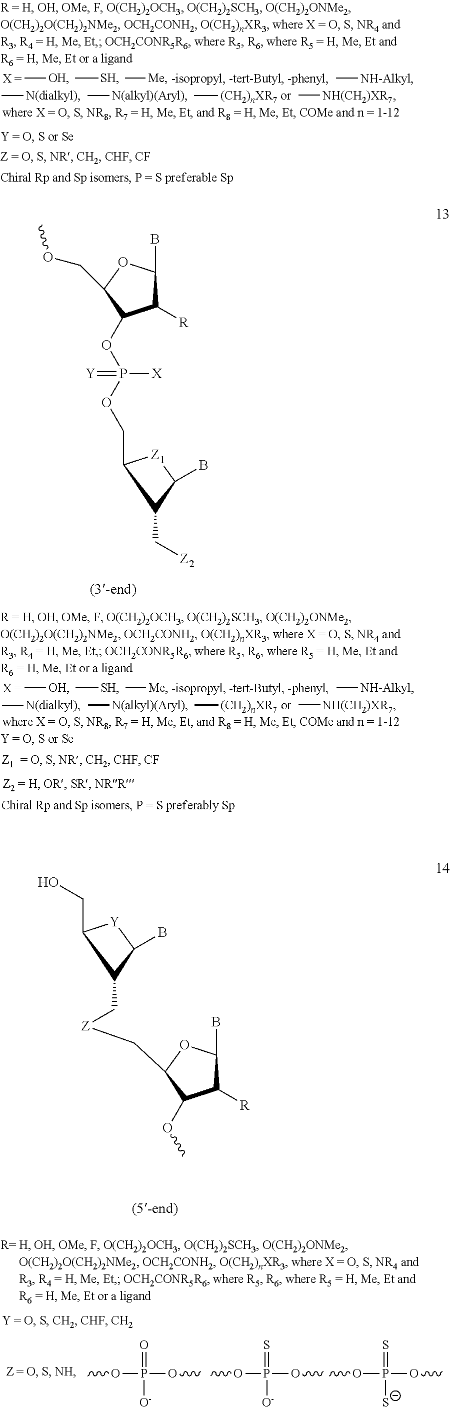

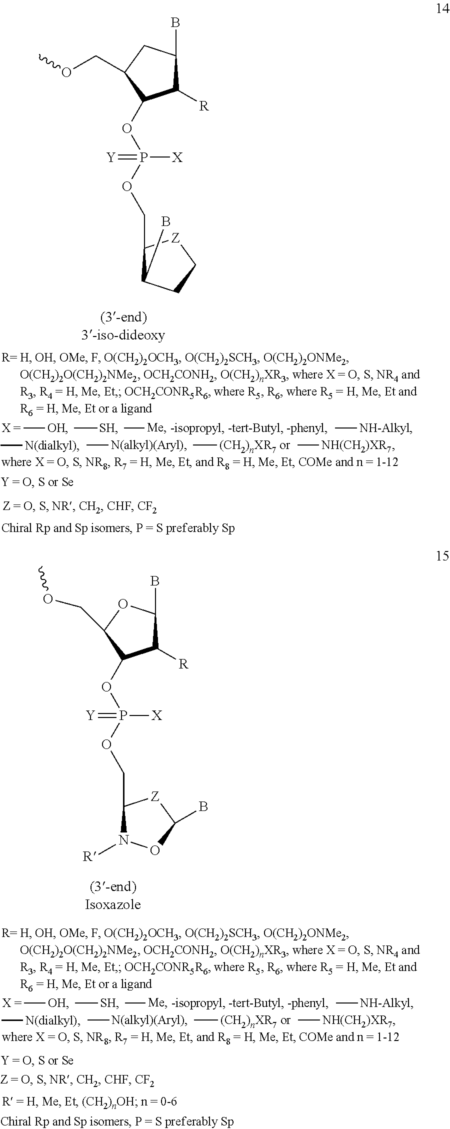

L-Nucleoside Derived from 1-15

2′-5′ Linkage from 1-15

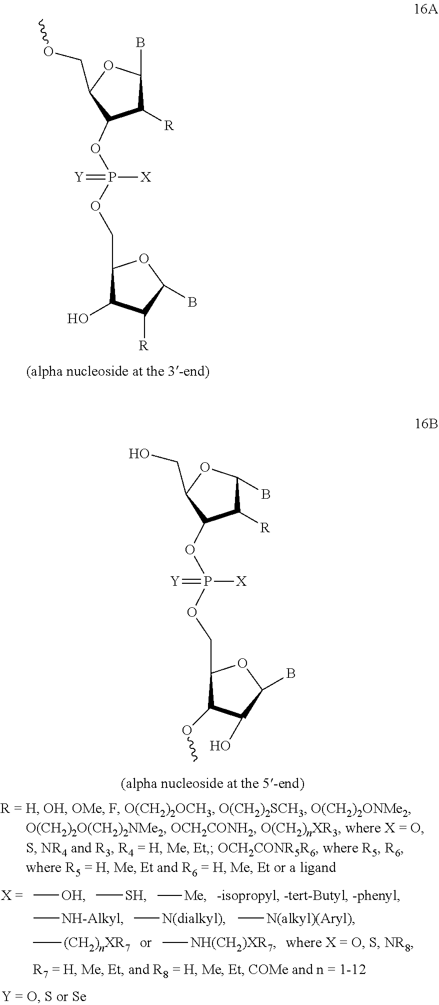

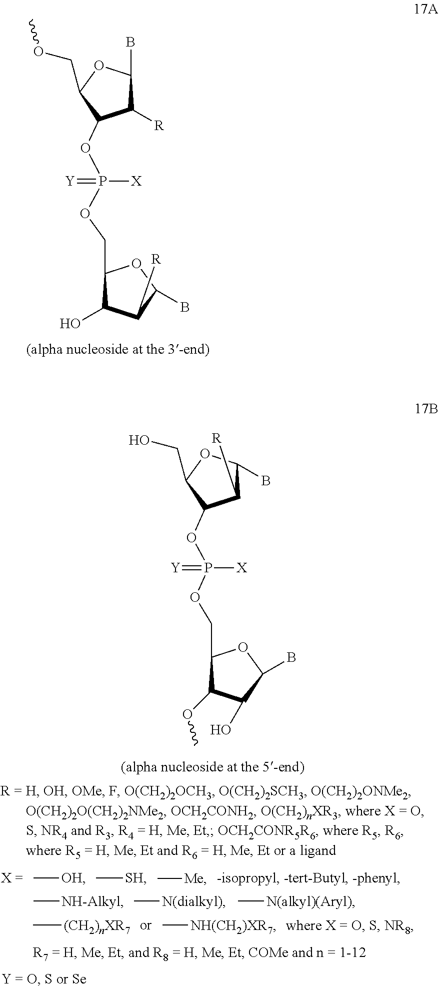

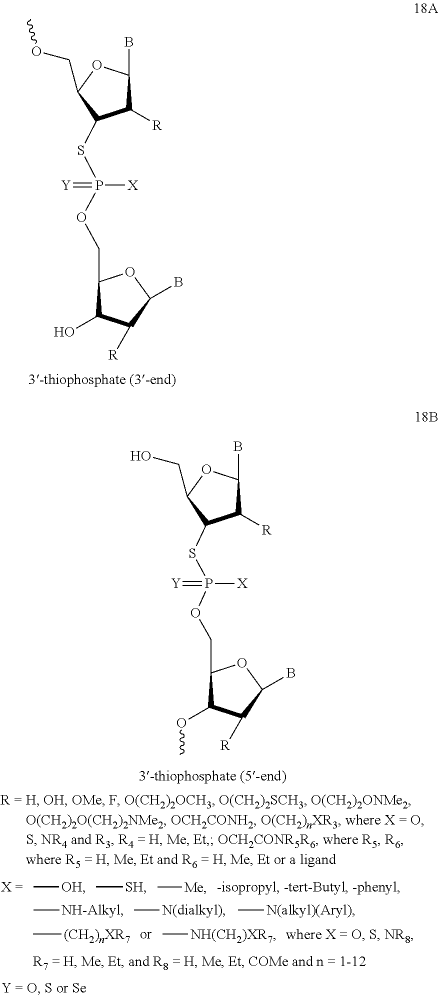

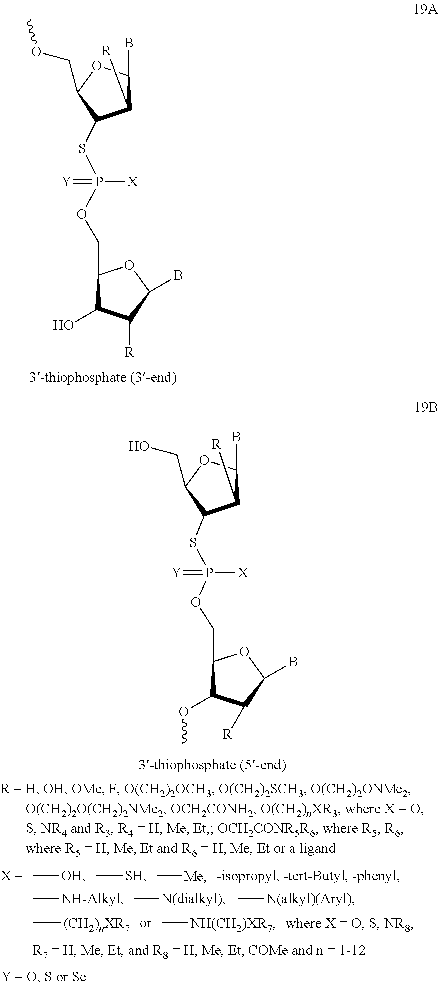

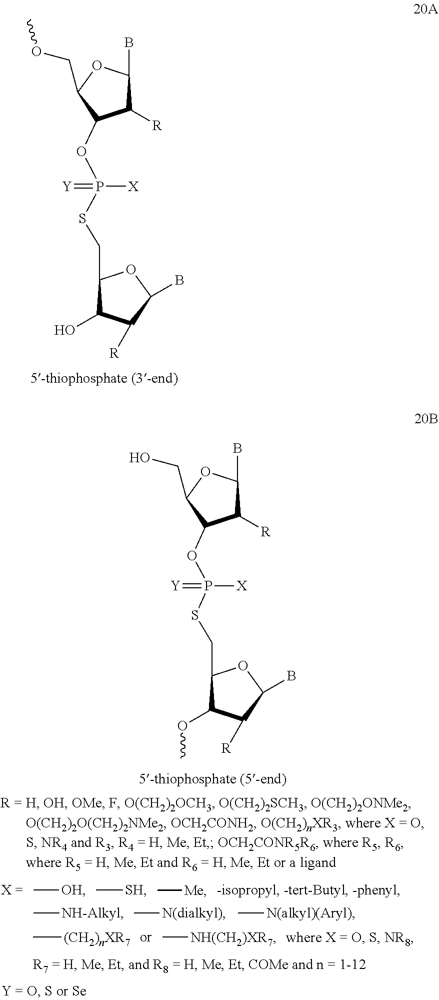

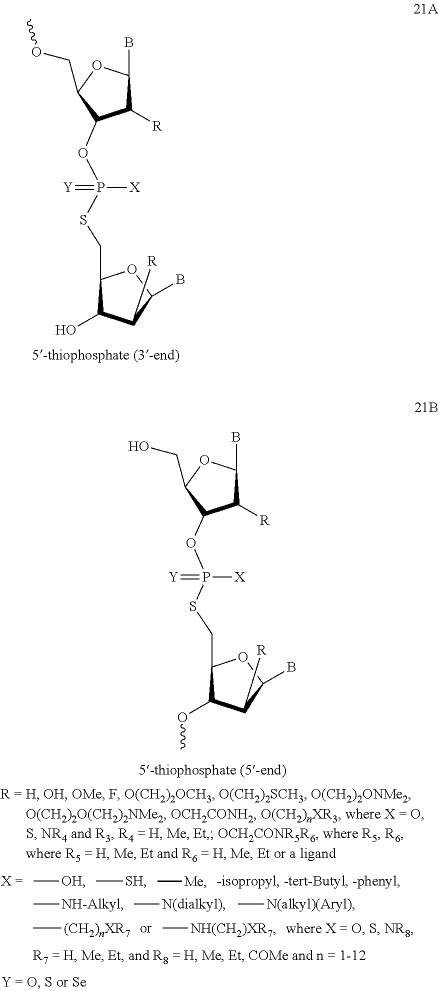

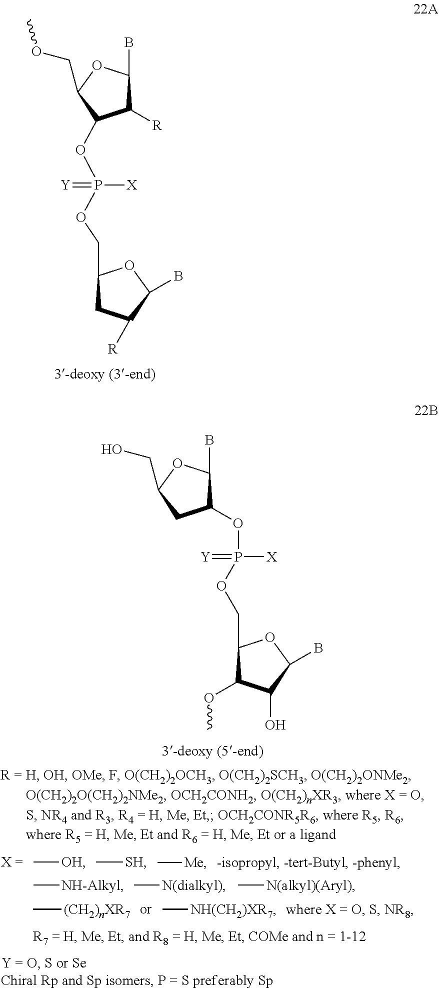

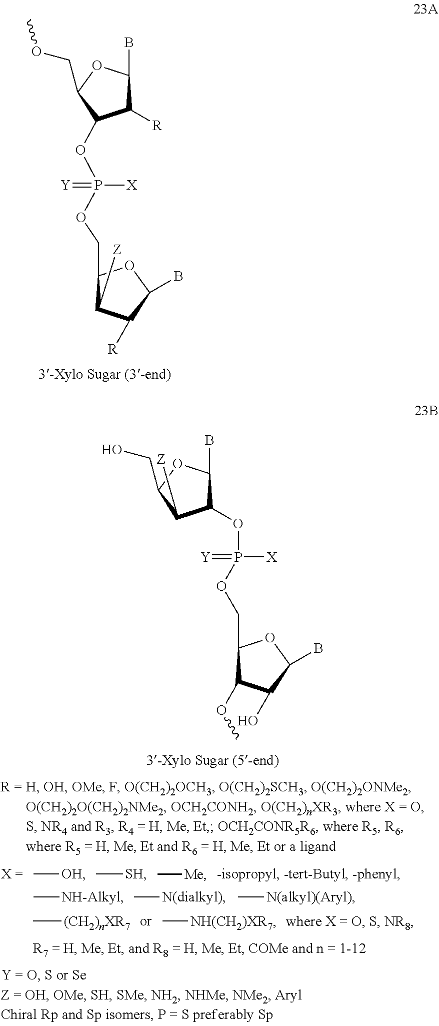

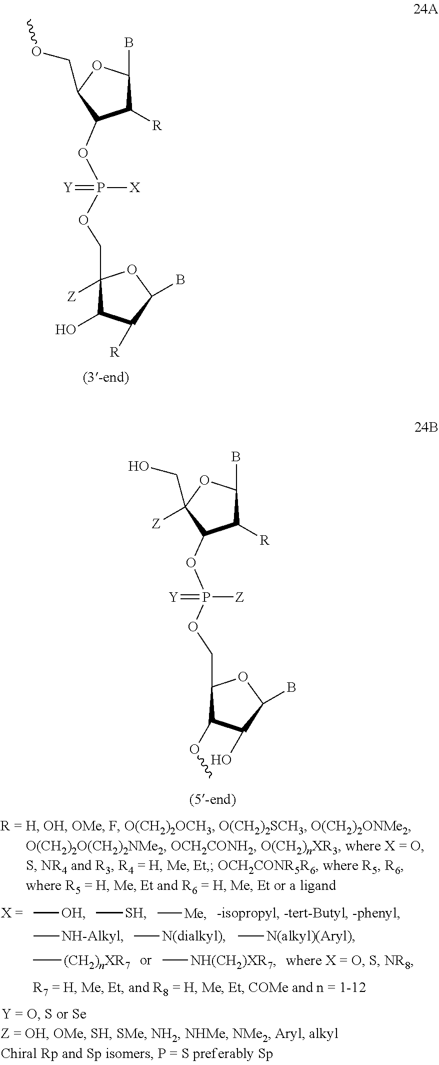

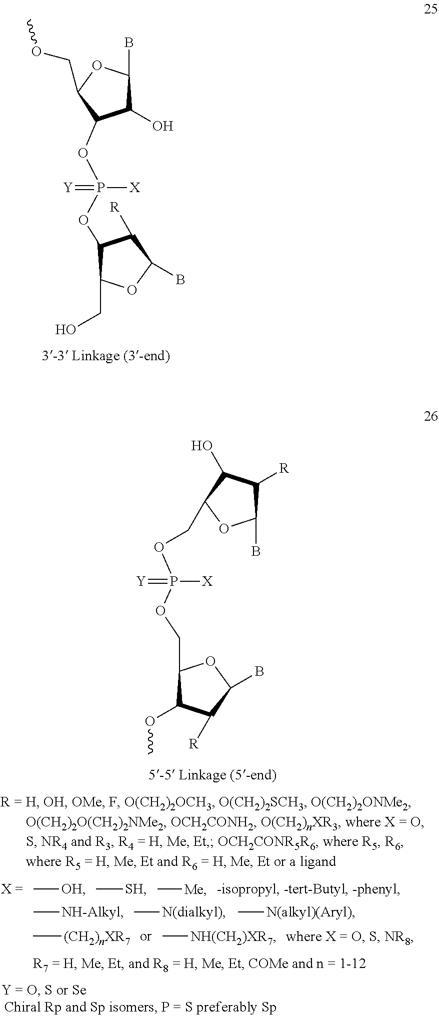

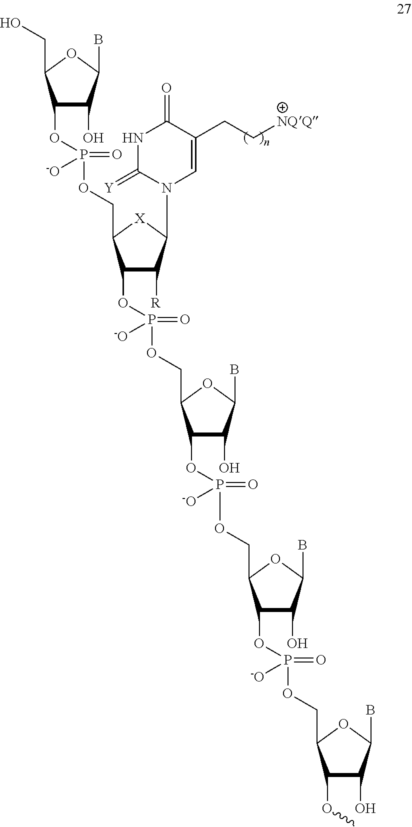

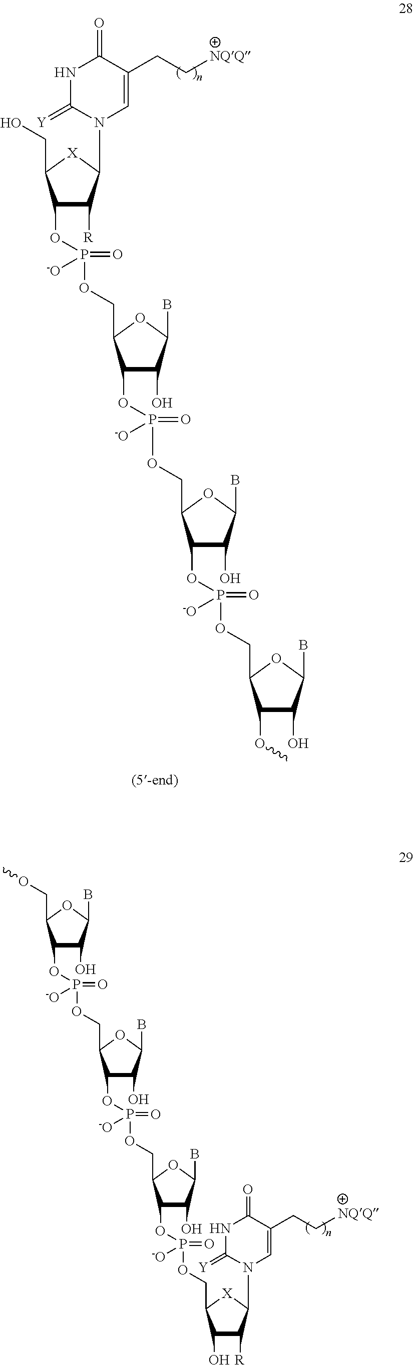

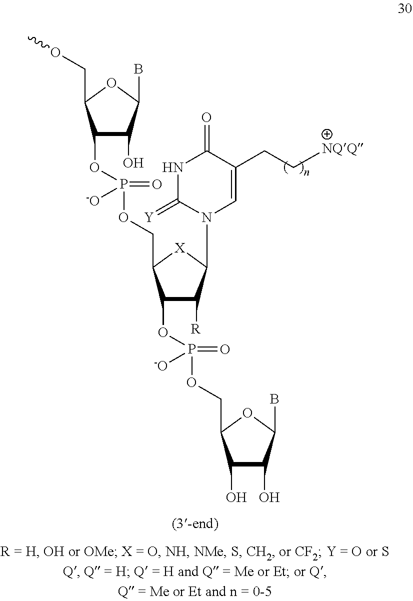

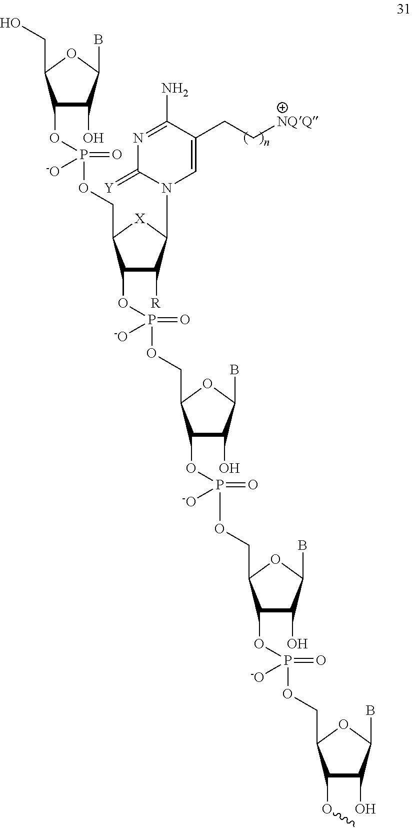

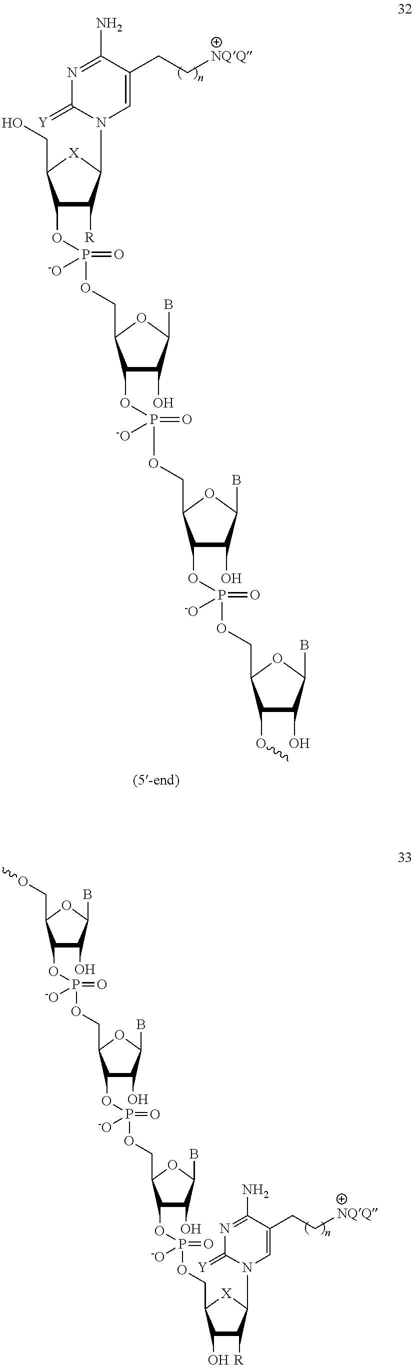

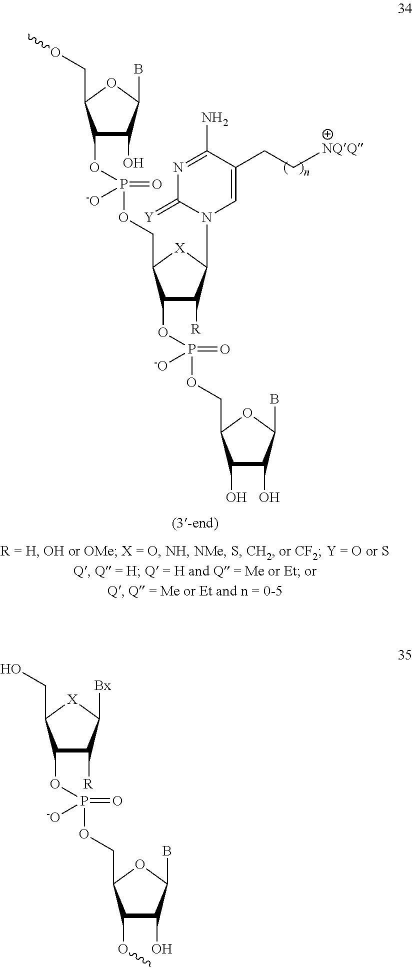

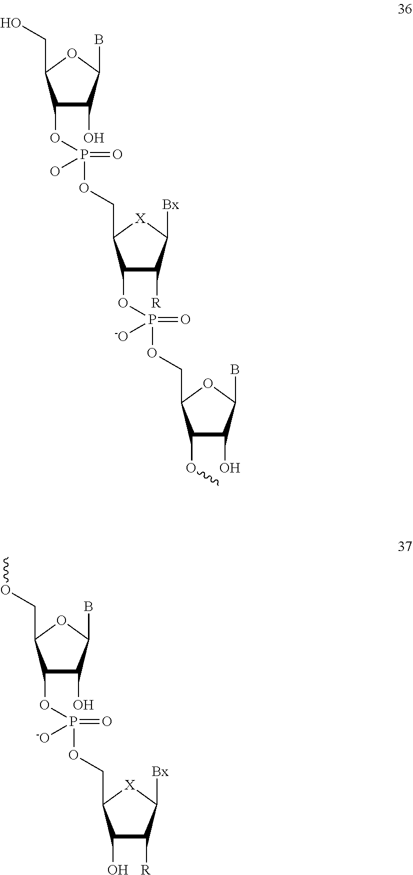

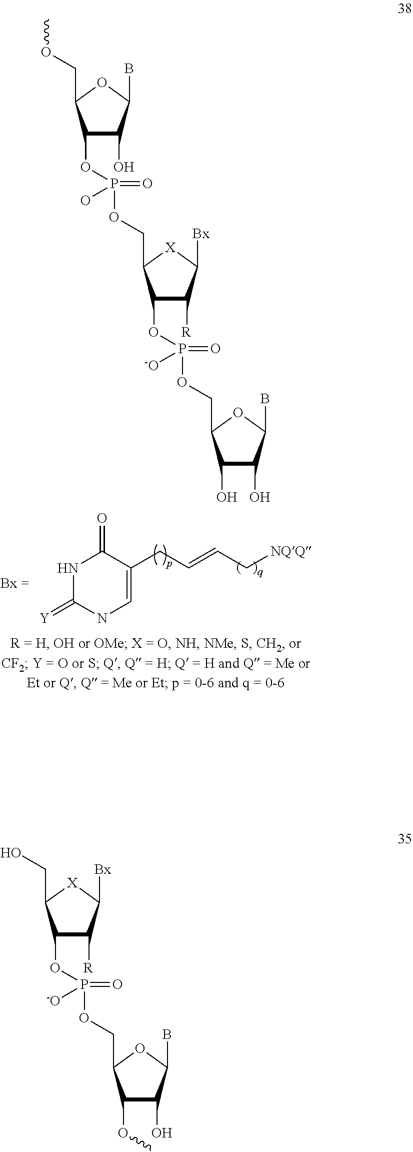

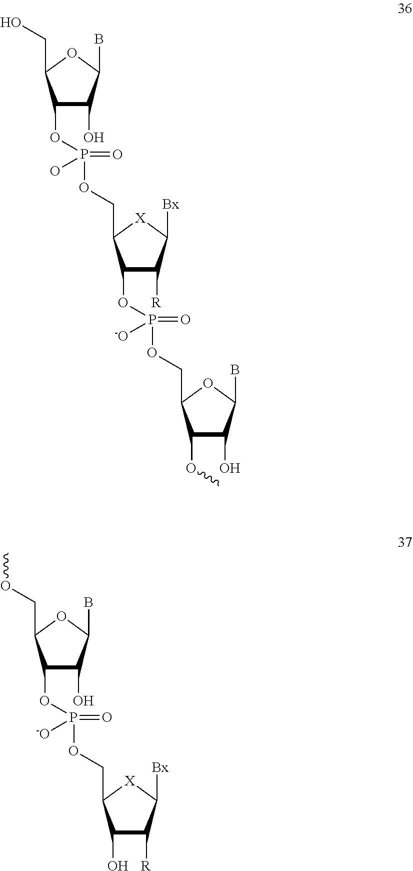

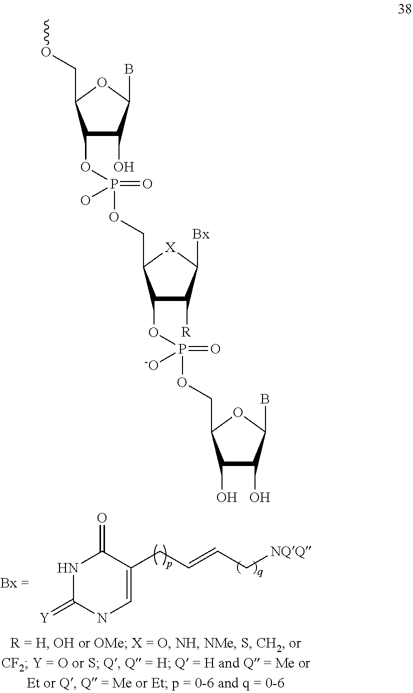

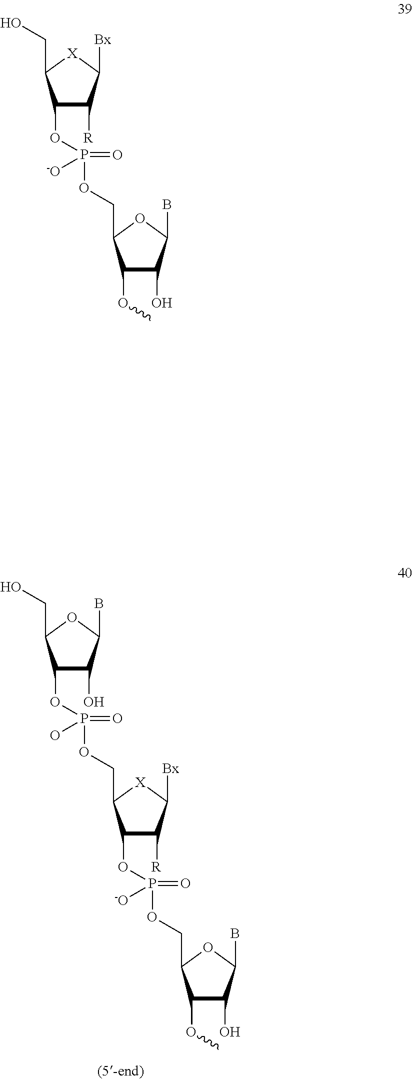

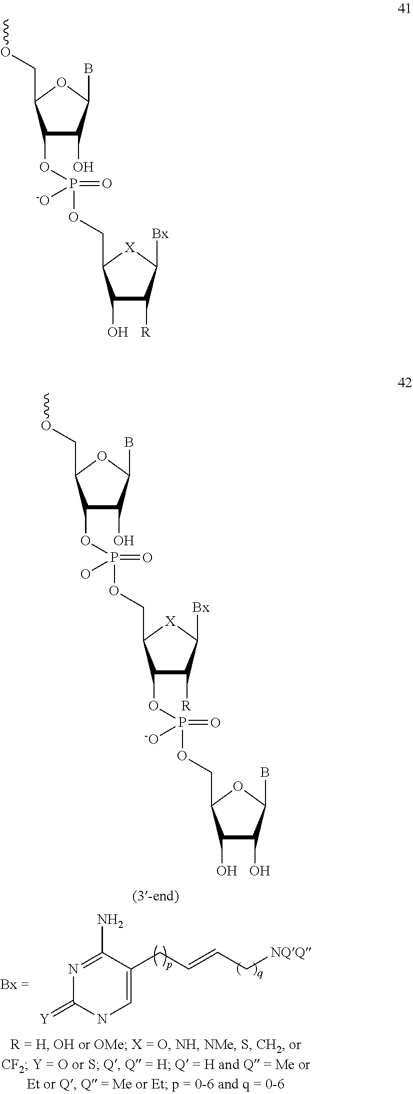

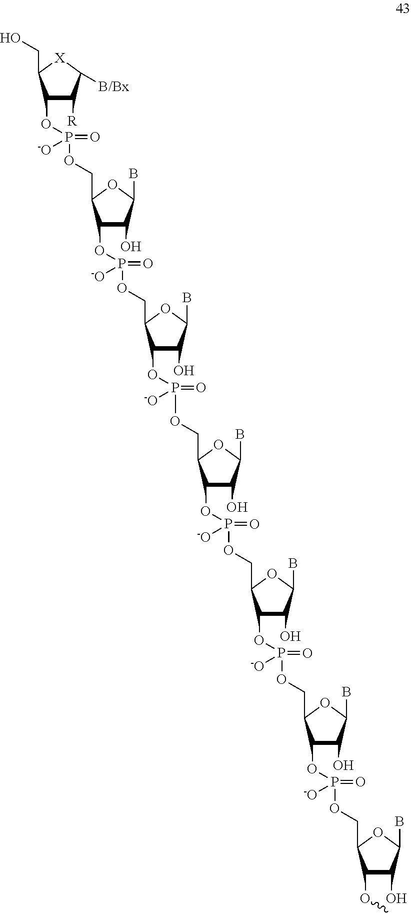

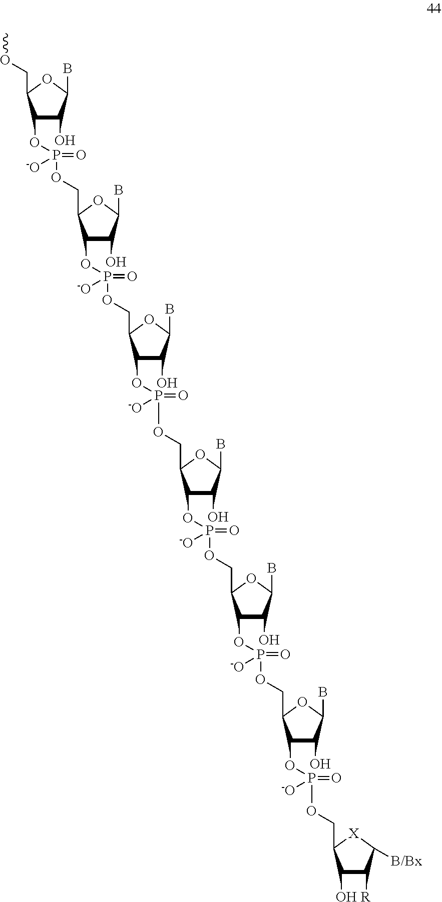

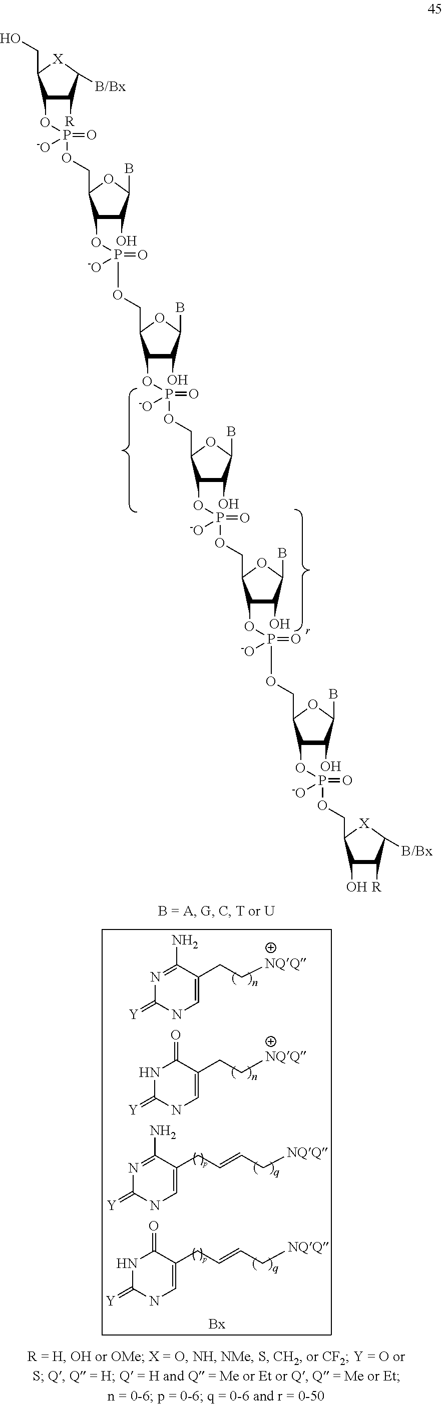

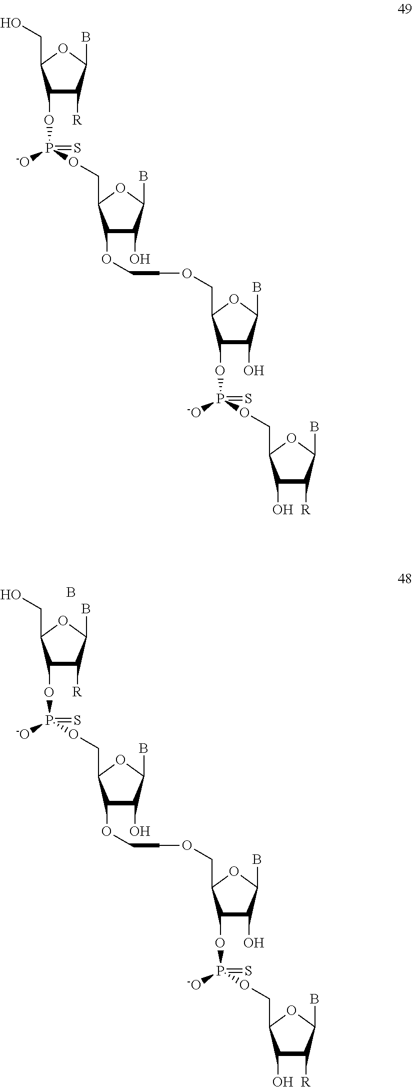

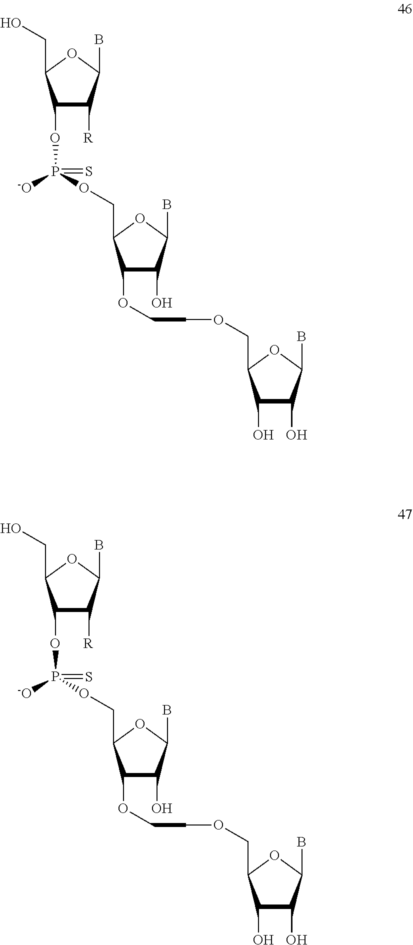

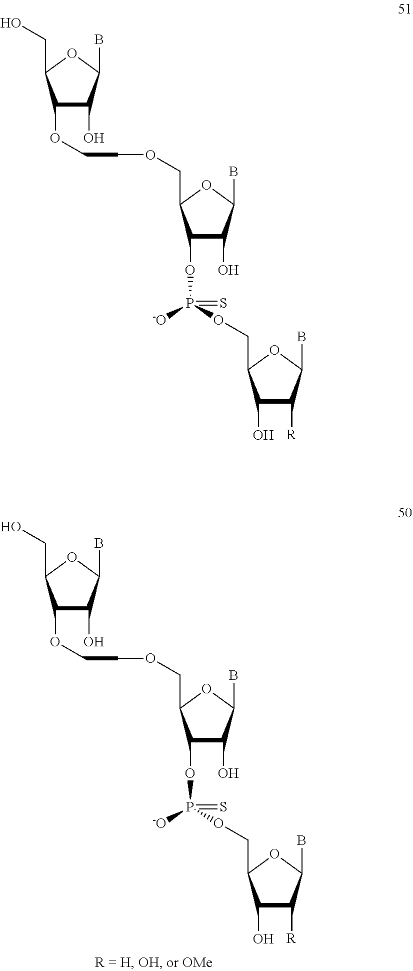

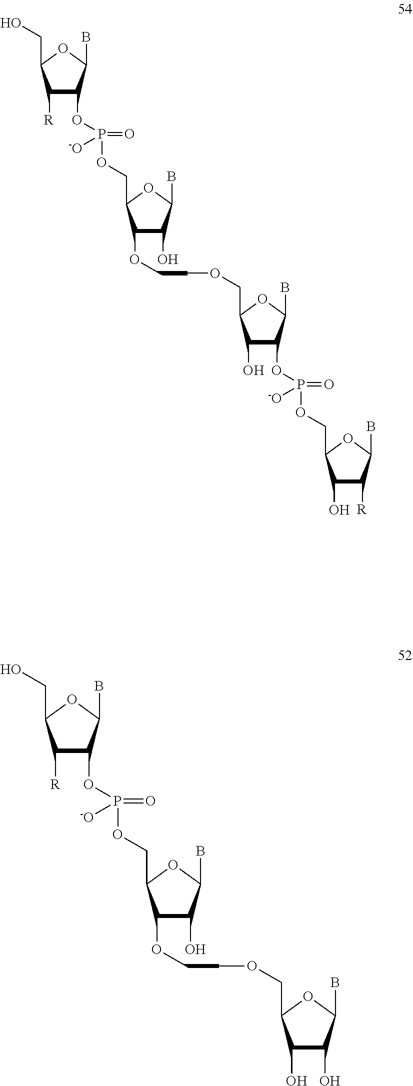

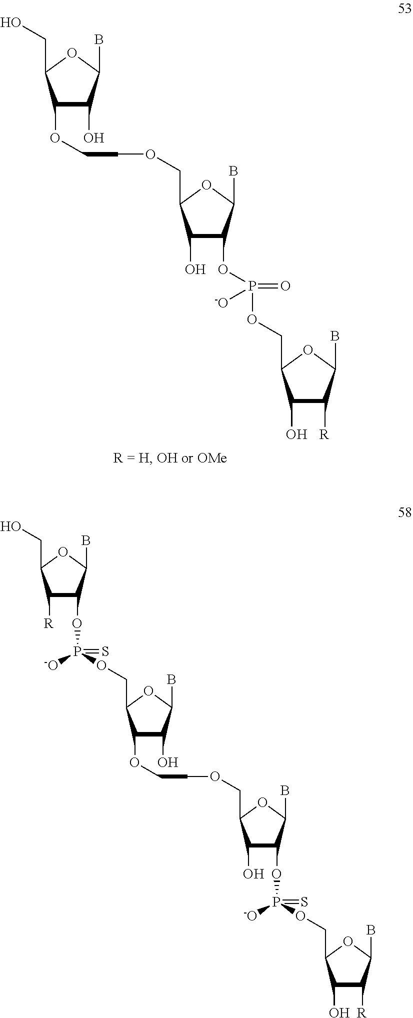

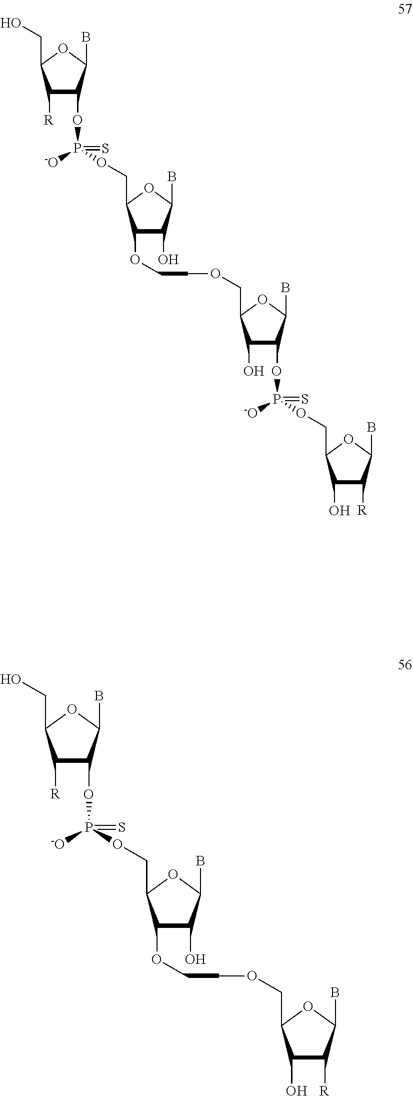

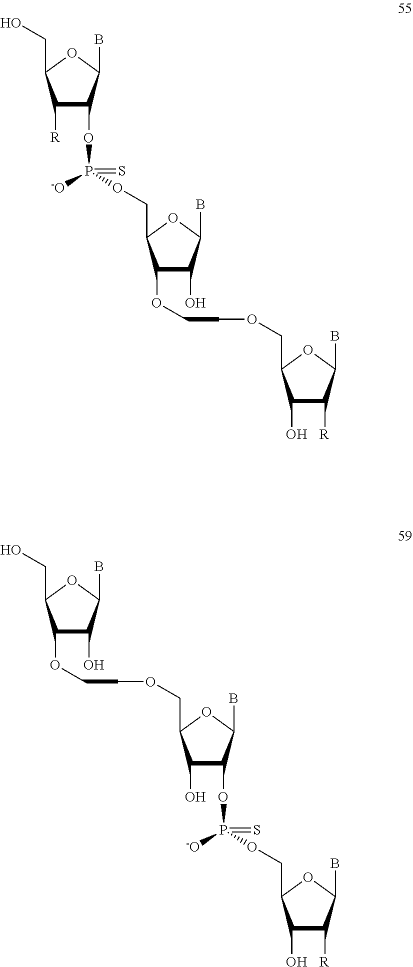

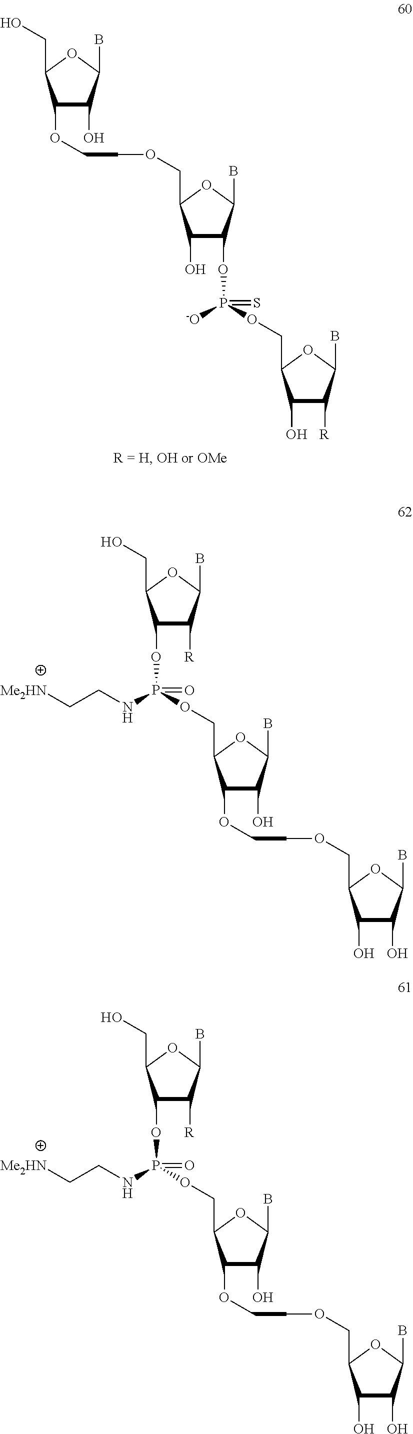

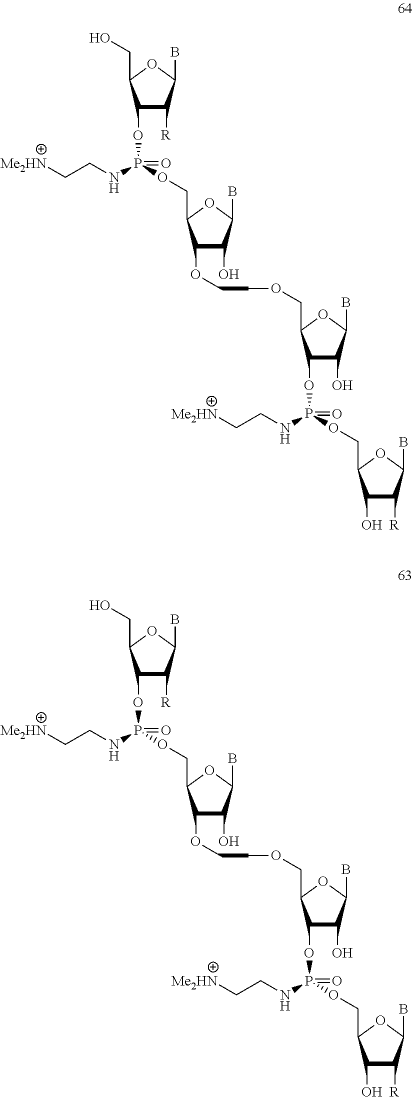

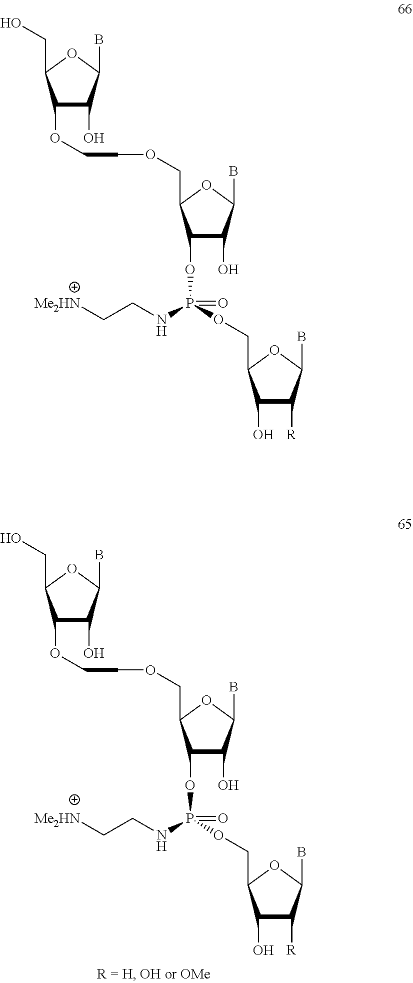

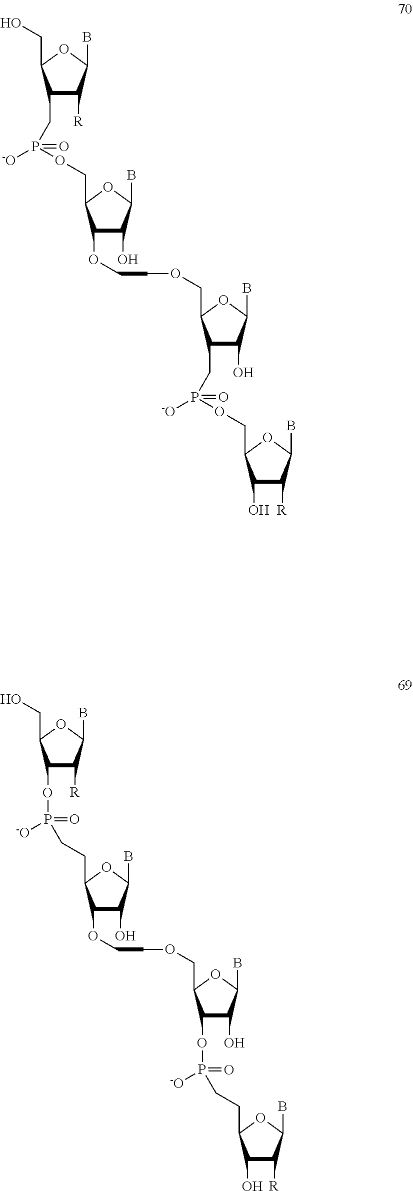

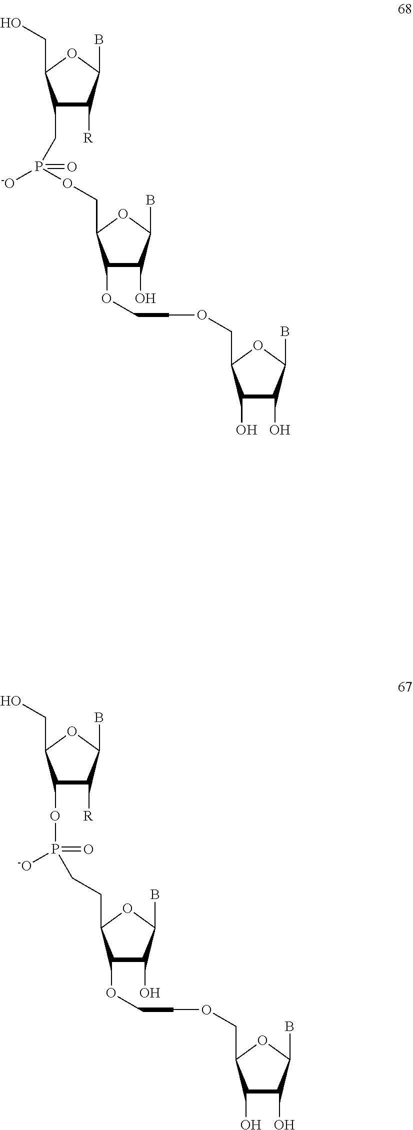

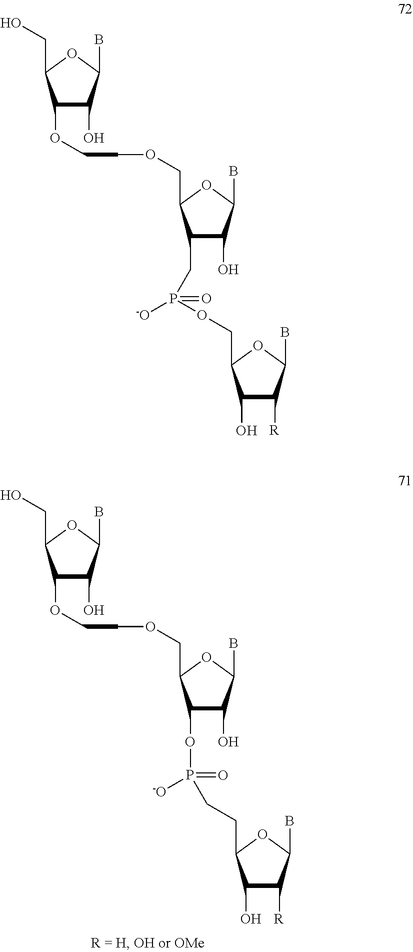

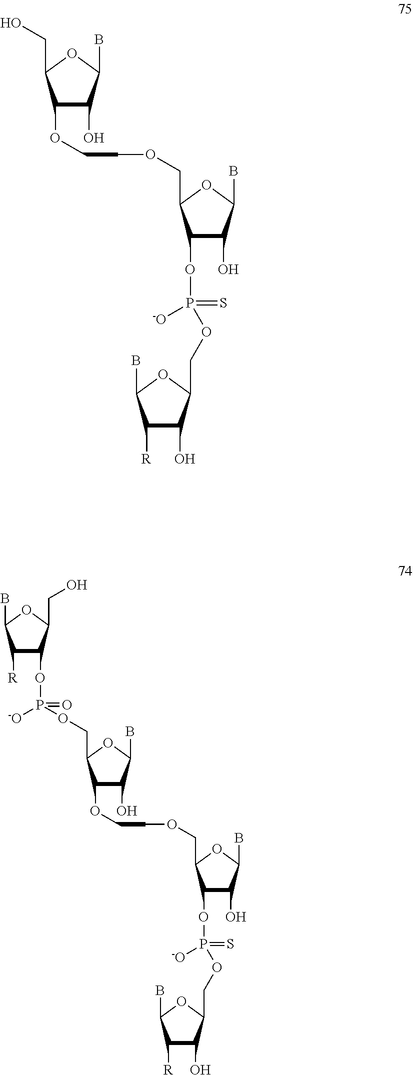

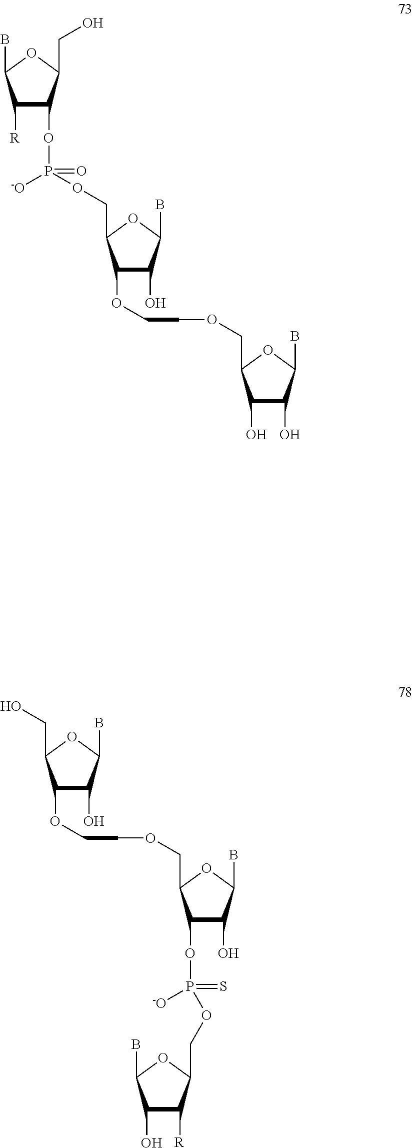

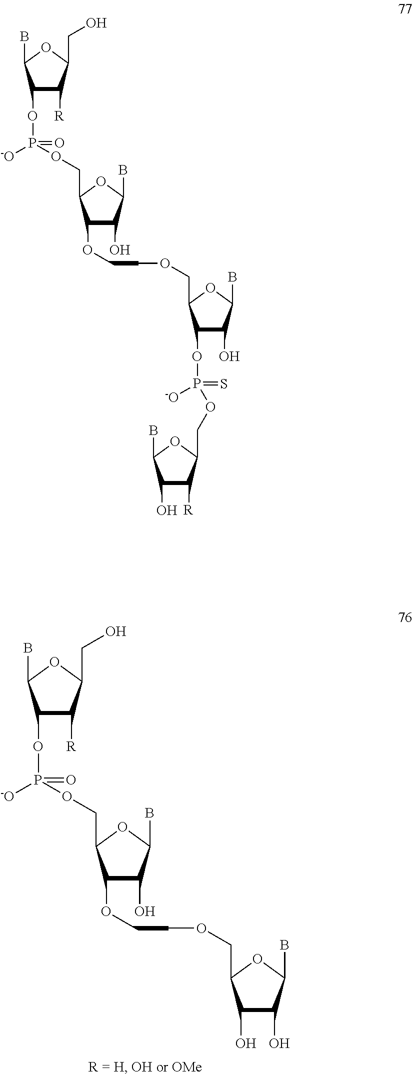

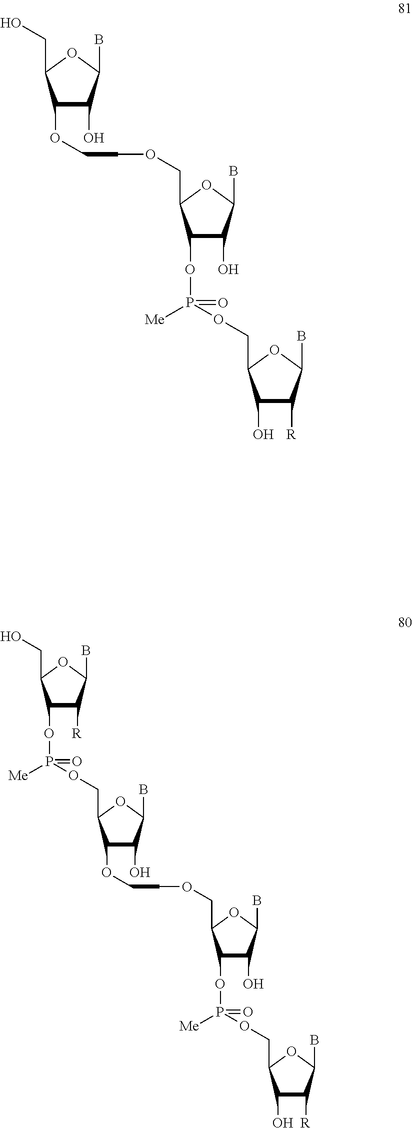

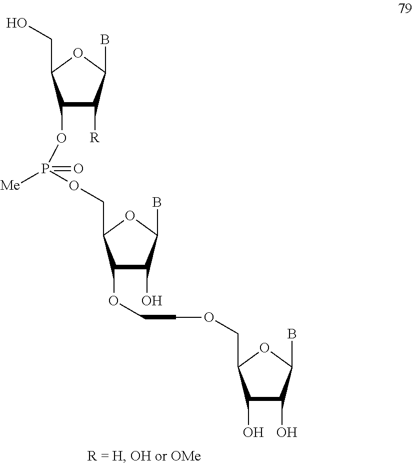

For structures 27-81, R can also have the values for structures 1A-26.

The iRNA agent can include a conjugate (also called a ligand) on the 5′ or 3′ terminus of the sense or antisense strand. Exemplary ligands are shown below. The conjugate can be attached to the 3′ or 5′ end. Preferably, a cholesterol conjugate is on the 5′ or 3′ end of the sense strand. Preferably, a cholesterol conjugate is not on the antisense strand.

### Preferred Ligands

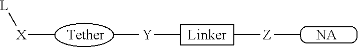

### L=

Vitamins
Peptides
Carbohydrates
Terpenes
Steroids
Lipids
Amino acids
Naproxen
Ibuprofen
Amines (mono, di, tri, tetraalkyl or aryl)
Folate
Sugar (N-Acetylgalactosamine, galactosamine, galactose, Mannose)
—(CH₂)ₙNQ₁Q₂, where n=0-40, Q₁, Q₂═H, Me or Et; Q₁═H, Q₂═H, Me, Et or aryl
—(CH₂)ₚCH═CH(CH₂)_{q}NQ₁Q₂, where p and/or q=0-40, Q₁, Q₂═H, Me or Et; Q₁═H, Q₂═H, Me, Et or aryl with E and/or Z configuration
—(CH₂)ₚCH_{≡}CH(CH₂)_{q}NQ₁Q₂, where p and/or q=0-40, Q₁, Q₂═H, Me or Et; Q₁═H, Q₂═H, Me, Et or aryl
—(CH₂)ₚCH═CH(CH₂)_{q}CH═CH(CH₂)ᵣNQ₁Q₂, where p, q and/or r=0-40, Q₁, Q₂═H, Me or Et; Q₁═H, Q₂═H, Me, Et or aryl with E and/or Z configuration
—O(CH₂)ₘ(OCH₂CH₂)ₙ—OR, where m, n=0-40 and R═H, Me, NQ₁Q₂, —C(O)NR′R″—C(S)NR′R″
—NH(CH₂)ₘ(OCH₂CH₂)ₙ—OR, where m, n=0-40 and R═H, Me, NQ₁Q₂, —C(O)NR′R″-C(S)NR′R″
—O(CH₂)ₘ(NHCH₂CH₂)ₙ—R, where m, n=0-40 and R═H, OH, Me, NQ₁Q₂, —C(O)NR′R″-C(S)NR′R″
—NH(CH₂)ₘ(NHCH₂CH₂)ₙ—R, where m, n=0-40 and R═H, OH, Me, NQ₁Q₂, —C(O)NR′R″-C(S)NR′R″
Dialkylglycerol (sn3, sn1, sn2 and racemic) with number of methylene varies from 0-40
Diacylglycerol (sn3, sn1, sn2 and racemic) with number of methylene varies from 0-40
Dialkylglycerol (sn3, sn1, sn2 and racemic) with number of methylene varies from 0-40 and the alkyl chian contains one or more double bonds with E and/or Z isomers
Diacylglycerol (sn3, sn1, sn2 and racemic) with number of methylene varies from 0-40 and the alkyl chian contains one or more double bonds with E and/or Z isomers

Double-stranded (dsRNA) directs the sequence-specific silencing of mRNA through a process known as RNA interference (RNAi). The process occurs in a wide variety of organisms, including mammals and other vertebrates.

In a typical embodiment, the subject is a mammal such as a cow, horse, mouse, rat, dog, pig, goat, or a primate. The subject can be a dairy mammal (e.g., a cow, or goat) or other farmed animal (e.g., a chicken, turkey, sheep, pig, fish, shrimp). In a much preferred embodiment, the subject is a human, e.g., a normal individual or an individual that has, is diagnosed with, or is predicted to have a disease or disorder.

### Ligand-Conjugated Monomer Subunits and Monomers for Oligonucleotide Synthesis

Definitions. The term “halo” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). Alkyl and haloalkyl groups may be optionally inserted with O, N, or S. The terms “aralkyl” refers to an alkyl moiety in which an alkyl hydrogen atom is replaced by an aryl group. Aralkyl includes groups in which more than one hydrogen atom has been replaced by an aryl group. Examples of “aralkyl” include benzyl, 9-fluorenyl, benzhydryl, and trityl groups.

The term “alkenyl” refers to a straight or branched hydrocarbon chain containing 2-8 carbon atoms and characterized in having one or more double bonds. Examples of a typical alkenyl include, but not limited to, allyl, propenyl, 2-butenyl, 3-hexenyl and 3-octenyl groups. The term “alkynyl” refers to a straight or branched hydrocarbon chain containing 2-8 carbon atoms and characterized in having one or more triple bonds. Some examples of a typical alkynyl are ethynyl, 2-propynyl, and 3-methylbutynyl, and propargyl. The sp² and sp³ carbons may optionally serve as the point of attachment of the alkenyl and alkynyl groups, respectively.

The terms “alkylamino” and “dialkylamino” refer to —NH(alkyl) and —N(alkyl)₂ radicals respectively. The term “aralkylamino” refers to a —NH(aralkyl) radical. The term “alkoxy” refers to an —O-alkyl radical, and the terms “cycloalkoxy” and “aralkoxy” refer to an —O-cycloalkyl and O-aralkyl radicals respectively. The term “siloxy” refers to a R₃SiO— radical. The term “mercapto” refers to an SH radical. The term “thioalkoxy” refers to an —S-alkyl radical.

The term “alkylene” refers to a divalent alkyl (i.e —R—), e.g —CH₂—, —CH₂CH₂—, and —CH₂CH₂CH₂—. The term “alkylenedioxo” refers to a divalent species of the structure —O—R—O—, in which R represents an alkylene.

The term “aryl” refers to an aromatic monocyclic, bicyclic, or tricyclic hydrocarbon ring system, wherein any ring atom can be substituted. Examples of aryl moieties include, but are not limited to, phenyl, naphthyl, anthracenyl, and pyrenyl.

The term “cycloalkyl” as employed herein includes saturated cyclic, bicyclic, tricyclic, or polycyclic hydrocarbon groups having 3 to 12 carbons, wherein any ring atom can be substituted. The cycloalkyl groups herein described may also contain fused rings. Fused rings are rings that share a common carbon-carbon bond or a common carbon atom (e.g., spiro-fused rings). Examples of cycloalkyl moieties include, but are not limited to, cyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heterocyclyl groups herein described may also contain fused rings. Fused rings are rings that share a common carbon-carbon bond or a common carbon atom (e.g., spiro-fused rings). Examples of heterocyclyl include, but are not limited to tetrahydrofuranyl, tetrahydropyranyl, piperidinyl, morpholino, pyrrolinyl and pyrrolidinyl.

The term “cycloalkenyl” as employed herein includes partially unsaturated, nonaromatic, cyclic, bicyclic, tricyclic, or polycyclic hydrocarbon groups having 5 to 12 carbons, preferably 5 to 8 carbons, wherein any ring atom can be substituted. The cycloalkenyl groups herein described may also contain fused rings. Fused rings are rings that share a common carbon-carbon bond or a common carbon atom (e.g., spiro-fused rings). Examples of cycloalkenyl moieties include, but are not limited to cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heterocycloalkenyl groups herein described may also contain fused rings. Fused rings are rings that share a common carbon-carbon bond or a common carbon atom (e.g., spiro-fused rings). Examples of heterocycloalkenyl include but are not limited to tetrahydropyridyl and dihydropyran.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom can be substituted. The heteroaryl groups herein described may also contain fused rings that share a common carbon-carbon bond.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The terms “adeninyl, cytosinyl, guaninyl, thyminyl, and uracilyl” and the like refer to radicals of adenine, cytosine, guanine, thymine, and uracil.

A “protected” moiety refers to a reactive functional group, e.g., a hydroxyl group or an amino group, or a class of molecules, e.g., sugars, having one or more functional groups, in which the reactivity of the functional group is temporarily blocked by the presence of an attached protecting group. Protecting groups useful for the monomers and methods described herein can be found, e.g., in Greene, T. W., Protective Groups in Organic Synthesis (John Wiley and Sons: New York), 1981, which is hereby incorporated by reference.

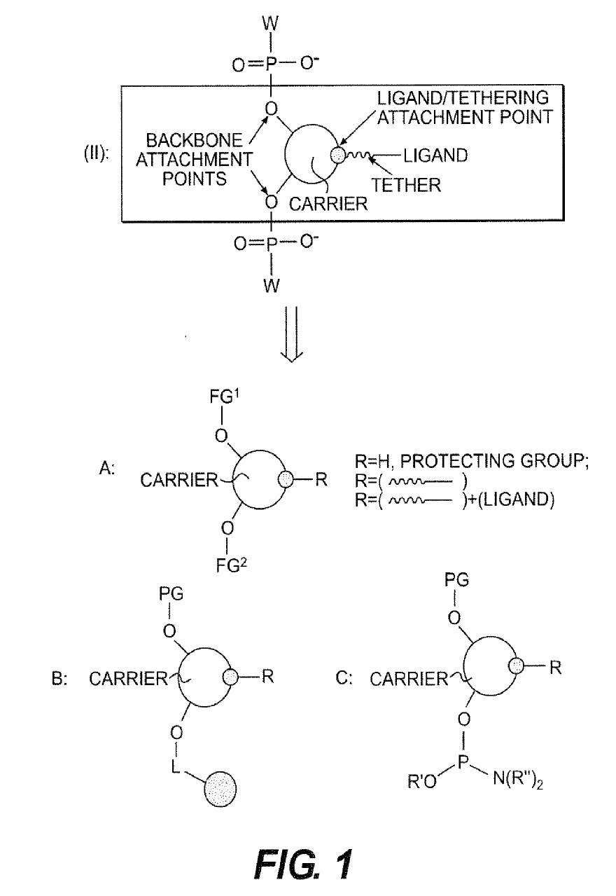

General. An RNA agent, e.g., an iRNA agent, containing a preferred, but nonlimiting ligand-conjugated monomer subunit is presented as formula (II) below and in the scheme in FIG. 1. The carrier (also referred to in some embodiments as a “linker”) can be a cyclic or acyclic moiety and includes two “backbone attachment points” (e.g., hydroxyl groups) and a ligand. The ligand can be directly attached (e.g., conjugated) to the carrier or indirectly attached (e.g., conjugated) to the carrier by an intervening tether (e.g., an acyclic chain of one or more atoms; or a nucleobase, e.g., a naturally occurring nucleobase optionally having one or more chemical modifications, e.g., an unusual base; or a universal base). The carrier therefore also includes a “ligand or tethering attachment point” for the ligand and tether/tethered ligand, respectively.

The ligand-conjugated monomer subunit may be the 5′ or 3′ terminal subunit of the RNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in a RNA molecule, e.g., an iRNA agent. Preferred positions for inclusion of a tethered ligand-conjugated monomer subunits, e.g., one in which a lipophilic moiety, e.g., cholesterol, is tethered to the carrier are at the 3′ terminus, the 5′ terminus, or an internal position of the sense strand.

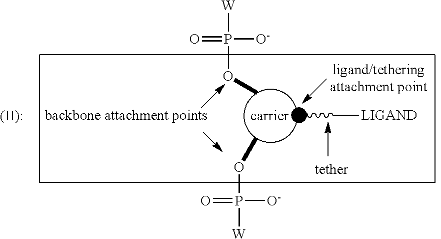

The modified RNA molecule of formula (II) can be obtained using oligonucleotide synthetic methods known in the art. In a preferred embodiment, the modified RNA molecule of formula (II) can be prepared by incorporating one or more of the corresponding monomer compounds (see, e.g., A, B, and C below and in the scheme in FIG. 1) into a growing sense or antisense strand, utilizing, e.g., phosphoramidite or H-phosphonate coupling strategies.

The monomers, e.g., a ligand-conjugated monomer, generally include two differently functionalized hydroxyl groups (OFG¹ and OFG²), which are linked to the carrier molecule (see A below and in FIG. 1), and a ligand/tethering attachment point. As used herein, the term “functionalized hydroxyl group” means that the hydroxyl proton has been replaced by another substituent. As shown in representative structures B and C below and in FIG. 1, one hydroxyl group (OFG¹) on the carrier is functionalized with a protecting group (PG). The other hydroxyl group (OFG²) can be functionalized with either (1) a liquid or solid phase synthesis support reagent (solid circle) directly or indirectly through a linker, L, as in B, or (2) a phosphorus-containing moiety, e.g., a phosphoramidite as in C. The tethering attachment point may be connected to a hydrogen atom, a suitable protecting group, a tether, or a tethered ligand at the time that the monomer is incorporated into the growing sense or antisense strand (see variable “R” in A below). Thus, the tethered ligand can be, but need not be attached to the monomer at the time that the monomer is incorporated into the growing strand. In certain embodiments, the tether, the ligand or the tethered ligand may be linked to a “precursor” ligand-conjugated monomer subunit after a “precursor” ligand-conjugated monomer subunit has been incorporated into the strand. The wavy line used below (and elsewhere herein) refers to a connection, and can represent a direct bond between the moiety and the attachment point or a tethering molecule, which is interposed between the moiety and the attachment point. Directly tethered means the moiety is bound directly to the attachment point. Indirectly tethered means that there is a tether molecule interposed between the attachment point and the moiety.

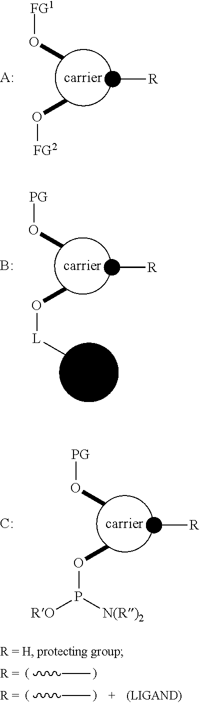

The (OFG¹) protecting group may be selected as desired, e.g., from T. W. Greene and P. G. M. Wuts, Protective Groups in Organic Synthesis, 2d. Ed., John Wiley and Sons (1991). The protecting group is preferably stable under amidite synthesis conditions, storage conditions, and oligonucleotide synthesis conditions. Hydroxyl groups, —OH, are nucleophilic groups (i.e., Lewis bases), which react through the oxygen with electrophiles (i.e., Lewis acids). Hydroxyl groups in which the hydrogen has been replaced with a protecting group, e.g., a triarylmethyl group or a trialkylsilyl group, are essentially unreactive as nucleophiles in displacement reactions. Thus, the protected hydroxyl group is useful in preventing e.g., homocoupling of compounds exemplified by structure C during oligonucleotide synthesis. In some embodiments, a preferred protecting group is the dimethoxytrityl group. In other embodiments, a preferred protecting group is a silicon-based protecting group having the formula below:

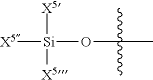

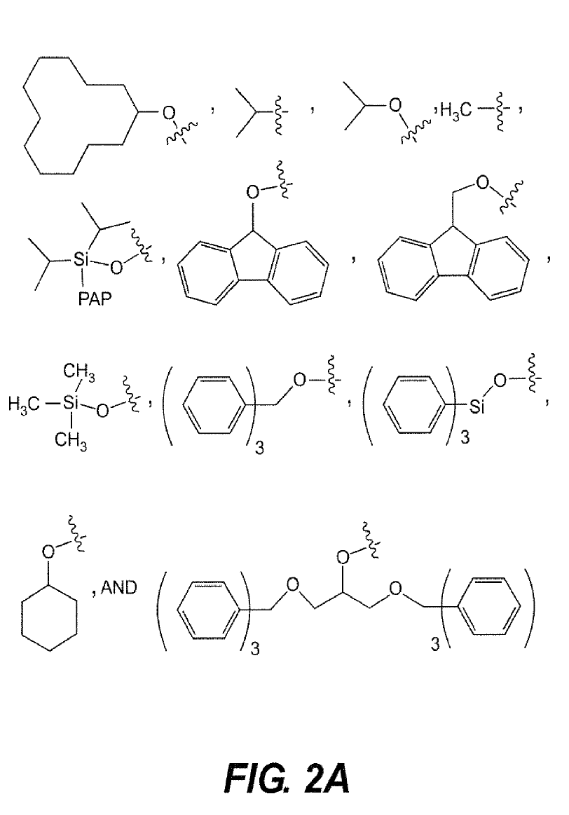

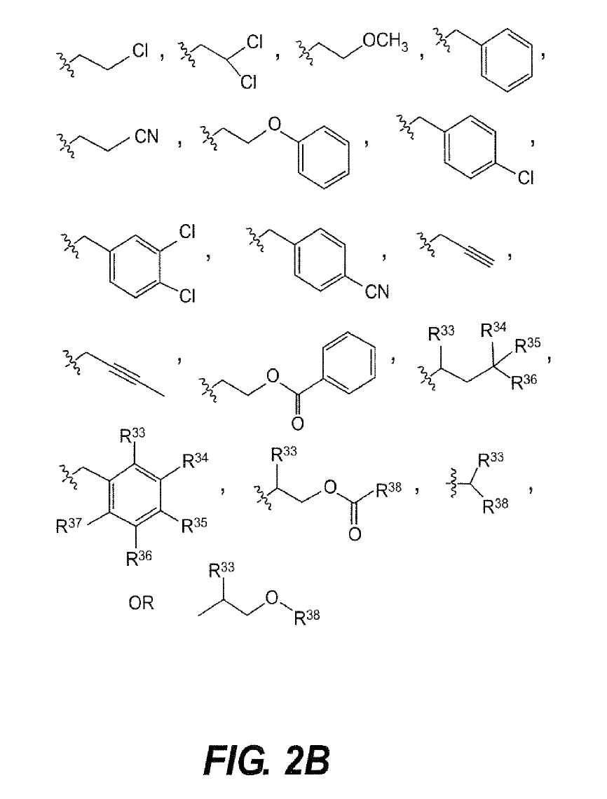

X⁵′, X⁵″, and X⁵′″ can be selected from substituted or unsubstituted alkyl, cycloalkyl, aryl, araklyl, heteroaryl, alkoxy, cycloalkoxy, aralkoxy, aryloxy, heteroaryloxy, or siloxy (i.e., R₃SiO—, the three “R” groups can be any combination of the above listed groups). X⁵′, X⁵″, and X⁵′″ may all be the same or different; also contemplated is a combination in which two of X⁵′, X⁵″, and X⁵′″ are identical and the third is different. In certain embodiments X⁵′, X⁵″, and X⁵′″ include at least one alkoxy or siloxy groups and may be any one of the groups listed in FIG. 2A, a preferred combination includes X⁵′, X⁵″=trimethylsiloxy and X⁵′″=1,3-(tri-2-propoxy or cyclododecyloxy.

Other preferred combinations of X⁵′, X⁵″, and X⁵′″ include those that result in OFG¹ groups that meet the deprotection and stability criteria delineated below. The group is preferably stable under amidite synthesis conditions, storage conditions, and oligonucleotide synthesis conditions. Rapid removal, i.e., less than one minute, of the silyl group from e.g., a support-bound oligonucleotide is desirable because it can reduce synthesis times and thereby reduce exposure time of the growing oligonucleotide chain to the reagents. Oligonucleotide synthesis can be improved if the silyl protecting group is visible during deprotection, e.g., from the addition of a chromophore silyl substituent.

Selection of silyl protecting groups can be complicated by the competing demands of the essential characteristics of stability and facile removal, and the need to balance these competitive goals. Most substituents that increase stability can also increase the reaction time required for removal of the silyl group, potentially increasing the level of difficulty in removal of the group.

The addition of alkoxy and siloxy substituents to OFG¹ silicon-containing protecting groups increases the susceptibility of the protecting groups to fluoride cleavage of the silylether bonds. Increasing the steric bulk of the substituents preserves stability while not decreasing fluoride lability to an equal extent. An appropriate balance of substituents on the silyl group makes a silyl ether a viable nucleoside protecting group.

Candidate OFG¹ silicon-containing protecting groups may be tested by exposing a tetrahydrofuran solution of a preferred carrier bearing the candidate OFG¹ group to five molar equivalents of tetrahydrofuran at room temperature. The reaction time may be determined by monitoring the disappearance of the starting material by thin layer chromatography.

When the OFG² in B includes a linker, e.g., a relatively long organic linker, connected to a soluble or insoluble support reagent, solution or solid phase synthesis techniques can be employed to build up a chain of natural and/or modified ribonucleotides once OFG¹ is deprotected and free to react as a nucleophile with another nucleoside or monomer containing an electrophilic group (e.g., an amidite group). Alternatively, a natural or modified ribonucleotide or oligoribonucleotide chain can be coupled to monomer C via an amidite group or H-phosphonate group at OFG². Subsequent to this operation, OFG¹ can be deblocked, and the restored nucleophilic hydroxyl group can react with another nucleoside or monomer containing an electrophilic group. R′ can be substituted or unsubstituted alkyl or alkenyl. In preferred embodiments, R′ is methyl, allyl or 2-cyanoethyl. R″ may a C₁-C₁₀ alkyl group, preferably it is a branched group containing three or more carbons, e.g., isopropyl.

OFG² in B can be hydroxyl functionalized with a linker, which in turn contains a liquid or solid phase synthesis support reagent at the other linker terminus. The support reagent can be any support medium that can support the monomers described herein. The monomer can be attached to an insoluble support via a linker, L, which allows the monomer (and the growing chain) to be solubilized in the solvent in which the support is placed. The solubilized, yet immobilized, monomer can react with reagents in the surrounding solvent; unreacted reagents and soluble by-products can be readily washed away from the solid support to which the monomer or monomer-derived products is attached. Alternatively, the monomer can be attached to a soluble support moiety, e.g., polyethylene glycol (PEG) and liquid phase synthesis techniques can be used to build up the chain. Linker and support medium selection is within skill of the art. Generally the linker may be —C(O)(CH₂)_{q}C(O)—, or —C(O)(CH₂)_{q}S—, in which q can be 0, 1, 2, 3, or 4; preferably, it is oxalyl, succinyl or thioglycolyl. Standard control pore glass solid phase synthesis supports can not be used in conjunction with fluoride labile 5′ silyl protecting groups because the glass is degraded by fluoride with a significant reduction in the amount of full-length product. Fluoride-stable polystyrene based supports or PEG are preferred.

The ligand/tethering attachment point can be any divalent, trivalent, tetravalent, pentavalent or hexavalent atom. In some embodiments, ligand/tethering attachment point can be a carbon, oxygen, nitrogen or sulfur atom. For example, a ligand/tethering attachment point precursor functional group can have a nucleophilic heteroatom, e.g., —SH, —NH₂, secondary amino, ONH₂, or NH₂NH₂. As another example, the ligand/tethering attachment point precursor functional group can be an olefin, e.g., —CH═CH₂, and the precursor functional group can be attached to a ligand, a tether, or tethered ligand using, e.g., transition metal catalyzed carbon-carbon (for example olefin metathesis) processes or cycloadditions (e.g., Diels-Alder). As a further example, the ligand/tethering attachment point precursor functional group can be an electrophilic moiety, e.g., an aldehyde. When the carrier is a cyclic carrier, the ligand/tethering attachment point can be an endocyclic atom (i.e., a constituent atom in the cyclic moiety, e.g., a nitrogen atom) or an exocyclic atom (i.e., an atom or group of atoms attached to a constituent atom in the cyclic moiety).

The carrier can be any organic molecule containing attachment points for OFG¹, OFG², and the ligand. In certain embodiments, carrier is a cyclic molecule and may contain heteroatoms (e.g., O, N or S). E.g., carrier molecules may include aryl (e.g., benzene, biphenyl, etc.), cycloalkyl (e.g., cyclohexane, cis or trans decalin, etc.), cycloalkenyl (e.g., cyclohexenyl), or heterocyclyl (tetrahydropyran, piperazine, pyrrolidine, etc.). In other embodiments, the carrier can be an acyclic moiety, e.g., based on serinol. Any of the above cyclic systems may include substituents in addition to OFG¹, OFG², and the ligand.

Sugar-Based Monomers. In some embodiments, the carrier molecule is an oxygen containing heterocycle. Preferably the carrier is a ribose sugar as shown in structure LCM-I. In this embodiment, the monomer, e.g., a ligand-conjugated monomer is a nucleoside.

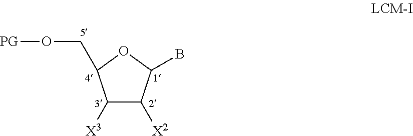

“B” represents a nucleobase, e.g., a naturally occurring nucleobase optionally having one or more chemical modifications, e.g., and unusual base; or a universal base.

As used herein, an “unusual” nucleobase can include any one of the following:
2-methyladeninyl,
N6-methyladeninyl,
2-methylthio-N6-methyladeninyl,
N6-isopentenyladeninyl,
2-methylthio-N6-isopentenyladeninyl,
N6-(cis-hydroxyisopentenyl)adeninyl,
2-methylthio-N6-(cis-hydroxyisopentenyl) adeninyl,
N6-glycinylcarbamoyladeninyl,
N6-threonylcarbamoyladeninyl,
2-methylthio-N6-threonyl carbamoyladeninyl,
N6-methyl-N6-threonylcarbamoyladeninyl,
N6-hydroxynorvalylcarbamoyladeninyl,
2-methylthio-N6-hydroxynorvalyl carbamoyladeninyl,
N6,N6-dimethyladeninyl,
3-methylcytosinyl,
5-methylcytosinyl,
2-thiocytosinyl,
5-formylcytosinyl,

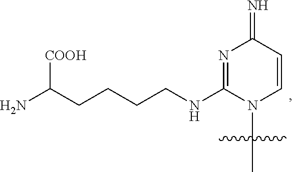

N4-methylcytosinyl,
5-hydroxymethylcytosinyl,
1-methylguaninyl,
N2-methylguaninyl,
7-methylguaninyl,
N2,N2-dimethylguaninyl,

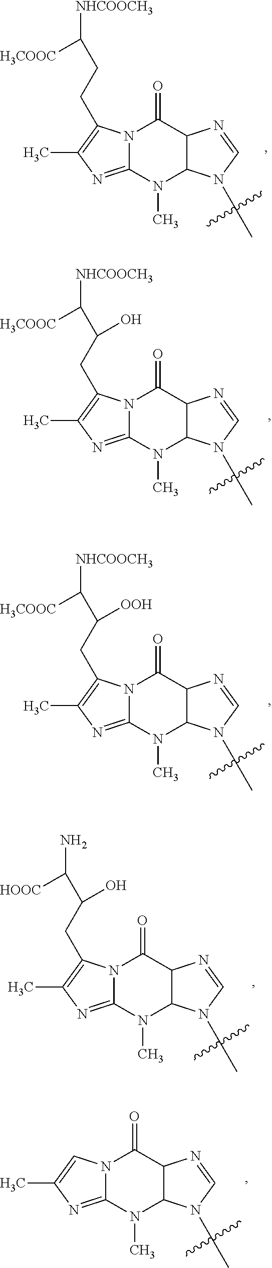

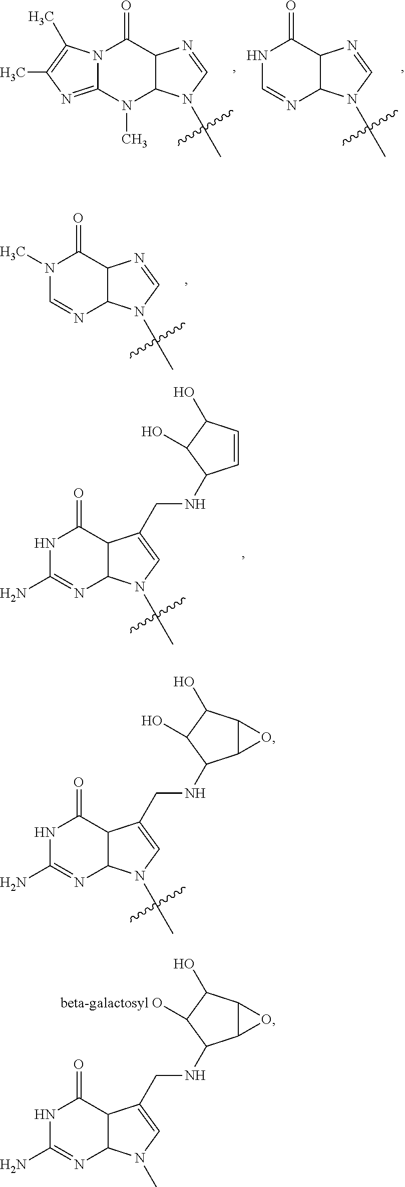

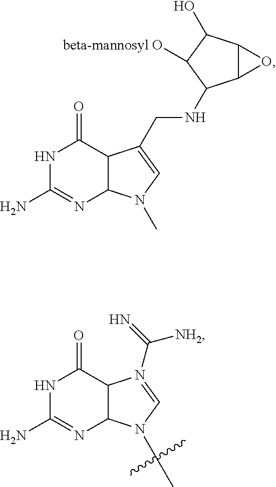

N2,7-dimethylguaninyl,
N2,N2,7-trimethylguaninyl,
1-methylguaninyl,
7-cyano-7-deazaguaninyl,
7-aminomethyl-7-deazaguaninyl,
pseudouracilyl,
dihydrouracilyl,
5-methyluracilyl,
1-methylpseudouracilyl,
2-thiouracilyl,
4-thiouracilyl,
2-thiothyminyl
5-methyl-2-thiouracilyl,
3-(3-amino-3-carboxypropyl)uracilyl,
5-hydroxyuracilyl,
5-methoxyuracilyl,
uracilyl 5-oxyacetic acid,
uracilyl 5-oxyacetic acid methyl ester,
5-(carboxyhydroxymethyl)uracilyl,
5-(carboxyhydroxymethyl)uracilyl methyl ester,
5-methoxycarbonylmethyluracilyl,
5-methoxycarbonylmethyl-2-thiouracilyl,
5-aminomethyl-2-thiouracilyl,
5-methylaminomethyluracilyl,
5-methylaminomethyl-2-thiouracilyl,
5-methylaminomethyl-2-selenouracilyl,
5-carbamoylmethyluracilyl,
5-carboxymethylaminomethyluracilyl,
5-carboxymethylaminomethyl-2-thiouracilyl,
3-methyluracilyl,
1-methyl-3-(3-amino-3-carboxypropyl) pseudouracilyl,
5-carboxymethyluracilyl,
5-methyldihydrouracilyl, or
3-methylpseudouracilyl.

A universal base can form base pairs with each of the natural DNA/RNA bases, exhibiting relatively little discrimination between them. In general, the universal bases are non-hydrogen bonding, hydrophobic, aromatic moieties which can stabilize e.g., duplex RNA or RNA-like molecules, via stacking interactions. A universal base can also include hydrogen bonding substituents. As used herein, a “universal base” can include anthracenes, pyrenes or any one of the following:

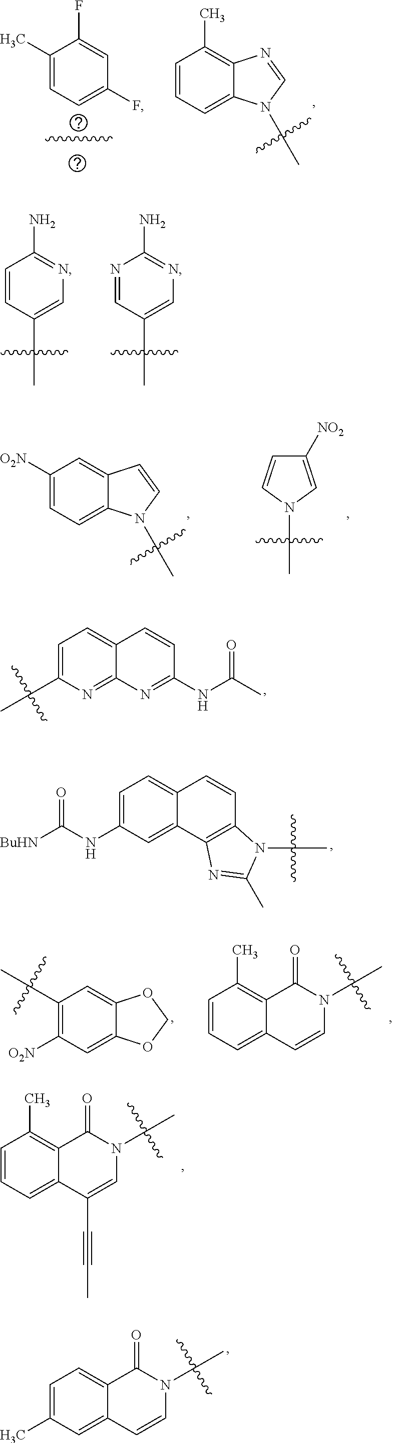

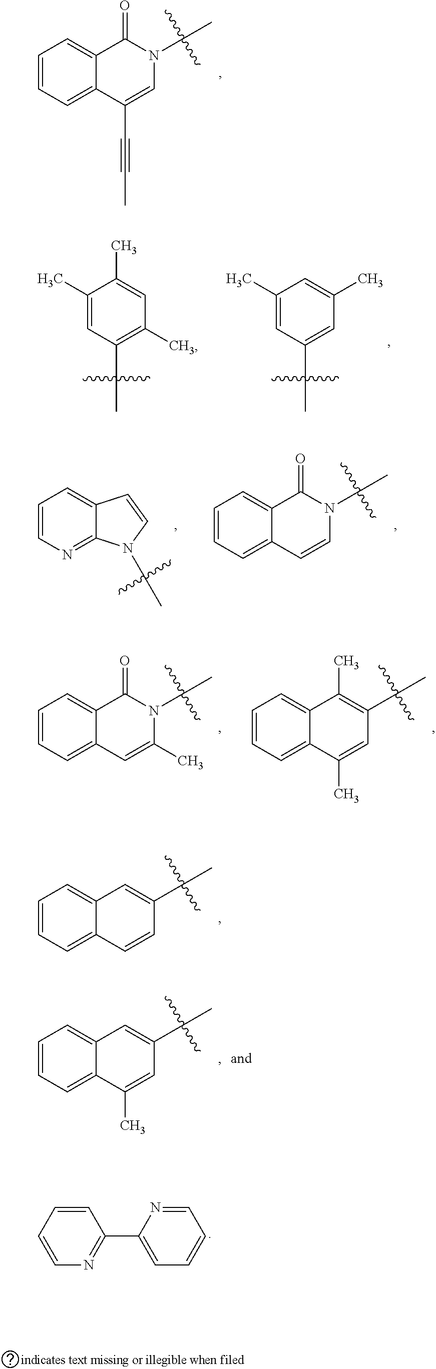

In some embodiments, B can form part of a tether that connects a ligand to the carrier. For example, the tether can be B—CH═CH—C(O)NH—(CH₂)₅—NHC(O)-LIGAND. In a preferred embodiment, the double bond is trans, and the ligand is a substituted or unsubstituted cholesterolyl radical (e.g., attached through the D-ring side chain or the C-3 hydroxyl); an aralkyl moiety having at least one sterogenic center and at least one substituent on the aryl portion of the aralkyl group; or a nucleobase. In certain embodiments, B, in the tether described above, is uracilyl or a universal base, e.g., an aryl moiety, e.g., phenyl, optionally having additional substituents, e.g., one or more fluoro groups. B can be substituted at any atom with the remainder of the tether.

X² can include “oxy” or “deoxy” substituents in place of the 2′-OH; or be a ligand or a tethered ligand.

Examples of “oxy”-substituents include alkoxy or aryloxy (OR, e.g., R═H, alkyl, cycloalkyl, aryl, aralkyl, heteroaryl, sugar, or protecting group); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O—PROTECTED AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙPROTECTED AMINE, (e.g., AMINE=NH₂; alkylamino, dialkylamino, heterocyclyl, arylamino, diaryl amino, heteroaryl amino, or diheteroaryl amino, ethylene diamine, polyamino), and orthoester. Amine protecting groups can include formyl, amido, benzyl, allyl, etc.

Preferred orthoesters have the general formula J. The groups R³¹ and R³² may be the same or different and can be any combination of the groups listed in FIG. 2B. A preferred orthoester is the “ACE” group, shown below as structure K.

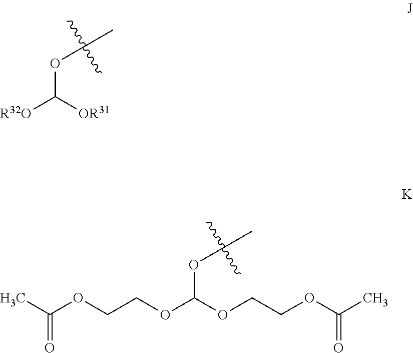

“Deoxy” substituents include hydrogen (i.e. deoxyribose sugars); halo (e.g., fluoro); protected amino (e.g. NH₂; alkylamino, dialkylamino, heterocyclyl, arylamino, diaryl amino, heteroaryl amino, diheteroaryl amino, or amino acid in which all amino are protected); fully protected polyamino (e.g., NH(CH₂CH₂NH)ₙCH₂CH₂-AMINE, wherein AMINE=NH₂; alkylamino, dialkylamino, heterocyclyl, arylamino, diaryl amino, heteroaryl amino, or diheteroaryl amino and all amino groups are protected), —NHC(O)R(R=alkyl, cycloalkyl, aryl, aralkyl, heteroaryl or sugar), cyano; alkyl-thio-alkyl; thioalkoxy; and alkyl, cycloalkyl, aryl, alkenyl and alkynyl, which may be optionally substituted with e.g., a protected amino functionality. Preferred substitutents are 2′-methoxyethyl, 2′-OCH3, 2′-O-allyl, 2′-C— allyl, and 2′-fluoro.

X³ is as described for OFG² above.

PG can be a triarylmethyl group (e.g., a dimethoxytrityl group) or Si(X^{5′})(X^{5″})(X^{5′″}) in which (X^{5′}), (X^{5″}), and (X^{5′″}) are as described elsewhere.

Sugar Replacement-Based Monomers, e.g., Ligand-Conjugated Monomers (Cyclic). Cyclic sugar replacement-based monomers, e.g., sugar replacement-based ligand-conjugated monomers, are also referred to herein as ribose replacement monomer subunit (RRMS) monomer compounds. Preferred carriers have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.)

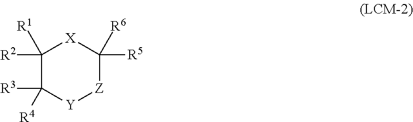

in which,

X is N(CO)R⁷, NR⁷ or CH₂;

Y is NR⁸, O, S, CR⁹R¹⁰;

Z is CR¹¹R¹² or absent;

Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b};

Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;

R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};

R⁸ is H or C₁-C₆ alkyl;

R¹³ is hydroxy, C₁-C₄ alkoxy, or halo;

R¹⁴ is NR^{c}R⁷;

R¹⁵ is C₁-C₆ alkyl optionally substituted with cyano, or C₂-C₆ alkenyl;

R¹⁶ is C₁-C₁₀ alkyl;

R¹⁷ is a liquid or solid phase support reagent;

L is —C(O)(CH₂)_{q}C(O)—, or —C(O)(CH₂)_{q}S—;

R^{a} is a protecting group, e.g., CAr₃; (e.g., a dimethoxytrityl group) or Si(X^{5′})(X^{5″})(X^{5′″}) in which (X^{5′}), (X^{5″}), and (X^{5′″}) are as described elsewhere.

R^{b} is P(O)(O⁻)H, P(OR¹⁵)N(R¹⁶)₂ or L-R¹⁷;

R^{c} is H or C₁-C₆ alkyl;

R^{d} is H or a ligand;

Each Ar is, independently, C₆-C₁₀ aryl optionally substituted with C₁-C₄ alkoxy;

n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D). OFG¹ is preferably attached to a primary carbon, e.g., an exocyclic alkylene

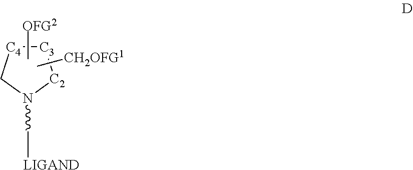

group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

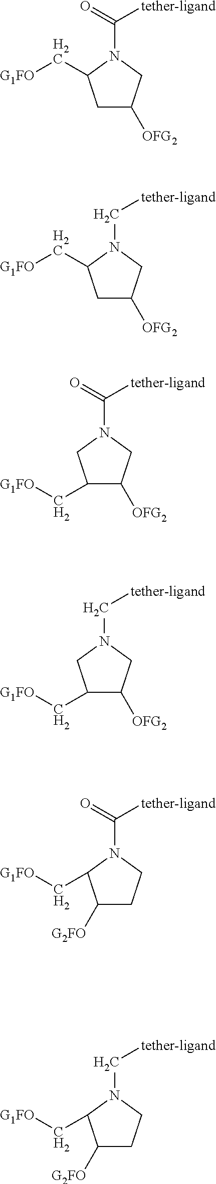

In certain embodiments, the carrier may be based on the piperidine ring system (E), e.g., X is N(CO)R⁷ or NR⁷, Y is CR⁹R¹⁰, and Z is CR¹¹R¹². OFG¹ is preferably

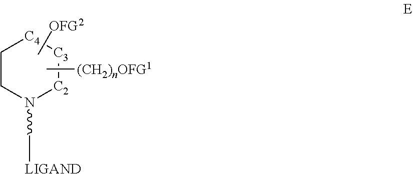

attached to a primary carbon, e.g., an exocyclic alkylene group, e.g., a methylene group (n=1) or ethylene group (n=2), connected to one of the carbons in the six-membered ring [—(CH₂)ₙOFG¹ in E], OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g.,

—(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹². OFG¹ is preferably

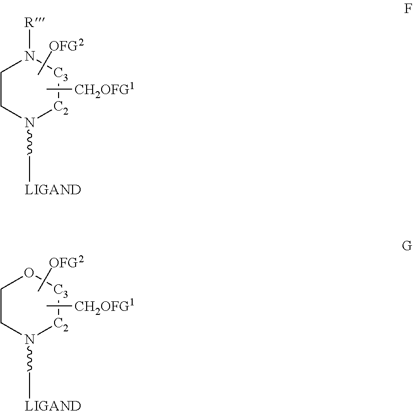

attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C₁-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1). OFG¹ is preferably attached to a primary carbon,

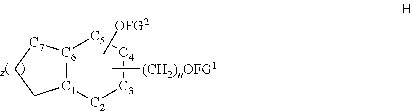

e.g., an exocyclic methylene group (n=1) or ethylene group (n=2) connected to one of C-2, C-3, C-4, or C-5 [-(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J). Thus, —(CH₂)ₙOFG¹ and OFG² may be cis or trans with respect to one another. Accordingly, all cis/trans isomers are expressly included. The monomers may also contain one or more asymmetric centers

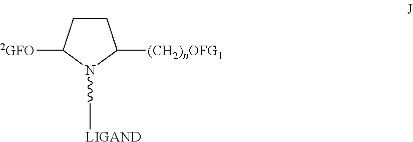

and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

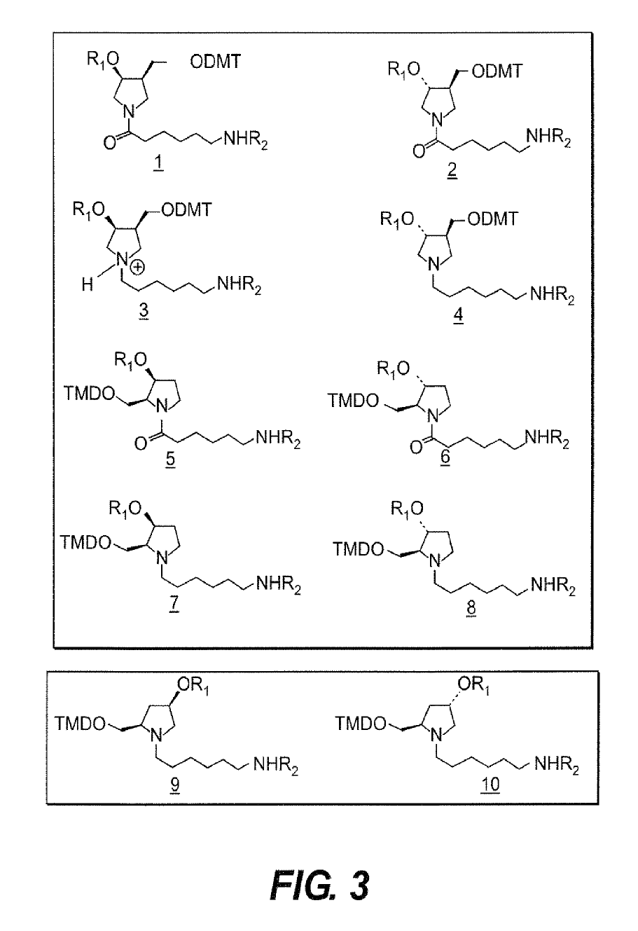

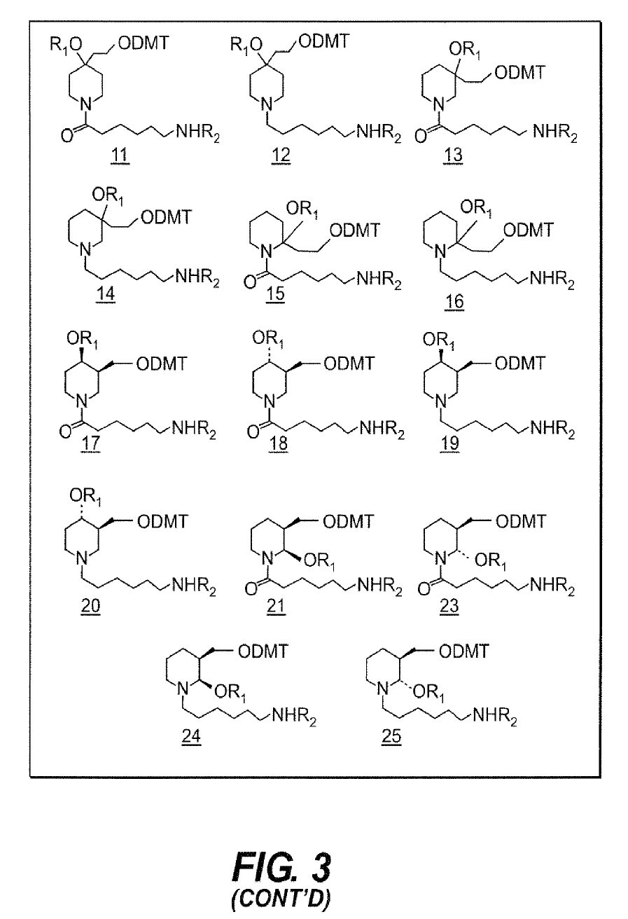

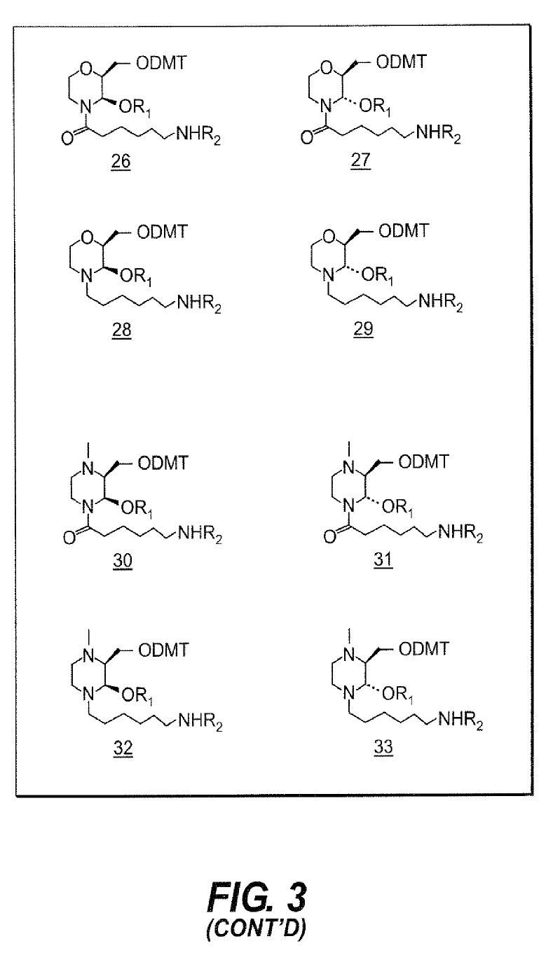

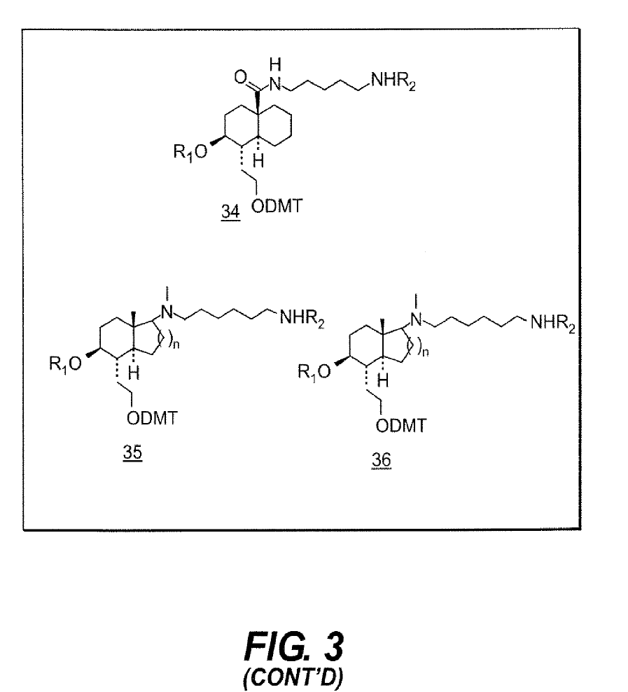

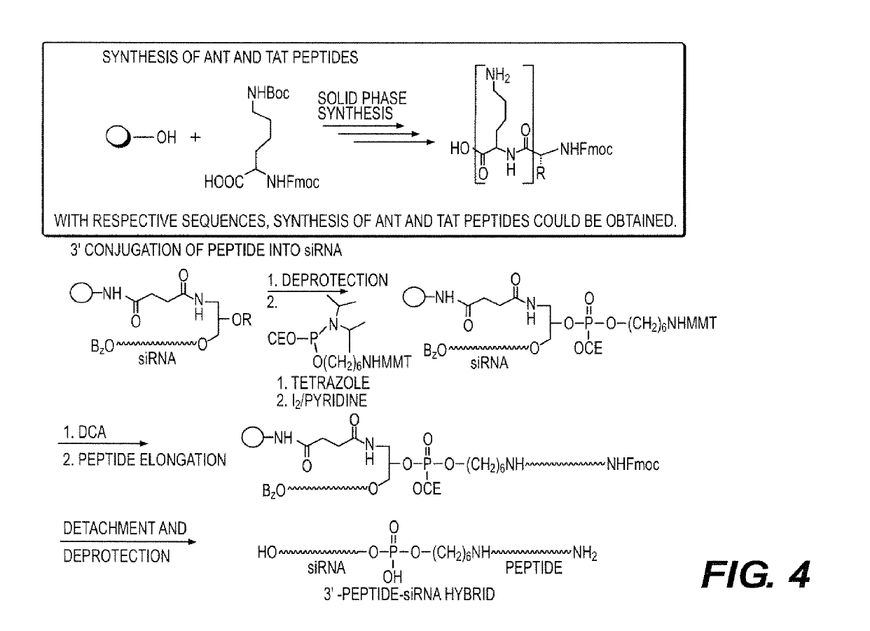

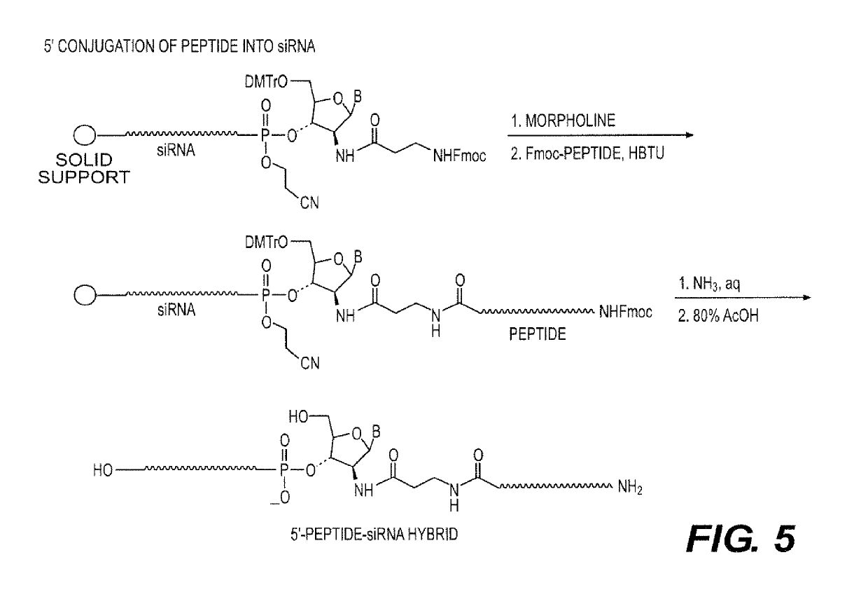

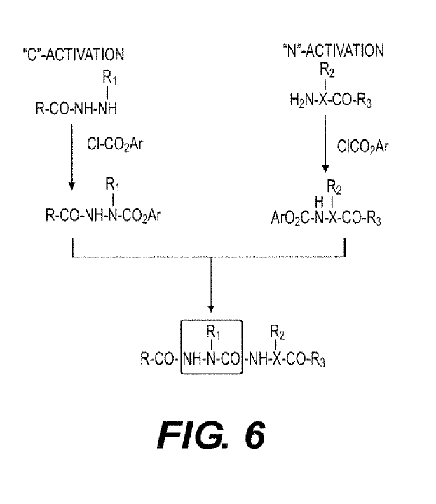

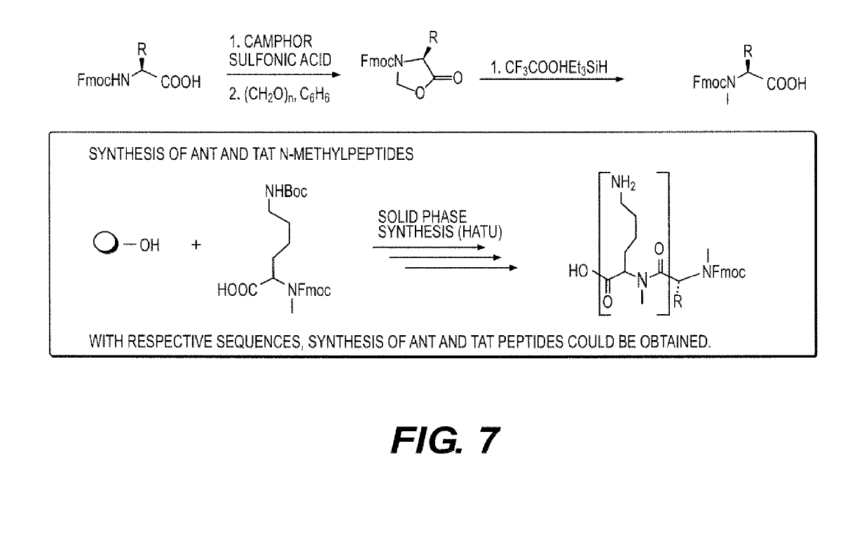

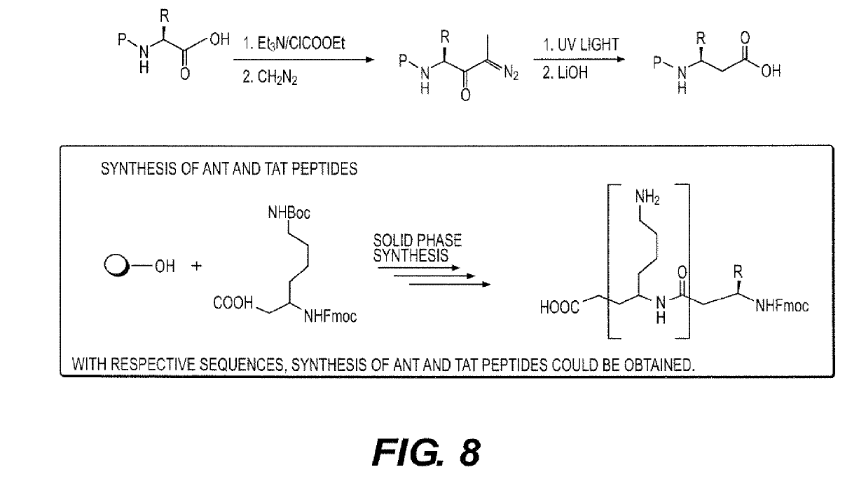

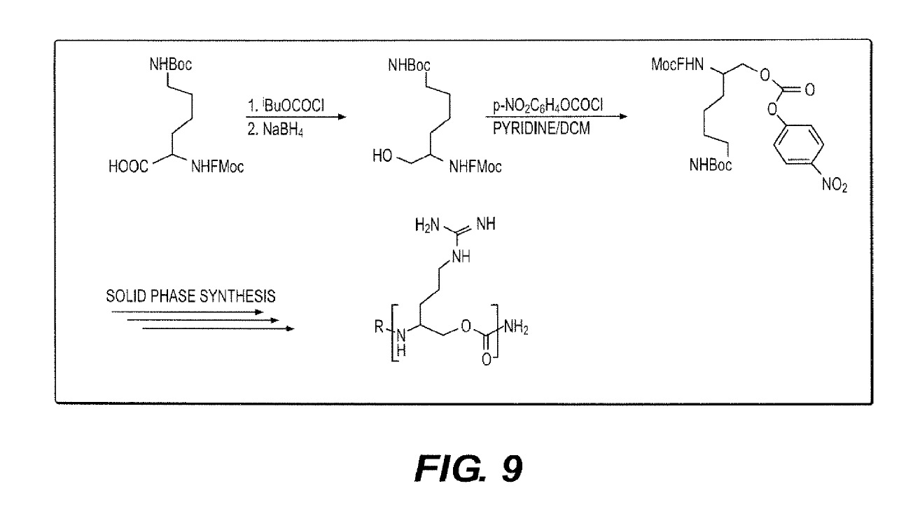

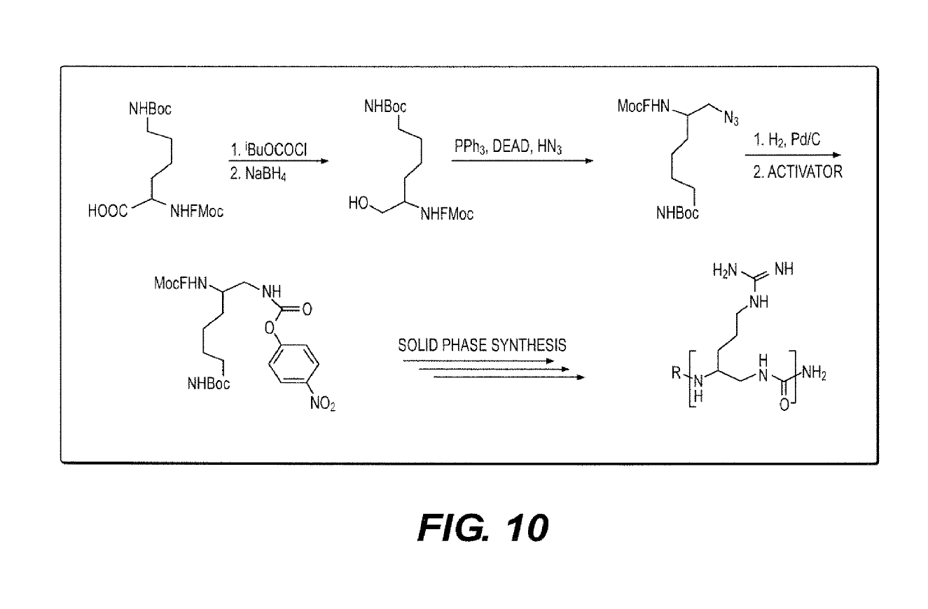

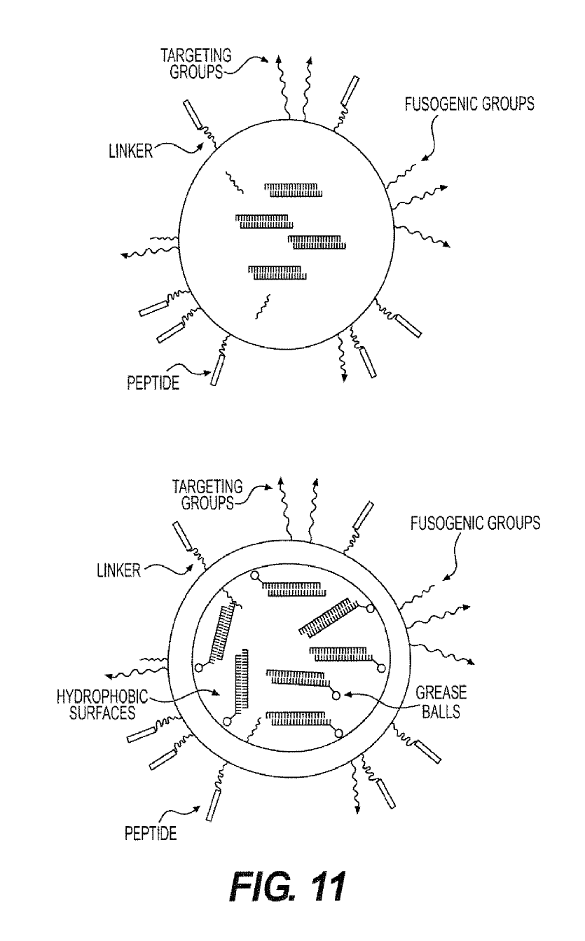

Representative cyclic, sugar replacement-based carriers are shown in FIG. 3. Sugar Replacement-Based Monomers (Acyclic). Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4 below.

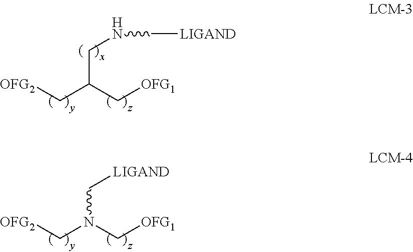

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Tethers. In certain embodiments, a moiety, e.g., a ligand may be connected indirectly to the carrier via the intermediacy of an intervening tether. Tethers are connected to the carrier at a tethering attachment point (TAP) and may include any C₁-C₁₀₀ carbon-containing moiety, (e.g. C₁-C₇₅, C₁-C₅₀, C₁-C₂₀, C₁-C₁₀; C₁, C₂, C₃, C₄, Cₛ, C₆, C₇, C₈, C₉, or C₁₀), preferably having at least one nitrogen atom. In preferred embodiments, the nitrogen atom forms part of a terminal amino or amido (NHC(O)—) group on the tether, which may serve as a connection point for the ligand. Preferred tethers (underlined) include TAP-(CH₂)ₙNH—; TAP-C(O)(CH₂)ₙNH—; TAP-NR″″(CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙNH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)—C(O)O(LIGAND); TAP-(CH₂)ₙ(LIGAND); or TAP-(CH₂)ₙ—NH—C(O)(LIGAND). In some embodiments, amino terminated tethers (e.g., NH₂, ONH₂, NH₂NH₂) can form an imino bond (i.e., C═N) with the ligand. In some embodiments, amino terminated tethers (e.g., NH₂, ONH₂, NH₂NH₂) can acylated, e.g., with C(O)CF₃.

In some embodiments, the tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments the tether may include an electrophilic moiety, preferably at the terminal position of the tether. Preferred electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)C(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O)OC6F5; or TAP-NR″″(CH₂)ₙC(O)OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the tether.

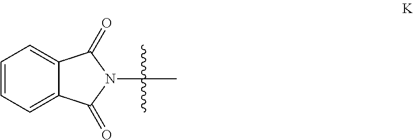

In other embodiments, other protected amino groups can be at the terminal position of the tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the tethers described herein may further include one or more additional linking groups, e.g., —O—(CH₂)ₙ—, —(CH₂)ₙ—SS—, —(CH₂)ₙ—, or —(CH═CH)—.

Tethered Ligands. A wide variety of entities, e.g., ligands, can be tethered to an iRNA agent, e.g., to the carrier of a ligand-conjugated monomer subunit. Examples are described below in the context of a ligand-conjugated monomer subunit but that is only preferred, entities can be coupled at other points to an iRNA agent.

Preferred moieties are ligands, which are coupled, preferably covalently, either directly or indirectly via an intervening tether, to the carrier. In preferred embodiments, the ligand is attached to the carrier via an intervening tether. As discussed above, the ligand or tethered ligand may be present on the ligand-conjugated monomer \ when the ligand-conjugated monomer is incorporated into the growing strand. In some embodiments, the ligand may be incorporated into a “precursor” ligand-conjugated monomer subunit after a “precursor” ligand-conjugated monomer subunit has been incorporated into the growing strand. For example, a monomer having, e.g., an amino-terminated tether, e.g., TAP-(CH₂)ₙNH₂ may be incorporated into a growing sense or antisense strand. In a subsequent operation, i.e., after incorporation of the precursor monomer subunit into the strand, a ligand having an electrophilic group, e.g., a pentafluorophenyl ester or aldehyde group, can subsequently be attached to the precursor ligand-conjugated monomer by coupling the electrophilic group of the ligand with the terminal nucleophilic group of the precursor ligand-conjugated monomer subunit tether.

In preferred embodiments, a ligand alters the distribution, targeting or lifetime of an iRNA agent into which it is incorporated. In preferred embodiments a ligand provides an enhanced affinity for a selected target, e.g, molecule, cell or cell type, compartment, e.g., a cellular or organ compartment, tissue, organ or region of the body, as, e.g., compared to a species absent such a ligand.

Preferred ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., cholesterol, uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, vitamin E, biotin, pyridoxal), carbohydrates, proteins, protein binding agents, integrin targeting molecules, polycationics, peptides, polyamines, and peptide mimics.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

In some embodiments, the ligand can be one of the following triterpenes:

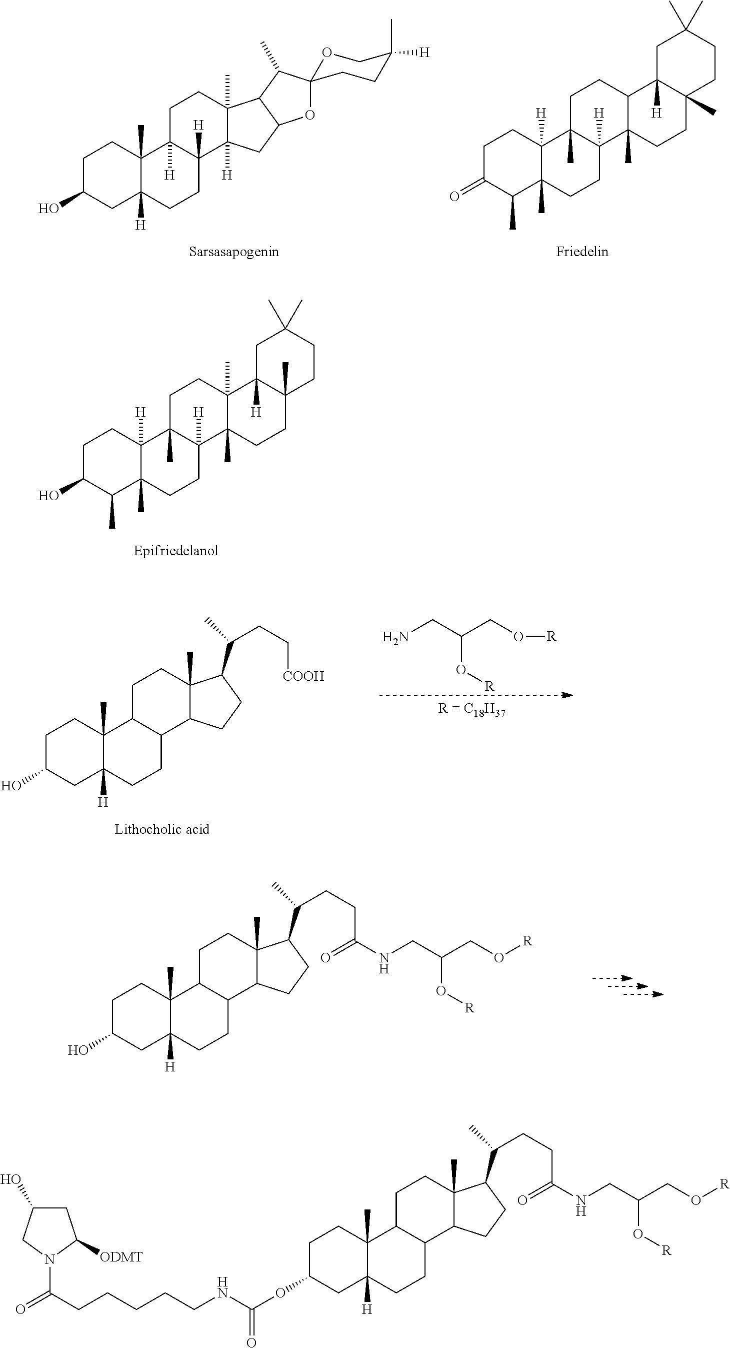

In some embodiments, the ligand can be substituted or unsubstituted cholesterol, or a stereoisomer thereof or one of the following steroids:

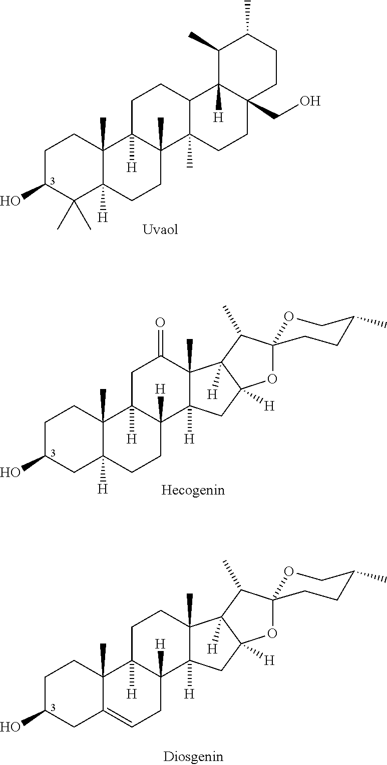

iRNA AGENT STRUCTURE The monomers described herein can be used to make oligonucleotides which are useful as iRNA agents, e.g., RNA molecules, (double-stranded; single-stranded) that mediate RNAi, e.g., with respect to an endogenous gene of a subject or to a gene of a pathogen. In most cases the iRNA agent will incorporate momomers described herein together with naturally occurring nucleosides or nucleotides or with other modified nucleosides or nucleotides. The modified monomers can be present at any position in the iRNA agent, e.g., at the terminii or in the middle region of an iRNA agent or in a duplex region or in an unpaired region. In a preferred embodiment iRNA agent can have any architecture, e.g., architecture described herein. E.g., it can be incorporated into an iRNA agent having an overhang structure, a hairpin or other single strand structure or a two-strand structure, as described herein.

An “RNA agent” as used herein, is an unmodified RNA, modified RNA, or nucleoside surrogate, all of which are defined herein (see, e.g., the section below entitled RNA Agents). While numerous modified RNAs and nucleoside surrogates are described, preferred examples include those which have greater resistance to nuclease degradation than do unmodified RNAs. Preferred examples include those which have a 2′ sugar modification, a modification in a single strand overhang, preferably a 3′ single strand overhang, or, particularly if single stranded, a 5′ modification which includes one or more phosphate groups or one or more analogs of a phosphate group.

An “iRNA agent,” as used herein, is an RNA agent which can, or which can be cleaved into an RNA agent which can, stimulate or inhibit an immune response, or have no effect on an immune response. An iRNA agent may also down regulate the expression of a target gene, preferably an endogenous or pathogen target RNA. While not wishing to be bound by theory, an iRNA agent that down regulates expression of a target gene may act by one or more of a number of mechanisms, including post-transcriptional cleavage of a target mRNA (sometimes referred to in the art as RNAi), or pre-transcriptional or pre-translational mechanisms. An iRNA agent can include a single strand or can include more than one strands, e.g., it can be a double stranded iRNA agent. If the iRNA agent is a single strand it is particularly preferred that it include a 5′ modification which includes one or more phosphate groups or one or more analogs of a phosphate group.

For ease of exposition the term nucleotide or ribonucleotide is sometimes used herein in reference to one or more monomeric subunits of an RNA agent. It will be understood herein that the usage of the term “ribonucleotide” or “nucleotide” can, in the case of a modified RNA or nucleotide surrogate, also refer to a modified nucleotide, or surrogate replacement moiety at one or more positions.

As discussed elsewhere herein, an iRNA agent will often be modified or include nucleoside surrogates in addition to the ribose replacement modification subunit (RRMS). Single stranded regions of an iRNA agent will often be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an iRNA agent, e.g., against exonucleases, or to favor the antisense sRNA agent to enter into RISC are also favored. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

An iRNA agent will preferably have one or more of the following properties:
(1) it will be of the Formula 1, 2, 3, or 4 set out in the RNA Agent section below;
(2) if single stranded it will have a 5′ modification which includes one or more phosphate groups or one or more analogs of a phosphate group;
(3) it will, despite modifications, even to a very large number, or all of the nucleosides, have an antisense strand that can present bases (or modified bases) in the proper three dimensional framework so as to be able to form correct base pairing and form a duplex structure with a homologous target RNA which is sufficient to allow down regulation of the target, e.g., by cleavage of the target RNA;
(4) it will, despite modifications, even to a very large number, or all of the nucleosides, still have “RNA-like” properties, i.e., it will possess the overall structural, chemical and physical properties of an RNA molecule, even though not exclusively, or even partly, of ribonucleotide-based content. For example, an iRNA agent can contain, e.g., a sense and/or an antisense strand in which all of the nucleotide sugars contain e.g., 2′ fluoro in place of 2′ hydroxyl. This deoxyribonucleotide-containing agent can still be expected to exhibit RNA-like properties. While not wishing to be bound by theory, the electronegative fluorine prefers an axial orientation when attached to the C2′ position of ribose. This spatial preference of fluorine can, in turn, force the sugars to adopt a C_{3′}-endo pucker. This is the same puckering mode as observed in RNA molecules and gives rise to the RNA-characteristic A-family-type helix. Further, since fluorine is a good hydrogen bond acceptor, it can participate in the same hydrogen bonding interactions with water molecules that are known to stabilize RNA structures. (Generally, it is preferred that a modified moiety at the 2′ sugar position will be able to enter into H-bonding which is more characteristic of the OH moiety of a ribonucleotide than the H moiety of a deoxyribonucleotide. A preferred iRNA agent will: exhibit a C_{3′}-endo pucker in all, or at least 50, 75, 80, 85, 90, or 95% of its sugars; exhibit a C_{3′}-endo pucker in a sufficient amount of its sugars that it can give rise to a the RNA-characteristic A-family-type helix; will have no more than 20, 10, 5, 4, 3, 2, or 1 sugar which is not a C_{3′}-endo pucker structure. These limitations are particularly preferably in the antisense strand;
(4) regardless of the nature of the modification, and even though the RNA agent can contain deoxynucleotides or modified deoxynucleotides, particularly in overhang or other single strand regions, it is preferred that DNA molecules, or any molecule in which more than 50, 60, or 70% of the nucleotides in the molecule, or more than 50, 60, or 70% of the nucleotides in a duplexed region are deoxyribonucleotides, or modified deoxyribonucleotides which are deoxy at the 2′ position, are excluded from the definition of RNA agent.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents are preferably antisense with regard to the target molecule. In preferred embodiments single strand iRNA agents are 5′ phosphorylated or include a phosphoryl analog at the 5′ prime terminus 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl (MeOCH2-), ethoxymethyl, etc., e.g. RP(OH)(O)—O-5′-). (These modifications can also be used with the antisense strand of a double stranded iRNA.)

It may be desirable to modify one or both of the antisense and sense strands of a double strand iRNA agent. In some cases they will have the same modification or the same class of modification but in other cases the sense and antisense strand will have different modifications, e.g., in some cases it is desirable to modify only the sense strand. It may be desirable to modify only the sense strand, e.g., to inactivate it, e.g., the sense strand can be modified in order to inactivate the sense strand and prevent formation of an active sRNA/protein or RISC. This can be accomplished by a modification which prevents 5′-phosphorylation of the sense strand, e.g., by modification with a 5′-O-methyl ribonucleotide (see Nykänen et al., (2001) ATP requirements and small interfering RNA structure in the RNA interference pathway. Cell 107, 309-321.) Other modifications which prevent phosphorylation can also be used, e.g., simply substituting the 5′-OH by H rather than O-Me. Alternatively, a large bulky group may be added to the 5′-phosphate turning it into a phosphodiester linkage, though this may be less desirable as phosphodiesterases can cleave such a linkage and release a functional sRNA 5′-end. Antisense strand modifications include 5′ phosphorylation as well as any of the other 5′ modifications discussed herein, particularly the 5′ modifications discussed above in the section on single stranded iRNA molecules.

In some cases the sense and the antisense strands will include different modifications. Multiple different modifications can be included on the sense and antisense strand. The modifications on each strand may differ from each other, and may also differ from the various modifications on the other strand. For example, the sense strand may have a modification, e.g., a modification described herein, and the antisense strand may have a different modification described herein. In other cases, one strand, such as the sense strand may have two different modifications, and the antisense strand may include a modification that differs from the at least two modifications on the sense strand.

It is preferred that the sense and antisense strands be chosen such that the ds iRNA agent includes a single strand or unpaired region at one or both ends of the molecule. Thus, a ds iRNA agent contains sense and antisense strands, preferable paired to contain an overhang, e.g., one or two 5′ or 3′ overhangs but preferably a 3′ overhang of 2-3 nucleotides. Most embodiments will have a 3′ overhang. Preferred sRNA agents will have single-stranded overhangs, preferably 3′ overhangs, of 1 or preferably 2 or 3 nucleotides in length at each end. The overhangs can be the result of one strand being longer than the other, or the result of two strands of the same length being staggered. 5′ ends are preferably phosphorylated.

Preferred lengths for the duplexed region is between 15 and 30, e.g., 18, 19, 20, 21, 22, 23, 25, 27, and 29 nucleotides in length, e.g., in the sRNA agent range discussed above. sRNA agents can resemble in length and structure the natural Dicer processed products from long dsRNAs. Embodiments in which the two strands of the sRNA agent are linked, e.g., covalently linked are also included. Hairpin, or other single strand structures which provide the required double stranded region, and preferably a 3′ overhang are also within the invention.

As nucleic acids are polymers of subunits or monomers, many of the modifications described below occur at a position which is repeated within a nucleic acid, e.g., a modification of a base, or a phosphate moiety, or the a non-linking O of a phosphate moiety. In some cases the modification will occur at all of the subject positions in the nucleic acid but in many, and infact in most cases it will not. By way of example, a modification may only occur at a 3′ or 5′ terminal position, may only occur in a terminal regions, e.g. at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. The 5′ end or ends can be phosphorylated.

In some embodiments it is particularly preferred, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

Modifications and nucleotide surrogates are discussed below.

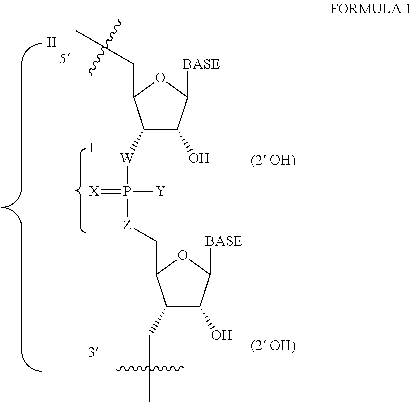

The scaffold presented above in Formula 1 represents a portion of a ribonucleic acid. The basic components are the ribose sugar, the base, the terminal phosphates, and phosphate internucleotide linkers. Where the bases are naturally occurring bases, e.g., adenine, uracil, guanine or cytosine, the sugars are the unmodified 2′ hydroxyl ribose sugar (as depicted) and W, X, Y, and Z are all O, Formula 1 represents a naturally occurring unmodified oligoribonucleotide.

Unmodified oligoribonucleotides may be less than optimal in some applications, e.g., unmodified oligoribonucleotides can be prone to degradation by e.g., cellular nucleases. Nucleases can hydrolyze nucleic acid phosphodiester bonds. However, chemical modifications to one or more of the above RNA components can confer improved properties, and, e.g., can render oligoribonucleotides more stable to nucleases. Umodified oligoribonucleotides may also be less than optimal in terms of offering tethering points for attaching ligands or other moieties to an iRNA agent.

Modified nucleic acids and nucleotide surrogates can include one or more of:

(i) alteration, e.g., replacement, of one or both of the non-linking (X and Y) phosphate oxygens and/or of one or more of the linking (W and Z) phosphate oxygens (When the phosphate is in the terminal position, one of the positions W or Z will not link the phosphate to an additional element in a naturally occurring ribonucleic acid. However, for simplicity of terminology, except where otherwise noted, the W position at the 5′ end of a nucleic acid and the terminal Z position at the 3′ end of a nucleic acid, are within the term “linking phosphate oxygens” as used herein);

(ii) alteration, e.g., replacement, of a constituent of the ribose sugar, e.g., of the 2′ hydroxyl on the ribose sugar, or wholesale replacement of the ribose sugar with a structure other than ribose, e.g., as described herein;

(iii) wholesale replacement of the phosphate moiety (bracket I) with “dephospho” linkers;

(iv) modification or replacement of a naturally occurring base;

(v) replacement or modification of the ribose-phosphate backbone (bracket II);

(vi) modification of the 3′ end or 5′ end of the RNA, e.g., removal, modification or replacement of a terminal phosphate group or conjugation of a moiety, e.g. a fluorescently labeled moiety, to either the 3′ or 5′ end of RNA.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species. For example, any phosphoroamidate (replacement of a nonlinking oxygen with nitrogen) would be represented by X═O and Y═N in the above figure.

Specific modifications are discussed in more detail below.

The Phosphate Group. The phosphate group is a negatively charged species. The charge is distributed equally over the two non-linking oxygen atoms (i.e., X and Y in Formula 1 above). However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. Unlike the situation where only one of X or Y is altered, the phosphorus center in the phosphorodithioates is achiral which precludes the formation of oligoribonucleotides diastereomers. Diastereomer formation can result in a preparation in which the individual diastereomers exhibit varying resistance to nucleases. Further, the hybridization affinity of RNA containing chiral phosphate groups can be lower relative to the corresponding unmodified RNA species. Thus, while not wishing to be bound by theory, modifications to both X and Y which eliminate the chiral center, e.g. phosphorodithioate formation, may be desirable in that they cannot produce diastereomer mixtures. Thus, X can be any one of S, Se, B, C, H, N, or OR (R is alkyl or aryl). Thus Y can be any one of S, Se, B, C, H, N, or OR (R is alkyl or aryl). Replacement of X and/or Y with sulfur is preferred.

The phosphate linker can also be modified by replacement of a linking oxygen (i.e., W or Z in Formula 1) with nitrogen (bridged phosphoroamidates), sulfur (bridged phosphorothioates) and carbon (bridged methylenephosphonates). The replacement can occur at a terminal oxygen (position W (3′) or position Z (5′). Replacement of W with carbon or Z with nitrogen is preferred.

Candidate agents can be evaluated for suitability as described below.

The Sugar Group. A modified RNA can include modification of all or some of the sugar groups of the ribonucleic acid. E.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′ alkoxide ion. The 2′ alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Preferred substitutents are 2′-methoxyethyl, 2′-OCH3, 2′-O-allyl, 2′-C— allyl, and 2′-fluoro.

The sugar group can also contain one or more carbons that possess the opposite stereochemical configuration than that of the corresponding carbon in ribose. Thus, a modified RNA can include nucleotides containing e.g., arabinose, as the sugar.

Modified RNAs can also include “abasic” sugars, which lack a nucleobase at C-1′. These abasic sugars can also be further contain modifications at one or more of the constituent sugar atoms.

To maximize nuclease resistance, the 2′ modifications can be used in combination with one or more phosphate linker modifications (e.g., phosphorothioate). The so-called “chimeric” oligonucleotides are those that contain two or more different modifications.

The modificaton can also entail the wholesale replacement of a ribose structure with another entity at one or more sites in the iRNA agent.

Candidate modifications can be evaluated as described below.

Replacement of the Phosphate Group. The phosphate group can be replaced by non-phosphorus containing connectors (cf. Bracket I in Formula 1 above). While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

Candidate modifications can be evaluated as described below.

Replacement of Ribophosphate Backbone. Oligonucleotide-mimicking scaffolds can also be constructed wherein the phosphate linker and ribose sugar are replaced by nuclease resistant nucleoside or nucleotide surrogates (see Bracket II of Formula 1 above). While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone.

Examples include the mophilino, cyclobutyl, pyrrolidine and peptide nucleic acid (PNA) nucleoside surrogates. A preferred surrogate is a PNA surrogate.

Candidate modifications can be evaluated as described below.

Terminal Modifications. The 3′ and 5′ ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a spacer. The terminal atom of the spacer can connect to or replace the linking atom of the phosphate group or the C-3′ or C-5′ O, N, S or C group of the sugar. Alternatively, the spacer can connect to or replace the terminal atom of a nucleotide surrogate (e.g., PNAs). These spacers or linkers can include e.g., —(CH₂)ₙ—, —(CH₂)ₙN—, —(CH₂)ₙO—, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents. When a spacer/phosphate-functional molecular entity-spacer/phosphate array is interposed between two strands of iRNA agents, this array can substitute for a hairpin RNA loop in a hairpin-type RNA agent. The 3′ end can be an —OH group. While not wishing to be bound by theory, it is believed that conjugation of certain moieties can improve transport, hybridization, and specificity properties. Again, while not wishing to be bound by theory, it may be desirable to introduce terminal alterations that improve nuclease resistance. Other examples of terminal modifications include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can be added for a number of reasons, including as discussed elsewhere herein to modulate activity or to modulate resistance to degradation. Terminal modifications useful for modulating activity include modification of the 5′ end with phosphate or phosphate analogs. E.g., in preferred embodiments iRNA agents, especially antisense strands, are 5′ phosphorylated or include a phosphoryl analog at the 5′ prime terminus 5′-phosphate modifications include those which are compatible with RISC mediated gene silencing. Suitable modifications include: 5′-monophosphate ((HO)2(O)P—O-5′); 5′-diphosphate ((HO)₂(O)P—O—P(HO)(O)—O-5′); 5′-triphosphate ((HO)2(O)P-β-(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl (MeOCH2-), ethoxymethyl, etc., e.g. RP(OH)(O)—O-5′-).

Terminal modifications can also be useful for monitoring distribution, and in such cases the preferred groups to be added include fluorophores, e.g., fluorscein or an Alexa dye, e.g., Alexa 488. Terminal modifications can also be useful for enhancing uptake, useful modifications for this include cholesterol. Terminal modifications can also be useful for cross-linking an RNA agent to another moiety; modifications useful for this include mitomycin C.

Candidate modifications can be evaluated as described below.

The Bases. Adenine, guanine, cytosine and uracil are the most common bases found in RNA. These bases can be modified or replaced to provide RNA's having improved properties. E.g., nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases, e.g., “unusual bases” and “universal bases” described herein, can be employed. Examples include without limitation 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

Generally, base changes are less preferred for promoting stability, but they can be useful for other reasons, e.g., some, e.g., 2,6-diaminopurine and 2 amino purine, are fluorescent. Modified bases can reduce target specificity. This should be taken into consideration in the design of iRNA agents.

General References. The oligoribonucleotides and oligoribonucleosides used in accordance with this invention may be with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3,2′-O-Methyloligoribonucleotide-s:synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein.

Modification described in WO 00/44895, WO01/75164, or WO02/44321 can be used herein.

The disclosure of all publications, patents, and published patent applications listed herein are hereby incorporated by reference.

Phosphate Group References. The preparation of phosphinate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenephosphonate oligoribonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Lett. 1989, 30, 4693.

Sugar Group References. Modifications to the 2′ modifications can be found in Manoharan, Biochimica et Biophysica Acta 1489:117-130, 1999; Verma, S. et al. Annu. Rev. Biochem. 67:99-134, 1998 and references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310). iRNA-specific chemical modifications are described in Manoharan, Current Opinion in Chemical Biology 8:570-579, 2004.

Replacement of the Phosphate Group References. Methylenemethylimino linked oligoribonucleosides, also identified herein as MMI linked oligoribonucleosides, methylenedimethylhydrazo linked oligoribonucleosides, also identified herein as MDH linked oligoribonucleosides, and methylenecarbonylamino linked oligonucleosides, also identified herein as amide-3 linked oligoribonucleosides, and methyleneaminocarbonyl linked oligonucleosides, also identified herein as amide-4 linked oligoribonucleosides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleosides can be prepared as is described in U.S. Pat. Nos. 5,264,562 and 5,264,564. Ethylene oxide linked oligoribonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 1 1972, 1991. Carbamate replacements are described in Stirchak, E. P. Nucleic Acids Res. 1989, 17, 6129.

Replacement of the Phosphate-Ribose Backbone References. Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. No. 5,539,083.

Terminal Modification References. Terminal modifications are described in Manoharan, M. et al. Antisense and Nucleic Acid Drug Development 12, 103-128 (2002) and references therein.

Bases References. N-2 substitued purine nucleoside amidites can be prepared as is described in U.S. Pat. No. 5,459,255. 3-Deaza purine nucleoside amidites can be prepared as is described in U.S. Pat. No. 5,457,191. 5,6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908. Additional references can be disclosed in the above section on base modifications.

Preferred IRNA Agents Preferred RNA agents have the following structure (see Formula 2 below):

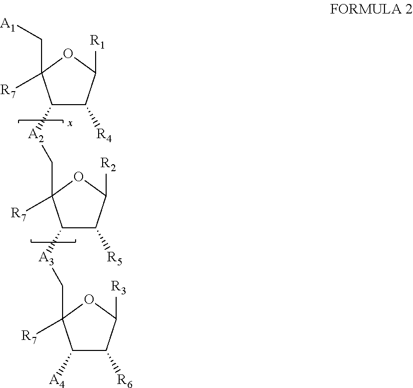

Referring to Formula 2 above, R¹, R², and R³ are each, independently, H, (i.e. abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases.

R⁴, R⁵, and R⁶ are each, independently, OR⁸, O(CH₂CH₂O)ₘCH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂NHR⁹; NHC(O)R⁸; cyano; mercapto, SR⁸; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, or ureido; or R⁴, R⁵, or R⁶ together combine with R⁷ to form an [—O—CH₂—] covalently bound bridge between the sugar 2′ and 4′ carbons.

A¹ is:

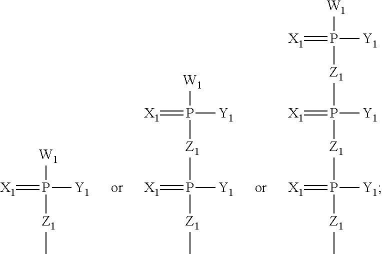

H; OH; OCH₃; W¹; an abasic nucleotide; or absent;

(a preferred A1, especially with regard to anti-sense strands, is chosen from 5′-monophosphate ((HO)₂(O)P—O-5′), 5′-diphosphate ((HO)₂(O)P—O—P(HO)(O)—O-5′), 5′-triphosphate ((HO)₂(O)P—O—(HO)(O)P—O—P(HO)(O)—O-5′), 5′-guanosine cap (7-methylated or non-methylated) (7m-G-O-5′-(HO)(O)P—O—(HO)(O)P—O—P(HO)(O)—O-5′), 5′-adenosine cap (Appp), and any modified or unmodified nucleotide cap structure (N—O-5′-(HO)(O)P-β-(HO)(O)P—O—P(HO)(O)—O-5′), 5′-monothiophosphate (phosphorothioate; (HO)₂(S)P—O-5′), 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, (OH)₂(O)P-5′-CH₂—), 5′-alkyletherphosphonates (R=alkylether=methoxymethyl (MeOCH₂—), ethoxymethyl, etc., e.g. RP(OH)(O)—O-5′-)).

A² is:

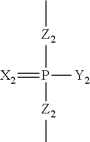

A³ is:

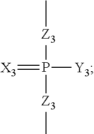

and

A⁴ is:

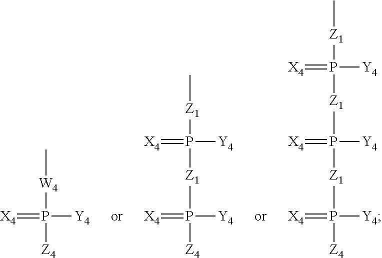

H; Z⁴; an inverted nucleotide; an abasic nucleotide; or absent.

W¹ is OH, (CH₂)ₙR¹⁰, (CH₂)ₙNHR¹⁰, (CH₂)ₙOR¹⁰, (CH₂)ₙSR¹⁰; O(CH₂)ₙR¹⁰; O(CH₂)ₙOR¹⁰, O(CH₂)ₙNR¹⁰, O(CH₂)ₙSR¹⁰; O(CH₂)ₙSS(CH₂)ₙOR¹⁰, O(CH₂)ₙC(O)OR¹⁰, NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰O(CH₂CH₂O)ₘCH₂CH₂OR¹⁰; O(CH₂CH₂O)ₘCH₂CH₂NHR¹⁰, NH(CH₂CH₂NH)ₘCH₂CH₂NHR¹⁰; Q-R¹⁰, O-Q-R¹⁰N-Q-R¹⁰, S-Q-R¹⁰ or —O—. W⁴ is O, CH₂, NH, or S.

X¹, X², X³, and X⁴ are each, independently, O or S.

Y¹, Y², Y³, and Y⁴ are each, independently, OH, O⁻, OR⁸, S, Se, BH₃⁻, H, NHR⁹, N(R⁹)₂ alkyl, cycloalkyl, aralkyl, aryl, or heteroaryl, each of which may be optionally substituted.

Z¹, Z², and Z³ are each independently O, CH₂, NH, or S. Z⁴ is OH, (CH₂)ₙR¹⁰, (CH₂)ₙNHR¹⁰, (CH₂)ₙOR¹⁰, (CH₂)ₙ SR¹⁰; O(CH₂)ₙR¹⁰; O(CH₂)ₙOR¹⁰, O(CH₂)ₙNR¹⁰, O(CH₂)ₙSR¹⁰, O(CH₂)ₙSS(CH₂)ₙOR¹⁰, O(CH₂)ₙC(O)OR¹⁰; NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰O(CH₂CH₂O)ₘCH₂CH₂OR¹⁰, O(CH₂CH₂O)ₘCH₂CH₂NHR¹⁰, NH(CH₂CH₂NH)ₘCH₂CH₂NHR¹⁰; Q-R¹⁰, O-Q-R¹⁰N-Q-R¹⁰, S-Q-R¹⁰.

x is 5-100, chosen to comply with a length for an RNA agent described herein.

R⁷ is H; or is together combined with R⁴, R⁵, or R⁶ to form an [—O—CH₂—] covalently bound bridge between the sugar 2′ and 4′ carbons.

R⁸ is alkyl, cycloalkyl, aryl, aralkyl, heterocyclyl, heteroaryl, amino acid, or sugar; R⁹ is NH₂, alkylamino, dialkylamino, heterocyclyl, arylamino, diaryl amino, heteroaryl amino, diheteroaryl amino, or amino acid; and R¹⁰ is H; fluorophore (pyrene, TAMRA, fluorescein, Cy3 or Cy5 dyes); sulfur, silicon, boron or ester protecting group; intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipohilic carriers (cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g PEG-40K), MPEG, [MPEG]₂, polyamino; alkyl, cycloalkyl, aryl, aralkyl, heteroaryl; radiolabelled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles); or an RNA agent. m is 0-1,000,000, and n is 0-20. Q is a spacer selected from the group consisting of abasic sugar, amide, carboxy, oxyamine, oxyimine, thioether, disulfide, thiourea, sulfonamide, or morpholino, biotin or fluorescein reagents.

Preferred RNA agents in which the entire phosphate group has been replaced have the following structure (see Formula 3 below):

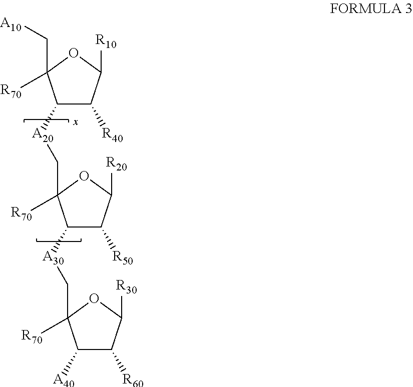

Referring to Formula 3, A¹⁰-A⁴⁰ is L-G-L; A¹⁰ and/or A⁴⁰ may be absent, in which L is a linker, wherein one or both L may be present or absent and is selected from the group consisting of CH₂(CH₂)_{g}; N(CH₂)_{g}; O(CH₂)_{g}; S(CH₂)_{g}. G is a functional group selected from the group consisting of siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino

R¹⁰, R²⁰, and R³⁰ are each, independently, H, (i.e. abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases.

R⁴⁰, R⁵⁰, and R⁶⁰ are each, independently, OR⁸, O(CH₂CH₂O)ₘCH₂CH₂OR⁸; OCH₂)ₙR⁹; O(CH₂)ₙOR⁹, H; halo; NH₂; NHR⁸; N(R⁸)₂; NH(CH₂CH₂NH)ₘCH₂CH₂R⁹; NHC(O)R⁸; cyano; mercapto, SR⁷; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, and ureido groups; or R⁴⁰, R⁵⁰, or R⁶⁰ together combine with R⁷⁰ to form an [—O—CH₂—] covalently bound bridge between the sugar 2′ and 4′ carbons.

x is 5-100 or chosen to comply with a length for an RNA agent described herein.

R⁷⁰ is H; or is together combined with R⁴⁰, R⁵⁰, or R⁶⁰ to form an [—O—CH₂—] covalently bound bridge between the sugar 2′ and 4′ carbons.

R⁸ is alkyl, cycloalkyl, aryl, aralkyl, heterocyclyl, heteroaryl, amino acid, or sugar; and R⁹ is NH₂, alkylamino, dialkylamino, heterocyclyl, arylamino, diaryl amino, heteroaryl amino, diheteroaryl amino, or amino acid. m is 0-1,000,000, n is 0-20, and g is 0-2.

Preferred nucleoside surrogates have the following structure (see Formula 4 below):

SLR¹⁰⁰-(M-SLR²⁰⁰)ₓ-M-SLR³⁰⁰ FORMULA 4

S is a nucleoside surrogate selected from the group consisting of mophilino, cyclobutyl, pyrrolidine and peptide nucleic acid. L is a linker and is selected from the group consisting of CH₂(CH₂)_{g}; N(CH₂)_{g}; O(CH₂)_{g}; S(CH₂)_{g}; —C(O)(CH₂)ₙ— or may be absent. M is an amide bond; sulfonamide; sulfinate; phosphate group; modified phosphate group as described herein; or may be absent.

R¹⁰⁰, R²⁰⁰, and R³⁰⁰ are each, independently, H (i.e., abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil substituted 1,2,4,-triazoles, 2-pyridinones, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N-6-isopentenyladenine, N-methylguanines, or O-alkylated bases.

x is 5-100, or chosen to comply with a length for an RNA agent described herein; and g is 0-2.

Enhanced nuclease resistance. An iRNA agent, e.g., an iRNA agent that targets ApoB can have enhanced resistance to nucleases. One way to increase resistance is to identify cleavage sites and modify such sites to inhibit cleavage. For example, the dinucleotides 5′-UA-3′,5′-UG-3′,5′-CA-3′,5′-UU-3′,5′-CC-3′,5′-CU-3′, or 5′-UC-3′ can serve as cleavage sites.

For increased nuclease resistance and/or binding affinity to the target, an iRNA agent, e.g., the sense and/or antisense strands of the iRNA agent, can include, for example, 2′-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Preferred substitutents are 2′-methoxyethyl, 2′-OCH3, 2′-O-allyl, 2′-C— allyl, and 2′-fluoro.

To maximize nuclease resistance, the 2′ modifications can be used in combination with one or more phosphate linker modifications (e.g., phosphorothioate). The so-called “chimeric” oligonucleotides are those that contain two or more different modifications.

In certain embodiments, all the pyrimidines of an iRNA agent carry a 2′-modification, and the iRNA agent therefore has enhanced resistance to endonucleases. Enhanced nuclease resistance can also be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; at least one 5′-cytidine-cytidine-3′ (5′-CC-3′) dinucleotide, wherein the 5′-cytidine is a 2′-modified nucleotide; at least one 5′-cytidine-uridine-3′ (5′-CU-3′) dinucleotide, wherein the 5′-cytidine is a 2′-modified nucleotide; or at least one 5′-uridine-cytidine-3′ (5′-UC-3′) dinucleotide, wherein the 5′-uridine is a 2′-modified nucleotide. The iRNA agent can include at least 2, at least 3, at least 4 or at least 5 of such dinucleotides.

The inclusion of furanose sugars in the oligonucleotide backbone can also decrease endonucleolytic cleavage. An iRNA agent can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′-end of oligonucleotide. Even small alkyl chains, aryl groups, or heterocyclic conjugates or modified sugars (D-ribose, deoxyribose, glucose etc.) can block 3′-5′-exonucleases.

Similarly, 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′-end of oligonucleotide. Even small alkyl chains, aryl groups, or heterocyclic conjugates or modified sugars (D-ribose, deoxyribose, glucose etc.) can block 3′-5′-exonucleases.

An iRNA agent can have increased resistance to nucleases when a duplexed iRNA agent includes a single-stranded nucleotide overhang on at least one end. In preferred embodiments, the nucleotide overhang includes 1 to 4, preferably 2 to 3, unpaired nucleotides. In a preferred embodiment, the unpaired nucleotide of the single-stranded overhang that is directly adjacent to the terminal nucleotide pair contains a purine base, and the terminal nucleotide pair is a G-C pair, or at least two of the last four complementary nucleotide pairs are G-C pairs. In further embodiments, the nucleotide overhang may have 1 or 2 unpaired nucleotides, and in an exemplary embodiment the nucleotide overhang is 5′-GC-3′. In preferred embodiments, the nucleotide overhang is on the 3′-end of the antisense strand. In one embodiment, the iRNA agent includes the motif 5′-CGC-3′ on the 3′-end of the antisense strand, such that a 2-nt overhang 5′-GC-3′ is formed.

An iRNA agent can include monomers which have been modified so as to inhibit degradation, e.g., by nucleases, e.g., endonucleases or exonucleases, found in the body of a subject. These monomers are referred to herein as NRMs, or nuclease resistance promoting monomers or modifications. In many cases these modifications will modulate other properties of the iRNA agent as well, e.g., the ability to interact with a protein, e.g., a transport protein, e.g., serum albumin, or a member of the RISC(RNA-induced Silencing Complex), or the ability of the first and second sequences to form a duplex with one another or to form a duplex with another sequence, e.g., a target molecule.

While not wishing to be bound by theory, it is believed that modifications of the sugar, base, and/or phosphate backbone in an iRNA agent can enhance endonuclease and exonuclease resistance, and can enhance interactions with transporter proteins and one or more of the functional components of the RISC complex. Preferred modifications are those that increase exonuclease and endonuclease resistance and thus prolong the half-life of the iRNA agent prior to interaction with the RISC complex, but at the same time do not render the iRNA agent resistant to endonuclease activity in the RISC complex. Again, while not wishing to be bound by any theory, it is believed that placement of the modifications at or near the 3′ and/or 5′ end of antisense strands can result in iRNA agents that meet the preferred nuclease resistance criteria delineated above. Again, still while not wishing to be bound by any theory, it is believed that placement of the modifications at e.g., the middle of a sense strand can result in iRNA agents that are relatively less likely to undergo off-targeting.

Modifications described herein can be incorporated into any double-stranded RNA and RNA-like molecule described herein, e.g., an iRNA agent. An iRNA agent may include a duplex comprising a hybridized sense and antisense strand, in which the antisense strand and/or the sense strand may include one or more of the modifications described herein. The anti sense strand may include modifications at the 3′ end and/or the 5′ end and/or at one or more positions that occur 1-6 (e.g., 1-5, 1-4, 1-3, 1-2) nucleotides from either end of the strand. The sense strand may include modifications at the 3′ end and/or the 5′ end and/or at any one of the intervening positions between the two ends of the strand. The iRNA agent may also include a duplex comprising two hybridized antisense strands. The first and/or the second antisense strand may include one or more of the modifications described herein. Thus, one and/or both antisense strands may include modifications at the 3′ end and/or the 5′ end and/or at one or more positions that occur 1-6 (e.g., 1-5, 1-4, 1-3, 1-2) nucleotides from either end of the strand. Particular configurations are discussed below.

Modifications that can be useful for producing iRNA agents that meet the preferred nuclease resistance criteria delineated above can include one or more of the following chemical and/or stereochemical modifications of the sugar, base, and/or phosphate backbone:

(i) chiral (S_{P}) thioates. Thus, preferred NRMs include nucleotide dimers with an enriched or pure for a particular chiral form of a modified phosphate group containing a heteroatom at the nonbridging position, e.g., Sp or Rp, at the position X, where this is the position normally occupied by the oxygen. The atom at X can also be S, Se, Nr₂, or Br₃. When X is S, enriched or chirally pure Sp linkage is preferred. Enriched means at least 70, 80, 90, 95, or 99% of the preferred form. Such NRMs are discussed in more detail below;

(ii) attachment of one or more cationic groups to the sugar, base, and/or the phosphorus atom of a phosphate or modified phosphate backbone moiety. Thus, preferred NRMs include monomers at the terminal position derivatized at a cationic group. As the 5′ end of an antisense sequence should have a terminal —OH or phosphate group this NRM is preferably not used at the 5′ end of an anti-sense sequence. The group should be attached at a position on the base which minimizes interference with H bond formation and hybridization, e.g., away form the face which interacts with the complementary base on the other strand, e.g, at the 5′ position of a pyrimidine or a 7-position of a purine. These are discussed in more detail below;

(iii) nonphosphate linkages at the termini Thus, preferred NRMs include Non-phosphate linkages, e.g., a linkage of 4 atoms which confers greater resistance to cleavage than does a phosphate bond. Examples include 3′ CH2-NCH₃—O—CH2-5′ and 3′ CH2-NH—(O═)—CH2-5′;

(iv) 3′-bridging thiophosphates and 5′-bridging thiophosphates. Thus, preferred NRM's can included these structures;

(v) L-RNA, 2′-5′ linkages, inverted linkages, a-nucleosides. Thus, other preferred NRM's include: L nucleosides and dimeric nucleotides derived from L-nucleosides; 2′-5′ phosphate, non-phosphate and modified phosphate linkages (e.g., thiophosphates, phosphoramidates and boronophosphates); dimers having inverted linkages, e.g., 3′-3′ or 5′-5′ linkages; monomers having an alpha linkage at the 1′ site on the sugar, e.g., the structures described herein having an alpha linkage;

(vi) conjugate groups. Thus, preferred NRM's can include e.g., a targeting moiety or a conjugated ligand described herein conjugated with the monomer, e.g., through the sugar, base, or backbone;

(vi) abasic linkages. Thus, preferred NRM's can include an abasic monomer, e.g., an abasic monomer as described herein (e.g., a nucleobaseless monomer); an aromatic or heterocyclic or polyheterocyclic aromatic monomer as described herein; and

(vii) 5′-phosphonates and 5′-phosphate prodrugs. Thus, preferred NRM's include monomers, preferably at the terminal position, e.g., the 5′ position, in which one or more atoms of the phosphate group is derivatized with a protecting group, which protecting group or groups, are removed as a result of the action of a component in the subject's body, e.g, a carboxyesterase or an enzyme present in the subject's body. E.g., a phosphate prodrug in which a carboxy esterase cleaves the protected molecule resulting in the production of a thioate anion which attacks a carbon adjacent to the O of a phosphate and resulting in the production of an unprotected phosphate.

One or more different NRM modifications can be introduced into an iRNA agent or into a sequence of an iRNA agent. An NRM modification can be used more than once in a sequence or in an iRNA agent. As some NRM's interfere with hybridization the total number incorporated, should be such that acceptable levels of iRNA agent duplex formation are maintained.

In some embodiments NRM modifications are introduced into the terminal the cleavage site or in the cleavage region of a sequence (a sense strand or sequence) which does not target a desired sequence or gene in the subject. This can reduce off-target silencing.

Chiral S_{P} Thioates

A modification can include the alteration, e.g., replacement, of one or both of the non-linking (X and Y) phosphate oxygens and/or of one or more of the linking (W and Z) phosphate oxygens. Formula X below depicts a phosphate moiety linking two sugar/sugar surrogate-base moieties, SB₁ and SB₂.

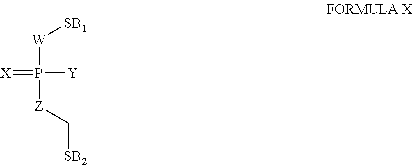

In certain embodiments, one of the non-linking phosphate oxygens in the phosphate backbone moiety (X and Y) can be replaced by any one of the following: S, Se, BR₃ (R is hydrogen, alkyl, aryl, etc.), C (i.e., an alkyl group, an aryl group, etc.), H, NR₂ (R is hydrogen, alkyl, aryl, etc.), or OR(R is alkyl or aryl). The phosphorus atom in an unmodified phosphate group is achiral. However, replacement of one of the non-linking oxygens with one of the above atoms or groups of atoms renders the phosphorus atom chiral; in other words a phosphorus atom in a phosphate group modified in this way is a stereogenic center. The stereogenic phosphorus atom can possess either the “R” configuration (herein R_{P}) or the “S” configuration (herein S_{P}). Thus if 60% of a population of stereogenic phosphorus atoms have the R_{P} configuration, then the remaining 40% of the population of stereogenic phosphorus atoms have the S_{P} configuration.

In some embodiments, iRNA agents, having phosphate groups in which a phosphate non-linking oxygen has been replaced by another atom or group of atoms, may contain a population of stereogenic phosphorus atoms in which at least about 50% of these atoms (e.g., at least about 60% of these atoms, at least about 70% of these atoms, at least about 80% of these atoms, at least about 90% of these atoms, at least about 95% of these atoms, at least about 98% of these atoms, at least about 99% of these atoms) have the Sp configuration. Alternatively, iRNA agents having phosphate groups in which a phosphate non-linking oxygen has been replaced by another atom or group of atoms may contain a population of stereogenic phosphorus atoms in which at least about 50% of these atoms (e.g., at least about 60% of these atoms, at least about 70% of these atoms, at least about 80% of these atoms, at least about 90% of these atoms, at least about 95% of these atoms, at least about 98% of these atoms, at least about 99% of these atoms) have the R_{P} configuration. In other embodiments, the population of stereogenic phosphorus atoms may have the S_{P} configuration and may be substantially free of stereogenic phosphorus atoms having the R^{P} configuration. In still other embodiments, the population of stereogenic phosphorus atoms may have the R_{P} configuration and may be substantially free of stereogenic phosphorus atoms having the S_{P} configuration. As used herein, the phrase “substantially free of stereogenic phosphorus atoms having the R_{P} configuration” means that moieties containing stereogenic phosphorus atoms having the R_{P} configuration cannot be detected by conventional methods known in the art (chiral HPLC, ¹H NMR analysis using chiral shift reagents, etc.). As used herein, the phrase “substantially free of stereogenic phosphorus atoms having the S_{P} configuration” means that moieties containing stereogenic phosphorus atoms having the S_{P} configuration cannot be detected by conventional methods known in the art (chiral HPLC, ¹H NMR analysis using chiral shift reagents, etc.).

In a preferred embodiment, modified iRNA agents contain a phosphorothioate group, i.e., a phosphate groups in which a phosphate non-linking oxygen has been replaced by a sulfur atom. In an especially preferred embodiment, the population of phosphorothioate stereogenic phosphorus atoms may have the S_{P} configuration and be substantially free of stereogenic phosphorus atoms having the R^{P} configuration.

Phosphorothioates may be incorporated into iRNA agents using dimers e.g., formulas X-1 and X-2. The former can be used to introduce phosphorothioate

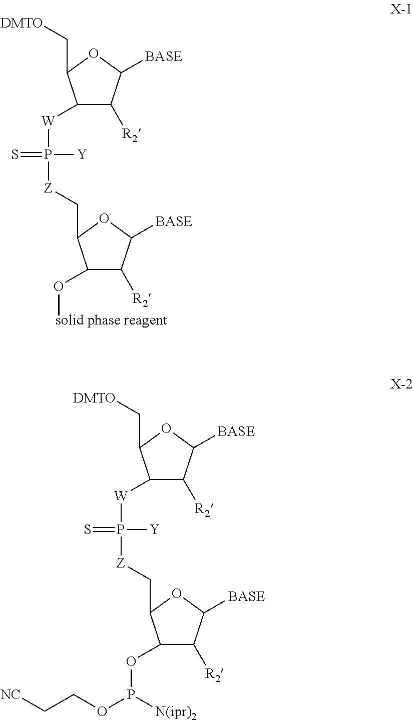

at the 3′ end of a strand, while the latter can be used to introduce this modification at the 5′ end or at a position that occurs e.g., 1, 2, 3, 4, 5, or 6 nucleotides from either end of the strand. In the above formulas, Y can be 2-cyanoethoxy, W and Z can be O, R_{2′} can be, e.g., a substituent that can impart the C-3 endo configuration to the sugar (e.g., OH, F, OCH₃), DMT is dimethoxytrityl, and “BASE” can be a natural, unusual, or a universal base.

X-1 and X-2 can be prepared using chiral reagents or directing groups that can result in phosphorothioate-containing dimers having a population of stereogenic phosphorus atoms having essentially only the R_{P} configuration (i.e., being substantially free of the S_{P} configuration) or only the S_{P} configuration (i.e., being substantially free of the R_{P} configuration). Alternatively, dimers can be prepared having a population of stereogenic phosphorus atoms in which about 50% of the atoms have the R_{P} configuration and about 50% of the atoms have the S_{P} configuration. Dimers having stereogenic phosphorus atoms with the R_{P} configuration can be identified and separated from dimers having stereogenic phosphorus atoms with the S_{P} configuration using e.g., enzymatic degradation and/or conventional chromatography techniques.

### Cationic Groups

Modifications can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); or NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino)

### Nonphosphate Linkages

Modifications can also include the incorporation of nonphosphate linkages at the 5′ and/or 3′ end of a strand. Examples of nonphosphate linkages which can replace the phosphate group include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methyl phosphonate and hydroxylamino groups.

3′-bridging thiophosphates and 5′-bridging thiophosphates; locked-RNA, 2′-5′ linkages, inverted linkages, α-nucleosides; conjugate groups; abasic linkages; and 5′-phosphonates and 5′-phosphate prodrugs: Referring to formula X above, modifications can include replacement of one of the bridging or linking phosphate oxygens in the phosphate backbone moiety (W and Z). Unlike the situation where only one of X or Y is altered, the phosphorus center in the phosphorodithioates is achiral which precludes the formation of iRNA agents containing a stereogenic phosphorus atom.

Modifications can also include linking two sugars via a phosphate or modified phosphate group through the 2′ position of a first sugar and the 5′ position of a second sugar. Also contemplated are inverted linkages in which both a first and second sugar are eached linked through the respective3′ positions. Modified RNA's can also include “abasic” sugars, which lack a nucleobase at C-1′. The sugar group can also contain one or more carbons that possess the opposite stereochemical configuration than that of the corresponding carbon in ribose. Thus, a modified iRNA agent can include nucleotides containing e.g., arabinose, as the sugar. In another subset of this modification, the natural, unusual, or universal base may have the α-configuration. Modifcations can also include L-RNA.

Modifications can also include 5′-phosphonates, e.g., P(O)(O⁻)₂—X—C^{5′}-sugar (X=CH2, CF2, CHF and 5′-phosphate prodrugs, e.g., P(O)[OCH2CH2SC(O)R]₂CH₂C^{5′}-sugar. In the latter case, the prodrug groups may be decomposed via reaction first with carboxy esterases. The remaining ethyl thiolate group via intramolecular S_{N}2 displacement can depart as episulfide to afford the underivatized phosphate group.

Modification can also include the addition of conjugating groups described elseqhere herein, which are prefereably attached to an iRNA agent through any amino group available for conjugation.

Nuclease resistant modifications include some which can be placed only at the terminus and others which can go at any position. Generally the modifications that can inhibit hybridization so it is preferably to use them only in terminal regions, and preferrable to not use them at the cleavage site or in the cleavage region of an sequence which targets a subject sequence or gene. The can be used anywhere in a sense sequence, provided that sufficient hybridization between the two sequences of the iRNA agent is maintained. In some embodiments it is desirabable to put the NRM at the cleavage site or in the cleavage region of a sequence which does not target a subject sequence or gene, as it can minimize off-target silencing.

In addition, an iRNA agent described herein can have an overhang which does not form a duplex structure with the other sequence of the iRNA agent—it is an overhang, but it does hybridize, either with itself, or with another nucleic acid, other than the other sequence of the iRNA agent.

In most cases, the nuclease-resistance promoting modifications will be distributed differently depending on whether the sequence will target a sequence in the subject (often referred to as an anti-sense sequence) or will not target a sequence in the subject (often referred to as a sense sequence). If a sequence is to target a sequence in the subject, modifications which interfer with or inhibit endonuclease cleavage should not be inserted in the region which is subject to RISC mediated cleavage, e.g., the cleavage site or the cleavage region (As described in Elbashir et al., 2001, Genes and Dev. 15: 188, hereby incorporated by reference, cleavage of the target occurs about in the middle of a 20 or 21 nt guide RNA, or about 10 or 11 nucleotides upstream of the first nucleotide which is complementary to the guide sequence. As used herein cleavage site refers to the nucleotide on either side of the cleavage site, on the target or on the iRNA agent strand which hybridizes to it. Cleavage region means an nucleotide with 1, 2, or 3 nucletides of the cleave site, in either direction.)

Such modifications can be introduced into the terminal regions, e.g., at the terminal position or with 2, 3, 4, or 5 positions of the terminus, of a sequence which targets or a sequence which does not target a sequence in the subject.

An iRNA agent can have a first and a second strand chosen from the following:

a first strand which does not target a sequence and which has an NRM modification at or within 1, 2, 3, 4, 5, or 6 positions from the 3′ end;

a first strand which does not target a sequence and which has an NRM modification at or within 1, 2, 3, 4, 5, or 6 positions from the 5′ end;

a first strand which does not target a sequence and which has an NRM modification at or within 1, 2, 3, 4, 5, or 6 positions from the 3′ end and which has a NRM modification at or within 1, 2, 3, 4, 5, or 6 positions from the 5′ end;

a first strand which does not target a sequence and which has an NRM modification at the cleavage site or in the cleavage region;

a first strand which does not target a sequence and which has an NRM modification at the cleavage site or in the cleavage region and one or more of an NRM modification at or within 1, 2, 3, 4, 5, or 6 positions from the 3′ end, a NRM modification at or within 1, 2, 3, 4, 5, or 6 positions from the 5′ end, or NRM modifications at or within 1, 2, 3, 4, 5, or 6 positions from both the 3′ and the 5′ end; and

a second strand which targets a sequence and which has an NRM modification at or within 1, 2, 3, 4, 5, or 6 positions from the 3′ end;

a second strand which targets a sequence and which has an NRM modification at or within 1, 2, 3, 4, 5, or 6 positions from the 5′ end (5′ end NRM modifications are preferentially not at the terminus but rather at a position 1, 2, 3, 4, 5, or 6 away from the 5′ terminus of an antisense strand);

a second strand which targets a sequence and which has an NRM modification at or within 1, 2, 3, 4, 5, or 6 positions from the 3′ end and which has a NRM modification at or within 1, 2, 3, 4, 5, or 6 positions from the 5′ end;

a second strand which targets a sequence and which preferably does not have an an NRM modification at the cleavage site or in the cleavage region;

a second strand which targets a sequence and which does not have an NRM modification at the cleavage site or in the cleavage region and one or more of an NRM modification at or within 1, 2, 3, 4, 5, or 6 positions from the 3′ end, a NRM modification at or within 1, 2, 3, 4, 5, or 6 positions from the 5′ end, or NRM modifications at or within 1, 2, 3, 4, 5, or 6 positions from both the 3′ and the 5′ end(5′ end NRM modifications are preferentially not at the terminus but rather at a position 1, 2, 3, 4, 5, or 6 away from the 5′ terminus of an antisense strand).

An iRNA agent can also target two sequences and can have a first and second strand chosen from:

a first strand which targets a sequence and which has an NRM modification at or within 1, 2, 3, 4, 5, or 6 positions from the 3′ end;

a first strand which targets a sequence and which has an NRM modification at or within 1, 2, 3, 4, 5, or 6 positions from the 5′ end (5′ end NRM modifications are preferentially not at the terminus but rather at a position 1, 2, 3, 4, 5, or 6 away from the 5′ terminus of an antisense strand);

a first strand which targets a sequence and which has an NRM modification at or within 1, 2, 3, 4, 5, or 6 positions from the 3′ end and which has a NRM modification at or within 1, 2, 3, 4, 5, or 6 positions from the 5′ end;

a first strand which targets a sequence and which preferably does not have an NRM modification at the cleavage site or in the cleavage region;

a first strand which targets a sequence and which dose not have an NRM modification at the cleavage site or in the cleavage region and one or more of an NRM modification at or within 1, 2, 3, 4, 5, or 6 positions from the 3′ end, a NRM modification at or within 1, 2, 3, 4, 5, or 6 positions from the 5′ end, or NRM modifications at or within 1, 2, 3, 4, 5, or 6 positions from both the 3′ and the 5′ end(5′ end NRM modifications are preferentially not at the terminus but rather at a position 1, 2, 3, 4, 5, or 6 away from the 5′ terminus of an antisense strand) and

a second strand which targets a sequence and which has an NRM modification at or within 1, 2, 3, 4, 5, or 6 positions from the 3′ end;

a second strand which targets a sequence and which has an NRM modification at or within 1, 2, 3, 4, 5, or 6 positions from the 5′ end (5′ end NRM modifications are preferentially not at the terminus but rather at a position 1, 2, 3, 4, 5, or 6 away from the 5′ terminus of an antisense strand);

a second strand which targets a sequence and which has an NRM modification at or within 1, 2, 3, 4, 5, or 6 positions from the 3′ end and which has a NRM modification at or within 1, 2, 3, 4, 5, or 6 positions from the 5′ end;

a second strand which targets a sequence and which preferably does not have an an NRM modification at the cleavage site or in the cleavage region;

a second strand which targets a sequence and which dose not have an NRM modification at the cleavage site or in the cleavage region and one or more of an NRM modification at or within 1, 2, 3, 4, 5, or 6 positions from the 3′ end, a NRM modification at or within 1, 2, 3, 4, 5, or 6 positions from the 5′ end, or NRM modifications at or within 1, 2, 3, 4, 5, or 6 positions from both the 3′ and the 5′ end(5′ end NRM modifications are preferentially not at the terminus but rather at a position 1, 2, 3, 4, 5, or 6 away from the 5′ terminus of an antisense strand).

Ribose Mimics: The monomers and methods described herein can be used to prepare an RNA, e.g., an iRNA agent, that incorporates a ribose mimic, such as those described herein and those described in copending co-owned U.S. Provisional Application Ser. No. 60/454,962, filed on Mar. 13, 2003, and International Application No. PCT/US04/07070, both of which are hereby incorporated by reference.

Thus, an aspect of the invention features an iRNA agent that includes a secondary hydroxyl group, which can increase efficacy and/or confer nuclease resistance to the agent. Nucleases, e.g., cellular nucleases, can hydrolyze nucleic acid phosphodiester bonds, resulting in partial or complete degradation of the nucleic acid. The secondary hydroxy group confers nuclease resistance to an iRNA agent by rendering the iRNA agent less prone to nuclease degradation relative to an iRNA which lacks the modification. While not wishing to be bound by theory, it is believed that the presence of a secondary hydroxyl group on the iRNA agent can act as a structural mimic of a 3′ ribose hydroxyl group, thereby causing it to be less susceptible to degradation.

The secondary hydroxyl group refers to an “OH” radical that is attached to a carbon atom substituted by two other carbons and a hydrogen. The secondary hydroxyl group that confers nuclease resistance as described above can be part of any acyclic carbon-containing group. The hydroxyl may also be part of any cyclic carbon-containing group, and preferably one or more of the following conditions is met (1) there is no ribose moiety between the hydroxyl group and the terminal phosphate group or (2) the hydroxyl group is not on a sugar moiety which is coupled to a base. The hydroxyl group is located at least two bonds (e.g., at least three bonds away, at least four bonds away, at least five bonds away, at least six bonds away, at least seven bonds away, at least eight bonds away, at least nine bonds away, at least ten bonds away, etc.) from the terminal phosphate group phosphorus of the iRNA agent. In preferred embodiments, there are five intervening bonds between the terminal phosphate group phosphorus and the secondary hydroxyl group.

Preferred iRNA agent delivery modules with five intervening bonds between the terminal phosphate group phosphorus and the secondary hydroxyl group have the following structure (see formula Y below):

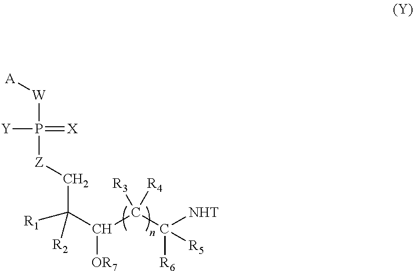

Referring to formula Y, A is an iRNA agent, including any iRNA agent described herein. The iRNA agent may be connected directly or indirectly (e.g., through a spacer or linker) to “W” of the phosphate group. These spacers or linkers can include e.g., —(CH₂)ₙ—, —(CH₂)ₙN—, —(CH₂)ₙO —, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents.

The iRNA agents can have a terminal phosphate group that is unmodified (e.g., W, X, Y, and Z are O) or modified. In a modified phosphate group, W and Z can be independently NH, O, or S; and X and Y can be independently S, Se, BH₃⁻, C₁-C₆ alkyl, C₆-C₁₀ aryl, H, O, O⁻, alkoxy or amino (including alkylamino, arylamino, etc.). Preferably, W, X and Z are O and Y is S.

R₁ and R₃ are each, independently, hydrogen; or C₁-C₁₀₀ alkyl, optionally substituted with hydroxyl, amino, halo, phosphate or sulfate and/or may be optionally inserted with N, O, S, alkenyl or alkynyl.

R₂ is hydrogen; C₁-C₁₀₀ alkyl, optionally substituted with hydroxyl, amino, halo, phosphate or sulfate and/or may be optionally inserted with N, O, S, alkenyl or alkynyl; or, when n is 1, R₂ may be taken together with R₄ or R₆ to form a ring of 5-12 atoms.

R₄ is hydrogen; C₁-C₁₀₀ alkyl, optionally substituted with hydroxyl, amino, halo, phosphate or sulfate and/or may be optionally inserted with N, O, S, alkenyl or alkynyl; or, when n is 1, R₄ may be taken together with R₂ or R₅ to form a ring of 5-12 atoms.

R₅ is hydrogen, C₁-C₁₀₀ alkyl optionally substituted with hydroxyl, amino, halo, phosphate or sulfate and/or may be optionally inserted with N, O, S, alkenyl or alkynyl; or, when n is 1, R₅ may be taken together with R₄ to form a ring of 5-12 atoms.

R₆ is hydrogen, C₁-C₁₀₀ alkyl, optionally substituted with hydroxyl, amino, halo, phosphate or sulfate and/or may be optionally inserted with N, O, S, alkenyl or alkynyl, or, when n is 1, R₆ may be taken together with R₂ to form a ring of 6-10 atoms;

R₇ is hydrogen, C₁-C₁₀₀ alkyl, or C(O)(CH₂)_{q}C(O)NHR₉; T is hydrogen or a functional group; n and q are each independently 1-100; R₈ is C₁-C₁₀ alkyl or C₆-C₁₀ aryl; and R₉ is hydrogen, C1-C10 alkyl, C6-C10 aryl or a solid support agent.

Preferred embodiments may include one of more of the following subsets of iRNA agent delivery modules.

In one subset of RNAi agent delivery modules, A can be connected directly or indirectly through a terminal 3′ or 5′ ribose sugar carbon of the RNA agent.

In another subset of RNAi agent delivery modules, X, W, and Z are O and Y is S.

In still yet another subset of RNAi agent delivery modules, n is 1, and R₂ and R₆ are taken together to form a ring containing six atoms and R₄ and R₅ are taken together to form a ring containing six atoms. Preferably, the ring system is a trans-decalin. For example, the RNAi agent delivery module of this subset can include a compound of Formula (Y-1):

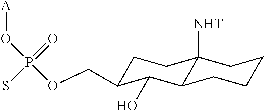

The functional group can be, for example, a targeting group (e.g., a steroid or a carbohydrate), a reporter group (e.g., a fluorophore), or a label (an isotopically labelled moiety). The targeting group can further include protein binding agents, endothelial cell targeting groups (e.g., RGD peptides and mimetics), cancer cell targeting groups (e.g., folate Vitamin B12, Biotin), bone cell targeting groups (e.g., bisphosphonates, polyglutamates, polyaspartates), multivalent mannose (for e.g., macrophage testing), lactose, galactose, N-acetyl-galactosamine, monoclonal antibodies, glycoproteins, lectins, melanotropin, or thyrotropin.

As can be appreciated by the skilled artisan, methods of synthesizing the compounds of the formulae herein will be evident to those of ordinary skill in the art. The synthesized compounds can be separated from a reaction mixture and further purified by a method such as column chromatography, high pressure liquid chromatography, or recrystallization. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Pharmaceutical compositions. In one embodiment, the invention relates to a pharmaceutical composition containing a modified iRNA agent, as described in the preceding sections, and a pharmaceutically acceptable carrier, as described below. A pharmaceutical composition including the modified iRNA agent is useful for treating a disease caused by expression of a target gene. In this aspect of the invention, the iRNA agent of the invention is formulated as described below. The pharmaceutical composition is administered in a dosage sufficient to inhibit expression of the target gene.

The pharmaceutical compositions of the present invention are administered in dosages sufficient to inhibit the expression or activity of the target gene. Compositions containing the iRNA agent of the invention can be administered at surprisingly low dosages. A maximum dosage of 5 mg iRNA agent per kilogram body weight per day may be sufficient to inhibit or completely suppress the expression or activity of the target gene.

In general, a suitable dose of modified iRNA agent will be in the range of 0.001 to 500 milligrams per kilogram body weight of the recipient per day (e.g., about 1 microgram per kilogram to about 500 milligrams per kilogram, about 100 micrograms per kilogram to about 100 milligrams per kilogram, about 1 milligrams per kilogram to about 75 milligrams per kilogram, about 10 micrograms per kilogram to about 50 milligrams per kilogram, or about 1 microgram per kilogram to about 50 micrograms per kilogram). The pharmaceutical composition may be administered once per day, or the iRNA agent may be administered as two, three, four, five, six or more sub-doses at appropriate intervals throughout the day. In that case, the iRNA agent contained in each sub-dose must be correspondingly smaller in order to achieve the total daily dosage. The dosage unit can also be compounded for delivery over several days, e.g., using a conventional sustained release formulation which provides sustained release of the iRNA agent over a several day period. Sustained release formulations are well known in the art. In this embodiment, the dosage unit contains a corresponding multiple of the daily dose.

The skilled artisan will appreciate that certain factors may influence the dosage and timing required to effectively treat a subject, including but not limited to the severity of the infection or disease, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual iRNA agent encompassed by the invention can be made using conventional methodologies or on the basis of in vivo testing using an appropriate animal model, as described elsewhere herein.

Advances in mouse genetics have generated a number of mouse models for the study of various human diseases. For example, mouse repositories can be found at The Jackson Laboratory, Charles River Laboratories, Taconic, Harlan, Mutant Mouse Regional Resource Centers (MMRRC) National Network and at the European Mouse Mutant Archive. Such models may be used for in vivo testing of iRNA agent, as well as for determining a therapeutically effective dose.

The pharmaceutical compositions encompassed by the invention may be administered by any means known in the art including, but not limited to oral or parenteral routes, including intravenous, intramuscular, intraperitoneal, subcutaneous, transdermal, airway (aerosol), ocular, rectal, vaginal and topical (including buccal and sublingual) administration. In preferred embodiments, the pharmaceutical compositions are administered by intravenous or intraparenteral infusion or injection. The pharmaceutical compositions can also be administered intraparenchymally, intrathecally, and/or by stereotactic injection.

For oral administration, the iRNA agent useful in the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredient is mixed with water or an oil such as peanut oil, liquid paraffin or olive oil.

For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of iRNA agent in the cells that harbor the target gene or virus. Such substances include, for example, micellar structures, such as liposomes or capsids, as described below. Although microinjection, lipofection, viruses, viroids, capsids, capsoids, or other auxiliary agents are required to introduce iRNA agent into cell cultures, surprisingly these methods and agents are not necessary for uptake of iRNA agent in vivo. The iRNA agent of the present invention are particularly advantageous in that they do not require the use of an auxiliary agent to mediate uptake of the iRNA agent into the cell, many of which agents are toxic or associated with deleterious side effects. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions can also include encapsulated formulations to protect the iRNA agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

Toxicity and therapeutic efficacy of iRNA agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. iRNA agents that exhibit high therapeutic indices are preferred.

The data obtained from cell culture assays and animal studies can be used in formulating a range of dosage for use in humans. The dosages of compositions of the invention are preferably within a range of circulating concentrations that include the ED50 with little or no toxicity. The dosage may vary within this range depending upon the dosage form employed and the route of administration utilized. For any iRNA agent used in the method of the invention, the therapeutically effective dose can be estimated initially from cell culture assays. A dose may be formulated in animal models to achieve a circulating plasma concentration range of the iRNA agent or, when appropriate, of the polypeptide product of a target sequence (e.g., achieving a decreased concentration of the polypeptide) that includes the IC50 (i.e., the concentration of the test iRNA agent which achieves a half-maximal inhibition of symptoms) as determined in cell culture. Such information can be used to more accurately determine useful doses in humans Levels in plasma may be measured, for example, by high performance liquid chromatography.

In addition to their administration individually or as a plurality, as discussed above, iRNA agents relating to the invention can be administered in combination with other known agents effective in treating viral infections and diseases. In any event, the administering physician can adjust the amount and timing of iRNA agent administration on the basis of results observed using standard measures of efficacy known in the art or described herein.

For oral administration, the iRNA agent useful in the invention will generally be provided in the form of tablets or capsules, as a powder or granules, or as an aqueous solution or suspension.

The pharmaceutical compositions encompassed by the invention may be administered by any means known in the art including, but not limited to, oral or parenteral routes, including intravenous, intramuscular, intraperitoneal, subcutaneous, transdermal, airway (aerosol), ocular, rectal, vaginal, and topical (including buccal and sublingual) administration. In preferred embodiments, the pharmaceutical compositions are administered by intravenous or intraparenteral infusion or injection. The pharmaceutical compositions can also be administered intraparenchymally, intrathecally, and/or by stereotactic injection.

Methods for identifying iRNA agents having increased stability. In yet another aspect, the invention relates to methods for identifying iRNA agent having increased stability in biological tissues and fluids such as serum. iRNA agent having increased stability have enhanced resistance to degradation, e.g., by chemicals or nucleases (particularly endonucleases) which normally degrade RNA molecules. Methods for detecting increases in nucleic acid stability are well known in the art. Any assay capable of measuring or detecting differences between a test iRNA agent and a control iRNA agent in any measurable physical parameter may be suitable for use in the methods of the present invention. In general, because the inhibitory effect of an iRNA agent on a target gene activity or expression requires that the molecule remain intact, the stability of a particular iRNA agent can be evaluated indirectly by observing or measuring a property associated with the expression of the gene. Thus, the relative stability of an iRNA agent can be determined by observing or detecting (1) an absence or observable decrease in the level of the protein encoded by the target gene, (2) an absence or observable decrease in the level of mRNA product from the target gene, and (3) a change or loss in phenotype associated with expression of the target gene. In the context of a medical treatment, the stability of an iRNA agent may be evaluated based on the degree of the inhibition of expression or function of the target gene, which in turn may be assessed based on a change in the disease condition of the patient, such as reduction in symptoms, remission, or a change in disease state.

In one embodiment, the method includes preparing an iRNA agent as described above (e.g., through chemical synthesis), incubating the iRNA agent with a biological sample, then analyzing and identifying those iRNA agent that exhibit an increased stability as compared to a control iRNA agent.

In an exemplified embodiment, iRNA agent is produced in vitro by mixing/annealing complementary single-stranded RNA strands, preferably in a molar ratio of at least about 3:7, more preferably in a molar ratio of about 4:6, and most preferably in essentially equal molar amounts (e.g., a molar ratio of about 5:5). Preferably, the single-stranded RNA strands are denatured prior to mixing/annealing, and the buffer in which the mixing/annealing reaction takes place contains a salt, preferably potassium chloride. Single-stranded RNA strands may be synthesized by solid phase synthesis using, for example, an Expedite 8909 synthesizer (Applied Biosystems, Applera Deutschland GmbH, Darmstadt, Germany), as described above.

iRNA agent are incubated with a biological sample under the conditions sufficient or optimal for enzymatic function. After incubating with a biological sample, the stability of the iRNA agent is analyzed by means conventional in the art, for example using RNA gel electrophoresis as exemplified herein. For example, when the sample is serum, the iRNA agent may be incubated at a concentration of 1-10 μM, preferably 2-8 μM, more preferably 3-6 μM, and most preferably 4-5 μM. The incubation temperature is preferably between 25° C. and 45° C., more preferably between 35° C. and 40° C., and most preferably about 37° C.

The biological sample used in the incubation step may be derived from tissues, cells, biological fluids or isolates thereof. For example, the biological sample may be isolated from a subject, such as a whole organism or a subset of its tissues or cells. The biological sample may also be a component part of the subject, such as a body fluid, including but not limited to blood, serum, plasma, mucus, lymphatic fluid, synovial fluid, cerebrospinal fluid, saliva, amniotic fluid, amniotic cord blood, urine, vaginal fluid and semen. Preferably, the biological sample is a serum derived from a blood sample of a subject. The subject is preferably a mammal, more preferably a human or a mouse.

In another embodiment, the method includes selecting an iRNA agent having increased stability by measuring the mRNA and/or protein expression levels of a target gene in a cell following introduction of the iRNA agent. In this embodiment, an iRNA agent of the invention inhibits expression of a target gene in a cell, and thus the method includes selecting an iRNA agent that induces a measurable reduction in expression of a target gene as compared to a control iRNA agent. Assays that measure gene expression by monitoring RNA and/or protein levels can be performed within about 24 hours following uptake of the iRNA agent by the cell. For example, RNA levels can be measured by Northern blot techniques, RNAse Protection Assays, or Quality Control-PCR (QC-PCR) (including quantitative reverse transcription coupled PCR (RT-PCR)) and analogous methods known in the art. Protein levels can be assayed, for example, by Western blot techniques, flow cytometry, or reporter gene expression (e.g., expression of a fluorescent reporter protein, such as green fluorescent protein (GFP)). RNA and/or protein levels resulting from target gene expression can be measured at regular time intervals following introduction of the test iRNA agent, and the levels are compared to those following introduction of a control iRNA agent into cells. A control iRNA agent can be a nonsensical iRNA agent (i.e., an iRNA agent having a scrambled sequence that does not target any nucleotide sequence in the subject), an iRNA agent that can target a gene not present in the subject (e.g., a luciferase gene, when the iRNA agent is tested in human cells), or an iRNA agent otherwise previously shown to be ineffective at silencing the target gene. The mRNA and protein levels of the test sample and the control sample can be compared. The test iRNA agent is selected as having increased stability when there is a measurable reduction in expression levels following absorption of the test iRNA agent as compared to the control iRNA agent. mRNA and protein measurements can be made using any art-recognized technique (see, e.g., Chiang, M. Y., et al., J. Biol. Chem. (1991) 266:18162-71; Fisher, T, et al., Nucl. Acids Res. (1993) 21:3857; and Chen et al., J. Biol. Chem. (1996) 271:28259).

Methods for identifying iRNA agents with ability to inhibit gene expression. The ability of an iRNA agent composition of the invention to inhibit gene expression can be measured using a variety of techniques known in the art. For example, Northern blot analysis can be used to measure the presence of RNA encoding a target protein. The level of the specific mRNA produced by the target gene can be measured, e.g., using RT-PCR. Because iRNA agent directs the sequence-specific degradation of endogenous mRNA through RNAi, the selection methods of the invention encompass any technique that is capable of detecting a measurable reduction in the target RNA. In yet another example, Western blots can be used to measure the amount of target protein present. In still another embodiment, a phenotype influenced by the amount of the protein can be detected. Techniques for performing Western blots are well known in the art (see, e.g., Chen, et al., J. Biol. Chem. (1996) 271:28259).

When the target gene is to be silenced by an iRNA agent that targets a promoter sequence of the target gene, the target gene can be fused to a reporter gene, and reporter gene expression (e.g., transcription and/or translation) can be monitored. Similarly, when the target gene is to be silenced by an iRNA agent that targets a sequence other than a promoter, a portion of the target gene (e.g., a portion including the target sequence) can be fused with a reporter gene so that the reporter gene is transcribed. By monitoring a change in the expression of the reporter gene in the presence of the iRNA agent, it is possible to determine the effectiveness of the iRNA agent in inhibiting the expression of the reporter gene. The expression levels of the reporter gene in the presence of the test iRNA agent versus a control iRNA agent are then compared. The test iRNA agent is selected as having increased stability when there is a measurable reduction in expression levels of the reporter gene as compared to the control iRNA agent. Examples of reporter genes useful for use in the present invention include, without limitation, those coding for luciferase, GFP, chloramphenicol acetyl transferase (CAT), β-galactosidase, and alkaline phosphatase. Suitable reporter genes are described, for example, in Current Protocols in Molecular Biology, John Wiley & Sons, New York (Ausubel, F. A., et al., eds., 1989); Gould, S. J., and S. Subramani, Anal. Biochem. (1988) 7:404-408; Gorman, C. M., et al., Mol. Cell. Biol. (1982) 2:1044-1051; and Selden, R., et al., Mol. Cell. Biol. (1986) 6:3173-3179; each of which is hereby incorporated by reference.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Methods for identifying iRNA agents with ability to inhibit or stimulate the immune system. Modulation of the immune system can be measured for example by (i) measurement of either the mRNA or protein expression levels of a component (e.g., a growth factor, cytokine, or interleukin) of the immune system, e.g., in a cell or in an animal, (ii) measurement of the mRNA or protein levels of a protein factor activated by a component of the immune system (for example, NFKB), e.g., in a cell or in an animal, (iii) measurement of cell proliferation, e.g., in a tissue explant or a tissue of an animal

Evaluation of a candidate immunoselective iRNA agent can include incubating the modified strand (with or without its complement, but preferably annealed to its complement) with a biological system, e.g., a sample (e.g, a cell culture). The biological sample can be capable of expressing a component of the immune system. This allows identification of an iRNA agent that has an effect on the component. In one embodiment, the step of evaluating whether the iRNA agent modulates, e.g, stimulates or inhibits, an immune response includes evaluating expression of one or more growth factors, such as a cytokine or interleukin, or cell surface receptor protein, in a cell free, cell-based, or animal assay. Protein levels can be assayed, for example, by Western blot techniques, flow cytometry, or reporter gene expression (e.g., expression of a fluorescent reporter protein, such as green fluorescent protein (GFP)). The levels of mRNA of the protein of interest can be measured by Northern blot techniques, RNAse Protection Assays, or Quality Control-PCR (QC-PCR) (including quantitative reverse transcription coupled PCR (RT-PCR)) and analogous methods known in the art. RNA and/or protein levels resulting from target gene expression can be measured at regular time intervals following introduction of the test iRNA agent, and the levels are compared to those following introduction of a control iRNA agent into cells.

In one embodiment, the step of testing whether the modified iRNA agent modulates, e.g., stimulates, an immune response includes assaying for an interaction between the iRNA agent and a protein component of the immune system, e.g., a growth factor, such as a cytokine or interleukin, or a cell surface receptor protein. Exemplary assay methods include coimmunoprecipitation assays, bead-based co-isolation methods, nucleic acid footprint assays and colocalization experiments such as those facilitated by immunocytochemistry techniques.

Cell proliferaton can be monitored by following the uptake of [³H]thymidine or of a fluorescent dye. Cells were plated in a 96-well tissue culture plate and then incubated with the iRNA agent. For radiometric analysis, [³H]thymidine is added and incubation is continued. The cells are subsequently processed on a multichannel automated cell harvester (Cambridge Technology, Cambridge, Mass.) and counted in a liquid scintillation beta counter (Beckman Coulter). For fluorescence-based analysis, a commercially available assay, like the LIVE/DEAD Viability/Cytotoxicity assay from Molecular Probes can be used. The kit identifies live versus dead cells on the basis of membrane integrity and esterase activity. This kit can be used in microscopy, flow cytometry or microplate assays.

### EXAMPLES

### Example 1

### siRNA Modifications Enhanced Duplex Stability

Radiolabel method for monitoring serum stability of siRNA duplexes: siRNA duplexes were prepared at a stock concentration of 1 μM in which either the sense (S) or antisense strand (AS) contained a trace amount of 5′-³²P labeled material (e.g. ³²P—S/AS and S/³²P-AS). The presence of the end-labeled sense or antisense strand allowed for monitoring of the individual strand within the context of the siRNA duplex. Therefore, two duplex preparations were made for each siRNA sequence tested. siRNA duplexes were incubated in 90% human serum at a final concentration of 100 nM duplex. Samples were removed and quenched in a stop mix at appropriate times. For a typical time course, 10 seconds, 15 minutes, 30 minutes, 1 hour, 2 hours and 4 hours time points were taken. Samples were analyzed by denaturing polyacrylamide gel electrophoresis along with a control sample (4 hour buffer-alone incubation) and a partial alkaline hydrolysis ladder of the labeled sense or antisense strand as a marker. The gel was imaged using a Fuji phosphorimager to detect the full length sense and antisense strands along with any degradation fragments that were generated by serum nucleases during incubation.

Since there is the possibility of losing the 5′ phosphate label due to phosphatase activity in the serum, an alternative to 5′ end labeling is to place an internal ³²P or ³³P label within either the sense or antisense strand. This labeling method is much more laborious than 5′ end labeling and currently we have no evidence that dephosphorylation occurs during serum incubation.

A series of chemical modifications that fall into the following categories; backbone modification, sugar modification, nucleobase modification and 3′ conjugate, were tested and showed enhanced serum stability as compared to a unmodified siRNA duplex. A description of each modification, its location within the siRNA duplex, and the serum stability data follows.

Serum stability of unmodified parent duplex: The unmodified parent duplex, AL-DUP-1000, was used to establish the serum stability baseline for evaluating the effect of chemical modifications on nuclease resistance.

AL-DUP-1000

| 5′-CUUACGCUGAGUACUUCGAdTdT-3′ | ALN- | SEQ |
|---|---|---|
| | SEQ- | ID |
| | 1000 | NO: |
| 3′ dTdTGAAUGCGACUCAUGAAGCU-5′ | ALN- | SEQ |
| | SEQ- | ID |
| | 1001 | NO: |

AL-DUP-1000 was subjected to the serum stability assay to evaluate its inherent nuclease resistance and to define its degradation pattern. Denaturing gel electrophoresis was used to analyze AL-DUP-1000 in a human serum stability assay. An siRNA duplex containing 5′ end-labeled sense RNA (*s/as) and a duplex containing 5′ end-labeled antisense RNA (as/s*) were each incubated in 90% human serum and time points were assayed at 10 seconds, 5 minutes, 15 minutes, 30 minutes, 1 hour, 2 hours and 4 hours. The control was a 4 hour time point for siRNA duplex incubated in PBS buffer alone, OH— was the partial alkaline hydrolysis marker. This unmodified duplex was observed to be degraded by both 3′-5′ exonucleases and endonucleases.

Cleavage of the 3′ end of both the sense and antisense strands by 3′-5′ exonucleases occurs within the first 5 minutes of incubation resulting in the loss of the 3′ terminal dT residues. In addition to exonuclease degradation, both strands were cleaved by endonucleases. There was a major endonuclease site at position sixteen of the antisense strand that appears as early as 10 seconds. Very little full length sense or antisense strand was remaining after 1 hour in human serum. Chemical modifications were introduced within the context of the parent duplex to evaluate their effect on nuclease resistance. These chemical modifications fall within one of the following classes: backbone modification, sugar modification, nucleobase modification, cationic modification and conjugate.

Backbone modifications enhanced nuclease resistance: Specific phosphodiester linkages of the siRNA duplex were replaced by either phosphorothioate or methylphosphonate and their stability was evaluated in the human serum stability assay. Table 1 contains the sequences of the duplexes tested. Substitution of the phosphodiester linkage at the 3′ end of both the sense and antisense strands inhibited exonucleolytic degradation of the 3′ overhangs as compared to the unmodified parent duplex. Full length starting material was present for four hours for both the sense and antisense strands. The endonucleolytic cleavage pattern seen in the unmodified duplex was unchanged. Similar results were obtained for duplexes that contained additional phosphorothioates at their 3′ ends. The placement of phosphorothioates at the endonucleolytic cleavage sites (duplexes 1419, 1420 and 1421) did not inhibit endonucleolytic cleavage at these sites. In summary, a single phosphorothioate or methylphosphonate between the two 3′ terminal nucleotides was sufficient to protect the 3′ ends from exonuclease degradation. Additional phosphorothioates at the 3′ ends appear to enhance this effect, which may be necessary for long term exposure to serum nucleases.

TABLE 1
siRNA duplexes containing backbone modifications.

| Alnylam | | Alnylam |
|---|---|---|
| Duplex | Duplex Sequence | Sequence |
| AL-DUP-1393 | 5′-CUUACGCUGAGUACUUCGAdT*dT-3′ | AL-SEQ-1026 |
| | 3′-dT*dTGAAUGCGACUCAUGAAGCU-5′ | AL-SEQ-1027 |
| AL-DUP-1394 | 5′-CUUACGCUGAGUACUUCGA*dT*dT-3′ | AL-SEQ-1028 |
| | 3′-dT*dT*GAAUGCGACUCAUGAAGCU-5′ | AL-SEQ-1029 |
| AL-DUP-1395 | 5′-CUUACGCUGAGUACUUCG*A*dT*dT-3′ | AL-SEQ-1030 |
| | 3′-dT*dT*G*AAUGCGACUCAUGAAGCU-5′ | AL-SEQ-1031 |
| AL-DUP-1396 | 5′-CUUACGCUGAGUACUUC*G*A*dT*dT-3′ | AL-SEQ-1032 |
| | 3′-dT*dT*G*A*AUGCGACUCAUGAAGCU-5′ | AL-SEQ-1033 |
| AL-DUP-1419 | 5′-CUUACGCUGAGU*ACUUCGAdTdT-3′ | AL-SEQ-2182 |
| | 3′-dTdTGAAUGCGACUCA*UGAAGCU-5′ | AL-SEQ-2184 |
| AL-DUP-1420 | 5′-CUU*ACGCUGAGU*ACUUCGAdTdT-3′ | AL-SEQ-2183 |
| | 3′-dTdTGAA*UGCGACUCA*UGAAGCU-5′ | AL-SEQ-2185 |
| AL-DUP-1421 | 5′-CUU*ACGCUGAGU*ACUUCGAdT*dT-3′ | AL-SEQ-2186 |
| | 3′-dT*dTGAA*UGCGACUCA*UGAAGCU-5′ | AL-SEQ-2188 |
| AL-DUP-1329 | 5′-CUUACGCUGAGUACUUCGAdT [symbol] dT-3' | AL-SEQ-1038 |
| | 3′-dT [symbol] dTGAAUGCGACUCAUGAAGCU-5′ | AL-SEQ-1039 |

(* = phosphorothioate, [symbol] = methylphosphonate)

Sugar modifications enhanced nuclease resistance: The effect of replacing the 2′OH with 2′OMe was evaluated at the sites of endonucleolytic cleavage as well as at the 3′ ends of the siRNA duplex. The duplexes tested in the human serum stability assay are shown in Table 2. Some of these duplexes also contained phosphorothioate linkages to evaluate whether the combination of the two modifications enhance nuclease resistance more significantly. Substitution of the terminal dT residues with 2′OMe-U (AL-DUP-1027) reduced 3′-5′ exonuclease degradation slightly over the unmodified parent duplex; however, the extent of exonuclease protection by 2′ OMe-U was far less than that achieved by placing a phosphorothioate between the two terminal dT residues. Addition of a single phosphorothioate between the two terminal 2′ OMe-uridine residues effectively inhibited 3′-5′ exonucleolytic cleavage, as was observed for duplexes AL-DUP-1036, AL-DUP-13ff, and AL-DUP-1363. 2′OMe substitution on its own was much more effective at protecting from endonucleolytic cleavage when placed at the internal cleavage sites. The parent duplex was cleaved 3′ of U at two UpA sites within the duplex. Both strands are cleaved due to the symmetry of this dinucleotide repeat and mapping data was used to confirm the sites of cleavage. Placement of 2′ OMe at the strong endonucleolytic site resulted in inhibition of cleavage at this site. The second, weaker endonucleolytic site, however, was slightly enhanced when the strong site was protected with 2′ OMe. Protection of both sites with 2′OMe (AL-DUP-1363) resulted in reduced endonucleolytic cleavage at both sites. The inhibitory effect of the 2′OMe substitution is consistent with the mechanism of endonucleolytic cleavage, which requires the 2′OH as a nucleophile in the cleavage reaction. 2′ OMe modification will also be an effective means to protect the 3′ overhang of single overhang siRNA duplexes where the 3′ overhang is composed of ribonucleotides. In this situation, 2′OMe substitution can be used to block the possible loss of the terminal two nucleotides by endonucleolytic cleavage and phosphorothioate can be used to protect from exonuclease degradation.

TABLE 2
siRNA duplexes containing 2′OMe substitutions.

| Alnylam | | Alnylam |
|---|---|---|
| Duplex | Duplex Sequence | Sequence |
| AL-DUP-1027 | 5′-CUUACGCUGAGUACUUCGAUU-3′ | AL-SEQ-1006 |
| | 3′-UUGAAUGCGACUCAUGAAGCU-5′ | AL-SEQ-1007 |
| AL-DUP-1036 | 5′-C*UUACGCUGAGUACUUCGAU*U-3′ | AL-SEQ-1008 |
| | 3′-U*UGAAUGCGACUCAUGAAGC*U-5′ | AL-SEQ-1009 |
| AL-DUP-13ff | 5′-C*UUACGCUGAGUACUUCGAU*U-3′ | AL-SEQ-gggg |
| | 3′-U*UGAAUGCGACUCAUGAAGC*U-5′ | AL-SEQ-hhhh |
| AL-DUP-1363 | 5′-C*UUACGCUGAGUACUUCGAU*U-3′ | AL-SEQ-1162 |
| | 3′-U*UGAAUGCGACUCAUGAAGC*U-5′ | AL-SEQ-1163 |

(U = 2′OMe-uridine, * = phosphorothioate)

Cationic modifications enhanced nuclease resistance: The effect of three different cationic chemical modifications on nuclease resistance was tested and compared to the parent unmodified duplex. The structures of the three cationic modifications tested are shown below.

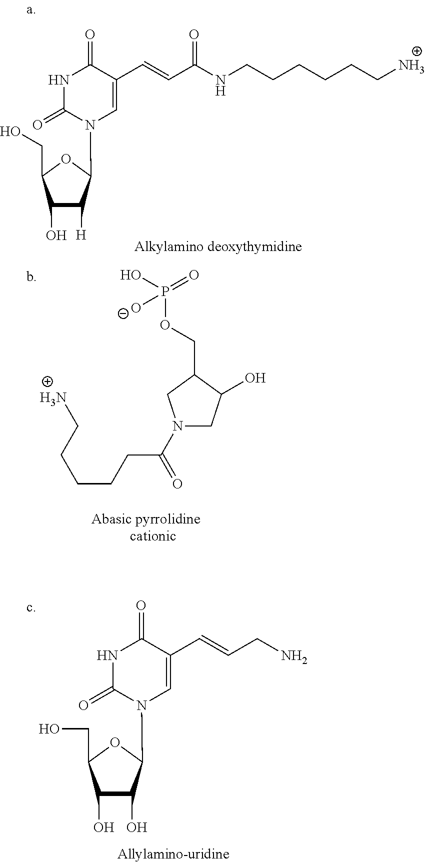

TABLE 3
siRNA duplexes containing cationic substitutions.

| Alnylam | | Alnylam |
|---|---|---|
| Duplex | Duplex Sequence | Sequence |
| AL-DUP-10aa | 5′-CUUACGCUGAGUACUUCGAdTaadT-3′ | AL-SEQ-1017 |
| | 3′-aadTdTGAAUGCGACUCAUGAAGCU-5′ | AL-SEQ-1018 |
| AL-DUP-10bb | 5′-CUUACGCUGAGUACUUCGAaadTaadT-3′ | AL-SEQ-1015 |
| | 3′-aadTaadTGAAUGCGACUCAUGAAGCU-5′ | AL-SEQ-1016 |
| AL-DUP-1ccc | 5′-CUUACGCUGAGUACUUCGAdTdT [symbol] -3′ | AL-SEQ-dddd |
| | 3′- [symbol] dTdTGAAUGCGACUCAUGAAGCU-5′ | AL-SEQ-eeee |
| AL-DUP-1403 | 5′-C*U [symbol] ACGCUGAGUACUUCGAU*U-3′ | AL-SEQ-2080 |
| | 3′-U*UGAA [symbol] GCGACUCAUGAAGC*U-5′ | AL-SEQ-2081 |
| AL-DUP-1406 | 5′-C*U [symbol] ACGCUGAG [symbol] ACUUCGAU*U-3′ | AL-SEQ-2082 |
| | 3′-U*UGAA [symbol] GCGACUCA [symbol] GAAGC*U-5′ | AL-SEQ-2083 |

(aadT = alkylamine-dT, [symbol] = abasic pyrrolidine cationic, [symbol] = allylamino-U, * = phosphorothioate, U = 2′OMe-U)

The sequences of the duplexes assayed in the human serum stability assay are shown in Table 3. Both alkylamino-dT and abasic pyrrolidine cationic modifications were placed at the 3′ terminal overhang to evaluate their effect on 3′-5′ exonuclease degradation. Allylamino-uridines were placed at the internal endonucleolytic cleavage sites to evaluate their ability to inhibit endonucleolytic cleavage. Replacing the 3′ terminal dT residue with a single alkylamino-dT efficiently inhibited 3′-5′ exonucleolytic degradation. Replacement of both dT residues in the overhang with alkylamino-dT resulted in a similar extent of inhibition. Addition of an abasic pyrrolidine cationic modification at the 3′ terminus of each strand also protected against exonucleolytic degradation. Both the alkylamino-dT and abasic pyrrolidine modifications protected from 3′-5′ exonucleolytic cleavage up to 23 hours. Placement of allylamino-U at the internal cleavage sites inhibited endonucleolytic cleavage. The ends of this duplex were stabilized from exonucleolytic degradation by 2′OMe-U and phosphorothioate substitutions in order to separate the two different cleavage events. Endonucleolytic cleavage was inhibited at both internal cleavage sites by allylamino-U substitution for AL-DUP-1406.

3′ conjugates enhanced nuclease resistance: Conjugation of naproxen and ibuprofen to the 3′ end of the siRNA were tested for their ability to inhibit 3′-5′ exonucleolytic degradation. The structure of naproxen is shown in below:

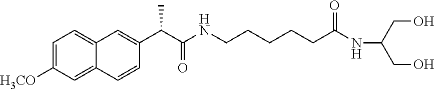

Table 4 lists the siRNAs that were tested in the human serum stability assay. Conjugation of either naproxen or ibuprofen to the 3′ end inhibited exonucleolytic degradation. A naproxen modified duplex (AL-DUP-1069) was as stable in human serum as was the duplex AL-DUP-1413. Presumably the conjugates inhibit exonucleolytic cleavage by sterically blocking the exonuclease from binding to the 3′ end of the siRNA duplex. Similar data was also obtained for AL-DUP-1069 in pooled mouse serum.

TABLE 4
siRNA duplexes containing 3′ conjugates.

| Alnylam | | |
|---|---|---|
| Duplex | Duplex Sequence | SEQ ID NO: |
| AL-DUP-1069 | 5′-CUUACGCUGAGUACUUCGAdTdTNap-3′ | |
| | 3′-NapdTdTGAAUGCGACUCAUGAAGCU-5′ | |
| AL-DUP-1413 | 5′-CUUACGCUGAGUACUUCGAdTdTIbu-3′ | |
| | 3′-NapdTdTGAAUGCGACUCAUGAAGCU-5′ | |

(Nap = Naproxen, Ibu = Ibuprofen)

### Example 2

### siRNA Modifications Reduce Immune Stimulatory Effect of siRNAs

Isolation of Human PBMCs: A concentrated fraction of leukocytes (buffy coat) was obtained from the Blood Bank Suhl, Germany. These cells were negative for a variety of pathogens, including HIV, HCV, and others. The buffy coat was diluted 1:1 with PBS, added to a tube containing Ficoll, and centrifuged for 20 minutes at 2200 rpm to allow fractionation. This was followed by removal of the turbid layer of white blood cells and transference to a tube with fresh PBS and Ficoll, which was centrifuged for 15 minutes at 2200 rpm. The turbid layer of white blood cells was again removed, transferred to RPMI 1640 culture medium and centrifuged for 5 minutes at 1,200 rpm to pellet the white blood cells. The cells were resuspended in RPMI 1640, pelleted as above, and resuspended in media with 10% FCS to 1×10⁶/mL

Interferon-alpha Measurement: Cells in culture were combined with 500 nM oligonucleotide in Optimem or 133 nM oligonucleotide with GP2 or Lipofectamine-2000 (Invitrogen, Carlsbad, Calif.) transfection agent for 24 hours at 37° C. Interferon-alpha was measured with Bender MedSystems' (Vienna, Austria) instant ELISA kit according to manufacturer's instructions, except that 50 μl instead of 20 μl of culture supernatant was used.

iRNA agents used

| Duplex | | |
|---|---|---|
| | ApoB siRNAs, unconjugated | |
| AL-DP-5386 | GAuuGAuuGACCuGUCcAuUsC | pGAAuGGAcAGGUcAAUcAAUcsusU |
| AL-DP-5431 | GAUUGAUUGACCUGUCCAUUsC | pGAAUGGACAGGUCAAUCAAUcsusU |
| AL-DP-5390 | cACcAACuUCuUCcACGAGUsC | pGACUCGuGGAAGAAGuuGGugsusU |
| AL-DP-5435 | CACCAACUUCUUCCACGAGUsC | pGACUCGUGGAAGAAGUUGGUgsusU |
| AL-DP-5395 | GAGuuuGuGAcAAAuAuGGGsC | pGCCCAuAuuuGUcAcAAACUcscsA |
| AL-DP-5440 | GAGUUUGUGACAAAUAUGGGsC | pGCCCAUAUUUGUCACAAACUcscsA |
| AL-DP-5398 | CuuuAcAAGCCuuGGuUcAGsU | pACuGAACcAAGGCUuGuAAAgsusG |
| AL-DP-5443 | CUUUACAAGCCUUGGUUCAGsU | pACUGAACCAAGGCUUGUAAAgsusG |
| AL-DP-5399 | GGAAUCuuAuAuuuGAUCcAsA | puuGGAUcAAAuAuAAGAuUCcscsU |
| AL-DP-5444 | GGAAUCUUAUAUUUGAUCCAsA | pUUGGAUCAAAUAUAAGAUUCcscsU |
| | ApoB siRNAs with 3′ chol | |
| AL-DP-5536 | GAuuGAuuGACCuGUCcAuUCsChol | GAAuGGAcAGGUcAAUcAAUcsusU |
| AL-DP-5537 | GAUUGAUUGACCUGUCCAUUCsChol | GAAUGGACAGGUCAAUCAAUcsusU |
| AL-DP-5538 | cACcAACuUCuUCcACGAGUCsChol | GACUCGuGGAAGAAGuuGGugsusU |
| AL-DP-5539 | CACCAACUUCUUCCACGAGUCsChol | GACUCGUGGAAGAAGUUGGUgsusU |
| AL-DP-5540 | GAGuuuGuGAcAAAuAuGGGCsChol | GCCCAuAuuuGUcAcAAACUcscsA |
| AL-DP-5541 | GAGUUUGUGACAAAUAUGGGCsChol | GCCCAUAUUUGUCACAAACUcscsA |
| AL-DP-5542 | CuuuAcAAGCCuuGGuUcAGUsChol | ACuGAACcAAGGCUuGuAAAgsusG |
| AL-DP-5543 | CUUUACAAGCCUUGGUUCAGUsChol | ACUGAACCAAGGCUUGUAAAgsusG |
| AL-DP-5544 | GGAAUCuuAuAuuuGAUCcAAsChol | uuGGAUcAAAuAuAAGAuUCcscsU |
| AL-DP-5545 | GGAAUCUUAUAUUUGAUCCAAsChol | UUGGAUCAAAUAUAAGAUUCcscsU |
| AL-DP-5546 | GUCAUCACACUGAAUACCAAUChol | AUUGGUAUUCAGUGUGAUGACAC |

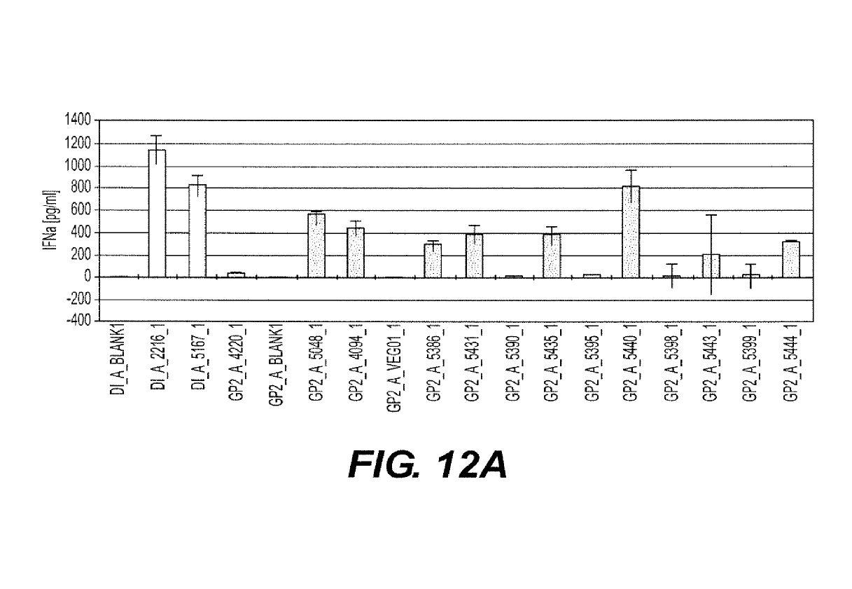

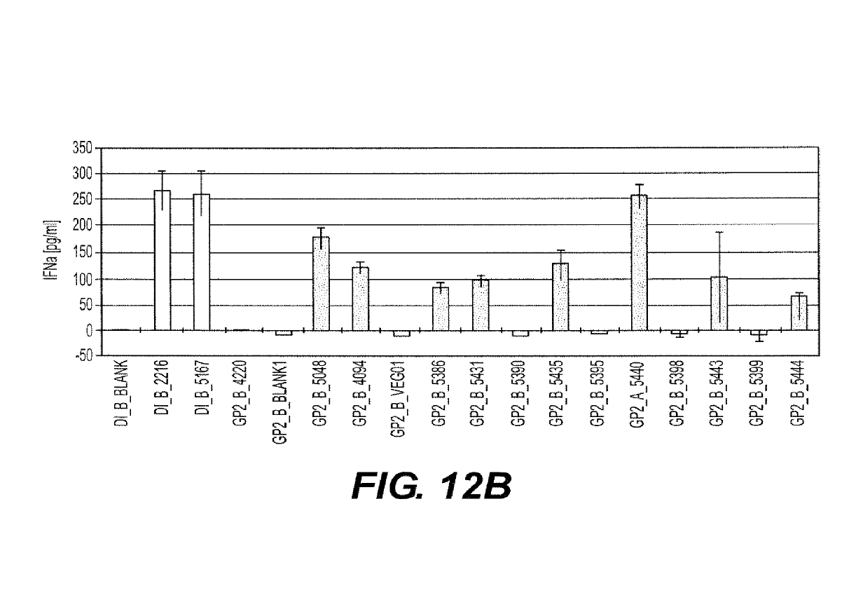

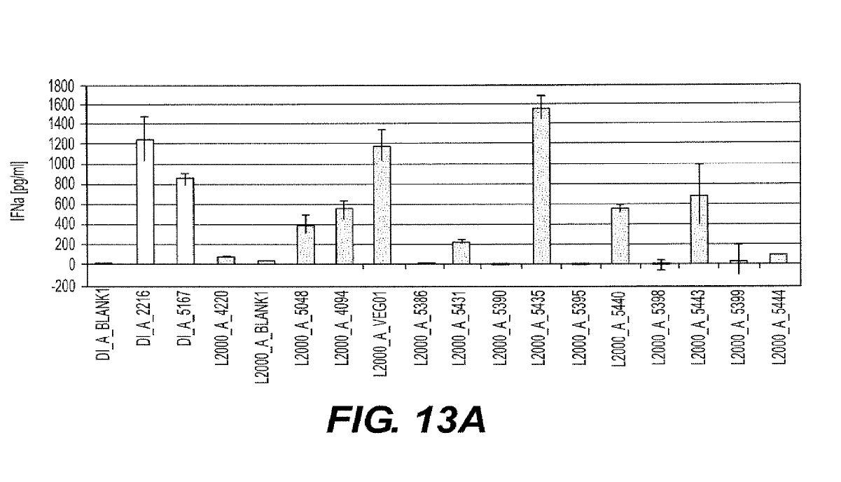

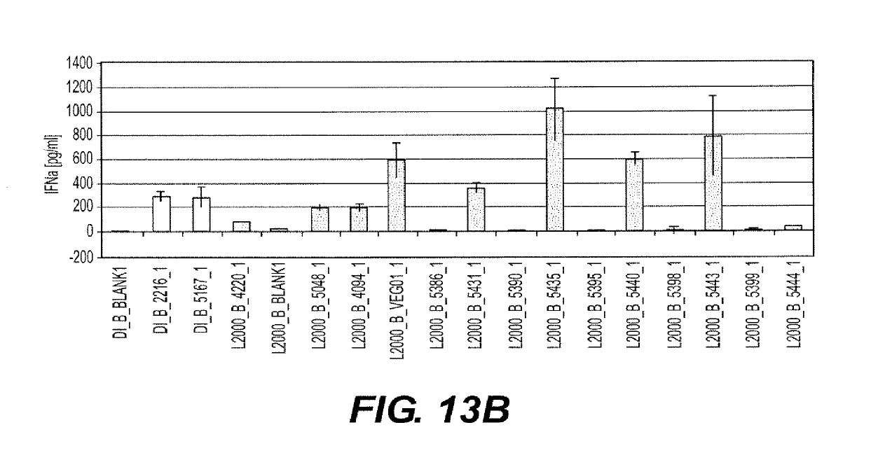

Results and Conclusions. FIGS. 12A and 12B, and 13A and 13B provide the results of IFN-alpha induction assays using the iRNA agents provided in the table above. As can be seen, extensive internal 2′-O methyl modification can be used to reduce the immunostimulatory effect of an iRNA agent.

A number of embodiments of the invention have been described. Nevertheless, it will be understood that various modifications may be made without departing from the spirit and scope of the invention. Other embodiments are in the claims.

What is claimed is:

1. A method of evaluating an iRNA agent comprising:
(a) providing a sense strand and an antisense strand sufficiently complementary to hybridize to the sense strand, wherein the sense and/or antisense strand comprises at least one ribonucleotide modification;
(b) annealing the first and second oligonucleotides to produce a candidate iRNA agent;
(c) contacting the candidate iRNA agent with a cell-free system, cell, or animal; and
(d) evaluating the immune response in the cell-free system, cell, or animal as compared to an immune response in a cell-free system, cell, or animal contacted with an iRNA agent comprising an unmodified sense and antisense strands
wherein a candidate iRNA agent that stimulates an immune response to a lesser or greater extent than a reference iRNA agent comprising unmodified sense and antisense strands is determined to be an iRNA agent that modulates an immune system response.

2. The method of claim 1, wherein the antisense strand is 30 or fewer nucleotides in length, and the duplex region of the candidate iRNA agent is 15-30 nucleotide pairs in length.

3. The method of claim 1, wherein the evaluating step comprises assaying for an increase or decrease in expression of a cytokine or interleukin protein.

4. The method of claim 3, wherein the evaluating step comprises assaying for an increase or decrease in expression of an interleukin protein, and wherein the interleukin protein is IL-10, IL-12, or IL-6.

5. The method of claim 1, the evaluation step further comprises testing the iRNA agents to determine the interaction with a protein of the immune system.

6. The method of claim 5, wherein the protein of the immune system is a cell-surface receptor, growth factor, cytokine, or interleukin protein.

7. The method of claim 6, wherein the cell surface receptor is a toll-like receptor.

8. The method of claim 1, wherein the antisense strand is sufficiently complementary to hybridize to a target nucleic acid sequence in a cell, and the method further comprises performing an assay to determine whether the iRNA agent decreases the amount of target nucleic acid in the cell.

9. The method of claim 8, wherein the iRNA agent decreases the amount of target nucleic acid in the cell, and the iRNA agent interacts with a protein component of the immune system, wherein the protein component is selected from the group consisting of a cytokine, a cytokine receptor, and a Toll-like receptor.

10. The method of claim 1, wherein the at least one ribonucleotide modification is a substitution of an adenine with a 2-substituted purine, a 6-substituted purine, a 7-deaza-alkyl-substituted purine, a 7-deaza-alkenyl-substituted purine, a 7-deaza-alkynyl-substituted purine, or a purine that is not adenine.

11. The method of claim 10, wherein the 2-substituted purine is a 2-amino-adenine.

12. The method of claim 1, wherein the at least one ribonucleotide modification is a substitution of a guanine with an inosine, an aminopurine, 2-substituted guanine, 7-deaza-alkyl-substituted guanine, 7-deaza-alkenyl-substituted guanine, 7-deaza-alkynyl-substituted guanine, or an O-6 alkylated guanine.

13. The method of claim 1, wherein the at least one ribonucleotide modification is a substitution of a cytosine with a 5-substituted cytosine, an N-4 substituted cytosine, a G-clamp, an analog of a G-clamp, a 2-thio-cytosine, 4-thio-cytosine, or a uracil.

14. The method of claim 12, wherein the 5-substituted cytosine is a 5-methyl cytosine.

15. The method of claim 1, wherein the at least one ribonucleotide modification is a substitution of a uracil with a 5-substituted uracil, 4-thio-uracil, 2-thio-uracil, 5-methyl-2-thio-uracil, pseudouridine, 1-alkylpseudouridine, or 3-alkylpseudouridine.

16. The method of claim 1, wherein the at least one ribonucleotide modification is a 2′-fluoro, 2′-O-methyl, 2′-O-alkyl, 2′-O-alkoxyalkyl, 2′-O-methoxyethyl, 2′-O-allyl, 2′-O-propyl, 2′-O—(N-methyl-acetamide (NMA), or 2′-O—(N,N-dimethylaminooxyethyl) modification.

17. The method of claim 1, wherein the at least one ribonucleotide modification comprises a ribonucleoside that has been replaced with an arabinose-containing nucleoside, a 2′-fluoroarabinose-containing nucleoside, or a 2′-O-methylarabinose-containing nucleoside.

18. The method of claim 1, wherein the at least one ribonucleotide modification comprises a ribonucleoside that has been replaced with a 2′-deoxynucleoside, a 2′-fluorodeoxynucleoside, or a 2′-O-methyldeoxynucleoside.

19. The method of claim 1, wherein the sense and/or antisense strand further comprises at least one backbone modification selected from the group consisting of a phosphorothioate, a boronaphosphate, a methylphosphonate, and a dithioate modification.

20. The method of claim 19, wherein the sense and/or antisense strand is substantially free of stereogenic phosphorus atoms having an R^{p} configuration.

21. The method of claim 19, wherein the sense and/or antisense strand is substantially free of stereogenic phosphorus atoms having an Sp configuration.

22. The method of claim 1, wherein the sense and/or antisense strand comprises at least two sugar modifications, and wherein the at least two sugar modifications comprise 2′-modifications of the ribose substituents of each ribonucleotide.

23. The method of claim 22, wherein the 2′-modifications of the ribose substituents are each independently selected from the group consisting of a 2′-fluoro, 2′-O-methyl, 2′-O-alkyl, 2′-O-alkoxyalkyl, 2′-O-methoxyethyl, 2′-O-allyl, 2′-O-propyl, 2′-O—(N-methyl-acetamide (NMA), and 2′-O—(N,N-dimethylaminooxyethyl) modification.

24. The method of claim 1, wherein the iRNA agent comprises at least one ribonucleotide modification on the sense strand and at least one ribonucleotide modification on the antisense strand, wherein the at least one ribonucleotide modification on the antisense strand differs from the at least one ribonucleotide modification on the sense strand.

25. A method of treating a human comprising administering an iRNA agent evaluated by the method of claim 1.

26. The method of claim 25, further comprising observing the human to determine whether the human has an immunostimulatory response following administration of the iRNA agent.

27. The method of claim 26, comprising measuring the level of a protein component of the immune system present in a biological sample from the human, wherein if the level of the protein component is higher after administering the iRNA agent than before administering the iRNA agent, then the human is determined to have an immunostimulatory response.

28. The method of claim 27, wherein the protein component is a cytokine, e.g., an interleukin or interferon protein.

29. The method of claim 28, wherein the interleukin protein is IL-10, IL-12, or IL-6.

30. The method of claim 27, wherein the protein component is a Toll-Like receptor selected from the group consisting of TLR3, TLR7, TLR8, and TLR9.

31. The method of claim 28, wherein the cytokine is expressed in T cells, B cells, monocytes, macrophages, dendritic cells, or natural killer cells of the human.

32. The method of claim 25, wherein the human is immunocompromised and the iRNA agent stimulates an immune response in a cell to a greater extent than an iRNA agent comprising unmodified sense and antisense strands.

33. A method of increasing the ratio of double stranded iRNA (dsiRNA) agent to single stranded iRNA (ssiRNA) agent in a human, wherein the dsiRNA agent is administered to the human, and wherein the dsiRNA agent comprises one or more modifications that inhibit disassociation of the dsiRNA agent as compared to a dsiRNA agent that does not comprise the one or more modifications.

34. The method of claim 33, wherein the one or more modifications comprise a chemical linker selected from the group consisting of a hexaethylene glycol linker, poly(oxyphosphinico-oxy-1,3-propandiol) linker, allyl linker, and polyethylene glycol linker.

35. The method of claim 33, wherein the one or more modifications increases the melting temperature of the dsiRNA agent as compared to a dsiRNA agent that does not comprise the one or more modifications.

36. The method of claim 33, wherein the one or more modifications comprises a locked nucleic acid, G-clamp, 2′-O-methyl, 2′-fluoro, 2′-O-methoxyethyl, 2-thio-pyrimidine, 2-amino-adenine or pseudouridine.

37. The method of claim 33, wherein the one or more modifications of the dsiRNA agent occur only in the sense strand of the dsiRNA agent.

38. The method of claim 33, wherein the one or more modifications of the dsiRNA agent occur only in the antisense strand of the dsiRNA agent.

39. A method of evaluating an iRNA agent comprising:
(a) providing a candidate single stranded iRNA agent that comprises at least one ribonucleotide modification;
(b) contacting the candidate single stranded iRNA agent to a cell-free system, cell, or animal; and
(c) evaluating the immune response in the cell-free system, cell, or animal as compared to an immune response in a cell-free system, cell, or animal contacted with an unmodified single stranded iRNA agent,
wherein a candidate single stranded iRNA agent that stimulates an immune response to a lesser or greater extent than a reference unmodified iRNA agent is determined to be an iRNA agent that modulates an immune system response.

40. The method of claim 39, wherein the candidate single-stranded iRNA agent is 15-2000 nucleotides in length.
